(19) United States
(12) Patent Application Publication
(10) Pub. No.: US 2005/0033127 A1
(43) Pub. Date: Feb. 10, 2005

(54) Wireless blood glucose monitoring system

(75) Inventors: Emil W. Ciurczak, Goldens Bridge, NY (US); Gary Ritchie, Kent, CT (US); Howard Mark, Suffern, NY (US); Kevin C. Bynum, Yonkers, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Euro-Celtique, S.A., Luxembourg (LU)

(21) Appl. No.: 10/766,640

(22) Filed: Jan. 27, 2004

Related U.S. Application Data
(60) Provisional application No. 60/443,770, filed on Jan. 30, 2003.

(51) Int. Cl. A61B 5/00

(52) U.S. Cl. 600/316; 600/322

(57) ABSTRACT

A system for predicting blood glucose values in a patient includes a remote wireless non-invasive spectral device, the remote wireless non-invasive spectral device generating a spectral scan of a body part of the patient. Also included are a remote invasive device and a central processing device. The remote invasive device generates a constituent value for the patient, while the central processing device predicts a blood glucose value for the patient based upon the spectral scan and the constituent value.

### CROSS-REFERENCE TO RELATED APPLICATIONS

This application is related to U.S. Application Serial PCT/US 01/25165, filed Aug. 10, 2001, and U.S. application Ser. No. 09/636,041, filed Aug. 10, 2000, the entire disclosures of which are hereby incorporated by reference. The application is also related to U.S. application Ser. No. 09/932,185.

### BACKGROUND

The present invention relates to the field of near infrared spectrometry for predicting patient blood glucose levels.

There are roughly four million people in the United States currently diagnosed with Diabetes Mellitus. This disease causes blindness, loss of extremities, heart failure, and other complications over time. There is no “cure” for the disease, which is caused by either the failure of the pancreas to produce insulin or the body's inability to use insulin, but rather only treatment, most commonly with insulin injections in order to change the blood-glucose level.

The majority of patients with Type I diabetes, as well as people with Type II diabetes or diagnosed as pre-diabetic, need to frequently monitor their blood glucose levels, establishing an individual blood glucose profile in order to adjust diet, medication, exercise, or to lower the blood glucose while avoiding hypoglycemia (low blood-sugar). In well-regulated patients, two or three blood samples are tested for glucose daily and are usually sufficient. In new or difficult patients, or when monitoring for hypoglycemia is required, blood samples may be required in rapid (every few minutes) succession.

Tests for blood glucose levels consist of obtaining blood by venipuncture or pricking an extremity (usually a finger) to draw a drop of blood. This blood sample is inserted into an analytical device (e.g., Accu-Check®, Hemo-Cue®, Diasensor 1000®, Coming Express 550®, Roche Cobas Mira®, or Ektachem (R)DT60II Analyzer®). The device may be an electrochemical detector that monitors reaction of glucose oxidase with glucose in the blood. The current or voltage is measured, and resulting data is displayed as a concentration, typically milligrams per deciliter (mg/dL). Another means of measuring glucose is by measuring the absorbence of the reaction at 540 nm. It is often difficult, particularly in elderly or infant patients, to perform the necessary measurement, particularly when needed several times a day.

As a result, a need has developed for non-invasive techniques useable in predicting the concentration of blood glucose in the bloodstream of a patient. In this regard, a significant number of researchers have attempted over the past few decades to develop non-invasive glucose monitors using near-infrared (NIR) spectrometers.

Infrared (IR) spectrometry is a technique that is based upon the vibrational changes of the atoms of a molecule. In accordance with infrared spectrometry, an infrared spectrum is generated by transmitting infrared radiation through a sample of an organic substance and determining the portions of the incident radiation that are absorbed by the sample. An infrared spectrum is a plot of absorbence (or transmittance) against wavenumber, wavelength or frequency. Infrared radiation (IR) may be roughly divided into three wavelength bands: near-infrared radiation, mid-infrared radiation, and far-infrared radiation. Near-infrared radiation (NIR) is radiation having a wavelength between about 750 nm and about 3000 nm. Mid-infrared radiation (MIR) is radiation having a wavelength between about 3000 and about 10,000 nm. Far-infrared radiation (FIR) is radiation having a wavelength between about 10,000 nm and about 1000 μm (1000 μm being the beginning of the microwave region). The desired range may be chosen to suit the analysis being performed.

A variety of different types of spectrometers are known in the art such as grating spectrometers, FT (Fourier transformation) spectrometers, Hadamard transformation spectrometers, AOTF (Acousto Optical Tunable Filter) spectrometers, diode array spectrometers, filter-type spectrometers, ATR (attenuated total reflectance), scanning dispersive spectrometers and nondispersive spectrometers.

Filter-type spectrometers, for example, utilize an inert solid heated to provide continuous radiation (e.g., tungsten filament lamp) to illuminate a rotating opaque disk, wherein the disk includes a number of narrow bandpass optical filters. The disk is then rotated so that each of the narrow bandpass filters passes between the light source and the sample. An encoder indicates which optical filter is presently under the light source. The filters filter light from the light source so that only a narrow selected wavelength range passes through the filter to the sample. Optical detectors are positioned so to as detect light that either is reflected by the sample (to obtain a reflectance spectra) or is transmitted through the sample (to generate a transmittance spectra). The amount of detected light is then measured and provides an indication of the amount of absorbence of the light by the substance under analysis.

Linear variable filter spectrometers include a linear variable filter which may be used to filter light from a light source so that a sample under analysis is irradiated with at least one specified band of wavelengths, the specified band being variable. Alternatively, a linear variable filter may be positioned upstream of a detector so that only a specified, variable band of wavelengths of light reaches the detector.

Diode Array spectrometers use infrared emitting diodes (IREDs) as sources of near-infrared infrared radiation. A plurality of (for example, eight) IREDs are arranged over a sample work surface to be illuminated for quantitative analysis. Near-infrared radiation emitted from each IRED impinges upon an accompanying optical filter. Each optical filter is a narrow bandpass filter that passes NIR radiation at a different wavelength. NIR radiation passing through the sample is detected by a detector (such as a silicon photodetector). The amount of detected light is then measured and provides an indication of the amount of absorbence of the light by the substance under analysis.

Acousto Optical Tunable Filter spectrometers utilize an RF signal to generate acoustic waves in a TeO₂ crystal. A light source transmits a beam of light through the crystal, and the interaction between the crystal and the RF signal splits the beam of light into three beams: a center beam of unaltered white light and two beams of monochromatic and orthogonally polarized light. A sample is placed in the path of one of the monochromatic beam detectors, which are positioned to detect light that either is reflected by the sample (to obtain a reflectance spectra) or is transmitted through the sample (to generate a transmittance spectra). The wavelength of the light source is incremented across a wavelength band of interest by varying the RF frequency. The amount of detected light is then measured and provides an indication of the amount of absorbance of the light by the substance under analysis.

In grating monochromator spectrometers, a light source transmits a beam of light through an entrance slit and onto a diffraction grating (the dispersive element) to disperse the light beam into a plurality of beams of different wavelengths (i.e., a dispersed spectrum). The dispersed light is then reflected back through an exit slit onto a detector. By selectively altering the path of the dispersed spectrum relative to the exit slit, the wavelength of the light directed to the detector can be varied. The amount of detected light is then measured and provides an indication of the amount of absorbance of the light by the substance under analysis. The width of the entrance and exit slits can be varied to compensate for any variation of the source energy with wavenumber.

In an ATR spectrometer, radiant energy incident on an internal surface of a high refractive index transparent material is totally reflected. When an infrared absorbing material is in optical contact with the totally internally reflecting surface, the intensity of the internally reflected radiation is diminished for those wavelengths or energies where the material absorbs energy. Since an internal reflecting surface is essentially a perfect mirror, the attenuation of this reflected intensity by a material on its surface provides a means of producing an absorption spectrum of the material. Such spectra are called internal reflection spectra or attenuated total reflection (ATR) spectra. An ATR spectrometer, as described herein, refers to any type of spectrometer (e.g., grating, FT, AOTF, filter) which includes, as a component part, an ATR crystal.

The material with the high index of refraction that is used to create internal reflection is called an internal reflection element (IRE) or an ATR crystal. The attenuation of the internally reflected radiation results from the penetration of the electromagnetic radiation field into the matter in contact with the reflection surface. This field was described by N. J. Harrick (1965) as an evanescent wave. It is the interaction of this field with the matter in contact with the IRE interface that results in attenuation of the internal reflection.

A nondispersive infrared filter photometer is designed for quantitative analysis of various organic substances. The wavelength selector comprises: a filter as previously described to control wavelength selection; a source; and a detector. The instrument is programmed to determine the absorbance of a multicomponent sample at wavelengths and then to compute the concentration of each component.

As stated above, spectrometers have been used to measure the chemical composition of blood and, more particularly, blood glucose. The mean blood glucose level varies within a normal range of 70-120 mg/dL from person to person. For diabetics, these fluctuations can vary markedly, reaching values of 200-400 mg/dL within a very short time in accordance with their food intake, physical activity, a complication by another disease, or the like. The majority of patients with Type I diabetes, as well as people with Type II diabetes or diagnosed as pre-diabetic, need to frequently monitor their blood glucose levels, establishing an individual blood glucose profile in order to adjust diet, medication or exercise, or in order to lower the blood glucose while avoiding hypoglycemia (low blood-sugar). In well-regulated patients, two or three blood samples are tested for glucose daily and are usually sufficient. In new or difficult patients, or when monitoring for hypoglycemia is required, blood samples may be required in rapid (every few minutes) succession.

The major problems with non-invasive NIR blood glucose monitors are the high operating cost, a lack of reproducible results and difficulty in use. Hand-held instruments for home use fail in that the instruments do not consistently provide the correct assessment of blood glucose concentration over the entire length of time the instruments are used. These hand-held devices are calibrated with a one-time global modeling equation hard-wired into the instrument, to be used by all patients from time of purchase onward. The model does not provide for variations in the unique patient profile which includes such factors as gender, age or other existing disease states.

For example, U.S. Pat. No. 5,961,449 to Toida et al. purports to disclose a method and apparatus for non-invasive measurement of the concentration of glucose in the aqueous humor in the anterior aqueous chamber of the eyeball, and a method and apparatus for non-invasive measurement of the concentration of glucose in the blood in accordance with the concentration of glucose in the aqueous humor. Known near-infrared analytical techniques using multivarient analysis are utilized therein.

A number of patents including U.S. Pat. Nos. 5,703,364, 5,028,787, 5,077,476 and 5,068,536, all to Rosenthal, purport to describe an at-home testing near-infrared quantitative analysis instrument and method of non-invasive measurement of blood glucose by measuring near-infrared energy following interaction with venous or arterial blood or following transmission through a blood-containing body part. Questions have been raised about the accuracy of the instrument described in these patents and, to date, FDA approval for such an instrument has not been attained.

U.S. Pat. No. 5,574,283 to Quintana purports to describe a near-infrared quantitative analysis instrument for measuring glucose comprising an analysis instrument having a removable insert that facilitates positioning of an individual user's finger within the instrument according to the size of the user's finger.

U.S. Pat. No. 5,910,109 to Peters et al. allegedly describes a glucose measuring device for determining the concentration of intravascular glucose in a subject including: a light source having a wavelength of 650, 880, 940 or 1300 nm to illuminate the fluid; receptors associated with the light sources for receiving light and generating a transmission signal representing the light transmitted and adapted to engage a body part of a subject; and a signal analyzer, which includes a trained neural network for determining the glucose concentration in the blood of the subject. This reference purportedly also provides a method for determining the glucose concentration, which method includes calibration of a measuring device and setting of an operating current for illuminating the light sources during operation of the device. According to this patent, when a transmission signal is generated by receptors, the high and low values from each of the signals are stored in the device and are averaged to obtain a single transmission value for each of the light sources. The averaged values are then analyzed to determine the glucose concentration, which then is displayed.

U.S. Pat. No. 5,935,062 to Messerschmidt et al. purports to describe a specular control device that can discriminate between diffusely reflected light that is reflected from selected depths or layers within the tissue by receiving the diffusely reflected light that is reflected from a first layer or depth within the tissue, while preventing the remaining diffusely reflected light from reaching the spectroscopic analyzer. This patent allegedly describes a method for obtaining diffuse reflectance spectra from human tissue for the non-invasive measurement of blood analytes, such as blood glucose by collecting the infrared energy that is reflected from a first depth and rejecting the infrared energy that is reflected from a second.

U.S. Pat. No. 5,941,821 to Chou allegedly provides an apparatus for more accurate measurement of the concentration of a component in blood (e.g., glucose), including a source for irradiating a portion of the blood by heat-diffusion to generate acoustic energy propagating in a second medium over a surface of the blood in response to the irradiation, a detector for detecting the acoustic energy and for providing an acoustic signal in response to the acoustic energy, and a processor for determining the concentration of the component in response to the acoustic signal and characteristics of the component.

In all spectroscopic techniques, including those discussed above, calibration samples must be run before an analysis is conducted. In NIR spectroscopy, a modeling equation (often referred to as a calibration model) that reflects the individual patient's blood glucose profile is generated by scanning a number of blood glucose samples to generate a set of calibration data, and then processing the data to obtain the modeling equation.

In a static system with little interference, this calibration is required only once, and spectral prediction can be conducted without the need to rerun calibration samples. In the real world, this is an infrequent occurrence. Most systems that require study are dynamic and require frequent recalibration. The recalibration procedure involves scanning a set of calibration samples and analyzing those same samples with a primary technique, such as High Performance Liquid Chromatography (HPLC), to adjust the modeling equation.

In previous attempts to develop a near IR spectral device for blood glucose determination, a single static modeling equation was generated using a statistical population of test subjects. This single modeling equation was then “hardwired” into the spectral sensing device and used for all test subjects. This has proven to be problematic since people display blood chemistry within a wide range of normal values, or abnormal values in the case of a disease state (e.g., diabetes), due to each person's combinations of water level, fat level and protein level, each of which cause variations in energy absorption. The variations in these constituents between different people make a universal calibration for diabetes patients unlikely, particularly because the amount of glucose in the body is less than one thousandth of these other constituents.

U.S. Pat. No. 5,507,288 to Bocker et al. purportedly describes a non-invasive portable sensor unit combined with an invasive analytical system that can contain an evaluation instrument capable of calibrating the results of the non-invasive system. The evaluation instrument of this patent contains only one calibration equation, and the disclosure does not contemplate recalculation of the equation or recalibration of the evaluation instrument over time. This can be problematic, since, because a patient's blood chemistry changes with time, the “permanent” calibration slowly, or even rapidly, begins giving incorrect predictions. Thus, the ability to correctly assess the amount of blood glucose deteriorates over time.

Moreover, previous devices use infrared spectrometers that transmit their data relating to spectral scans by a physical connection, rather than by a wireless one. Thus, such spectrometers remain physically connected to devices that interpret the data. The necessity of such a physical connection increases the number of devices necessary to analyze the spectral data and increases the complexity and size of these devices. This is undesirable especially if a remote, possibly hand-held, spectral device for home use is desired.

Throughout this application, various patents and publications are referred to. Disclosure of these publications and patents in their entirety are hereby incorporated by reference into this application to more fully describe the state of the art to which this invention pertains.

### SUMMARY OF THE INVENTION

In order to accurately predict blood glucose levels using a noninvasive spectroscopic technique, a dynamic modeling equation is needed. A dynamic modeling equation is one that provides a way to recalculate the equation when the model no longer accurately reflects the patient's glucose profile. A dynamic model is accomplished by scanning the subject with a noninvasive spectroscopic blood glucose monitor and then using an invasive technique (e.g., venipuncture or a fingerstick) to obtain a constituent value to associate with the spectral data. This procedure must be repeated a number of times in order to obtain a sufficient number of spectral data scans and associated constituent values to develop a robust and accurate modeling equation for the individual patient. The frequency and amount of recalibration needed is dependent on the amount of variation in the individual subject's blood glucose values. To recalibrate, additional spectral scans and associated constituent values are obtained from the patient, and the modeling equation is regenerated using the original data along with the new data. In cases where the original data is found to be unsuitable (for example, due to significant change in the patient's condition), it may be necessary to discard the original data and obtain a full set of new spectral scans and associated constituent values. However, even if this recalibration is required on a weekly basis, a significant reduction in the amount of invasive monitoring has been achieved.

A truly dynamic modeling equation would seemingly require a highly trained and experienced individual using an advanced statistics computer program to evaluate the modeling equation and to perform the mathematics required to maintain the modeling equation. It is impractical, however, to have a scientist directly consult with each patient to maintain his or her individual modeling equation. This is especially true if development of a hand-held, remote spectral device for home use is desired.

Although many attempts have been made to construct a spectral device that will monitor the amount of blood glucose in a non-invasive manner, some of which are discussed above, a major shortcoming in each of these previous attempts has been in the development of a robust dynamic modeling equation for the prediction of blood glucose levels.

In accordance with the present invention, a dynamic modeling equation is provided that can predict the patient's blood glucose level using a noninvasive spectral scan obtained from a remote spectral device (preferably handheld). A spectral scan is obtained from a patient and sent to a central computer. A central computer stores the generated spectral scan along with a previously generated patient modeling equation for that patient. A resultant blood glucose level is calculated for that patient based on his or her individual modeling equation. If the spectral scan falls within the range of the modeling equation, a blood glucose value is predicted and the predicted blood glucose level is output to the patient. If the spectral scan falls outside the range of the modeling equation, regeneration of the model is required, and the patient is instructed to take a number of noninvasive scans, followed by an invasive blood glucose level determination. All of the data is then transferred to the central computer where the modeling equation is regenerated based on both the existing data points and the new data points. A preferred method for generating and updating the modeling equation is set forth in more detail below.

Preferably, the central computer uses a complex statistics computer program to generate a new modeling equation, thereby allowing for much of this task to be automated. A new modeling equation is generated as needed, for example in cases of a change in medical condition that affects the blood glucose levels or as instructed by the manufacturer (e.g., once a month).

The remote spectral device communicates with the central computer by any conventional mode of data transmission, such as a cellular data link, a telephone modem, a direct satellite link, or an Internet link. The remote spectral device may be directly linked to the invasive blood glucose monitor by an appropriate data connection, such as an RS233 data connection, but preferably both the sensor and monitor are contained in the same unit along with a handheld computer, similar to a PALM PILOT™. In certain embodiments, additional messages can be sent from the central computer to the remote spectral device, for example, reminders to the patient to obtain blood glucose levels or to take medication. It may also be desirable to include other data inputs from the patient, such as blood pressure, heart rate and temperature, which data will be transmittable from the remote spectral device to the central computer.

In further embodiments, the central processing unit further communicates the relevant information received from the patient and any instructions transmitted to the patient via the remote spectral device to the patient's doctor or hospital.

In one embodiment of the present invention, there is provided a method for predicting blood glucose values in a patient by: generating an individualized modeling equation for a patient as a function of non-invasive spectral scans of a body part of the patient and an analysis of blood samples from the patient, and storing the individualized modeling equation on a central computer; receiving from the patient a non-invasive spectral scan generated by a remote spectral device; predicting a blood glucose value for the patient as a function of the non-invasive spectral scan and the individualized modeling equation, and transmitting the predicted blood glucose value to the patient; determining that a regeneration of the individualized modeling equation is required, and transmitting a request for a set of non invasive spectral scans and a corresponding set of blood glucose values to the patient; acquiring a set of noninvasive spectral scans from the patient using the remote spectral device and a corresponding set of blood glucose values from a remote invasive blood glucose monitor; transmitting the set of spectral scans and corresponding blood glucose values to the central computer; and regenerating the individualized modeling equation as a function of the set of spectral scans and corresponding blood glucose values.

None of the prior devices provide a system for non-invasively and wirelessly predicting blood glucose values in a patient, such as would be suitable for home use, in a hand-held or table-top manner, for example.

Accordingly, one embodiment the present invention also provides a system for predicting blood glucose values in a patient using a remote wireless non-invasive spectral device which generates a spectral scan of a body part of the patient. The system also includes a remote invasive device for generating a constituent value for the patient is provided. A central processing device, such as a central computer, is also included in the system. The central processing device predicts a blood glucose value for the patient based upon the spectral scan and the constituent value, using the dynamic modeling equation as described above, for example.

The remote wireless non-invasive spectral device may include a wireless spectrometer, which may be an infrared spectrometer. The infrared spectrometer may be a grating spectrometer, a diode array spectrometer, a filter-type spectrometer, an Acousto Optical Tunable Filter spectrometer, a scanning spectrometer, an ATR spectrometer and a nondispersive spectrometer. The wireless spectrometer may include a light source; a focusing optical device for focusing light from the light source onto the body part; a linear variable filter device for receiving light transmitted through or reflected by the body part and passing light in at least one predetermined narrow wavelength band; and an array detector device for receiving and detecting light from the linear variable filter device.

In accordance with certain embodiments of the wireless spectrometer may include at least one linear variable filter moved by a motor or a piezoelectric bimorph relative to a light source, such that the body part is irradiated with radiation in at least one specified band of wavelengths corresponding to the position of said at least one linear variable filter relative to said light source. In accordance with other aspects of this embodiment, the at least one variable filter includes a plurality of variable filters, and the detector includes a plurality of individual detectors, each of the plurality of variable filters passes light in a different band of wavelengths, each of the plurality of variable filters being associated with a corresponding one of the plurality of detectors.

The remote wireless non-invasive spectral device may be located at the patient's home. Moreover, the remote wireless non-invasive spectral device may be portable, and may be handheld.

The remote invasive device may take a blood sample by a venipuncture, a fingerstick, and a heelstick to generate the constituent value.

The remote invasive device may transmit the constituent value to the remote wireless non-invasive spectral device. The remote wireless non-invasive spectral device may wirelessly transmit information regarding the spectral scan and/or the received constituent value to one or both of the central processing device and a remote processing device. Alternatively, the remote wireless non-invasive spectral device may transmit the information regarding the spectral scan to the remote invasive device, which itself may transmit the received information regarding the spectral scan and/or the constituent value to one or both of the central processing device and the remote processing device. The data may be transmitted over an at least partially wireless transmission path. The transmission path may include one or more of a cellular data link, a telephone modem, a direct satellite link, an Internet link, and an RS232 data connection.

The remote wireless non-invasive spectral device may control administering an amount of a drug to the patient.

### BRIEF DESCRIPTION OF THE DRAWINGS

FIG. 1 is a simplified schematic showing the basic elements of the present invention and the interaction between the same.

FIG. 2A is a basic schematic of transmittance spectrometer, and FIG. 2B is a basic schematic of reflectance spectrometer.

FIG. 3 is a diagram of an instrument detector system used for diffuse reflectance spectroscopy.

FIG. 4 is a schematic representation of diffuse reflectance using an integrating sphere sample presentation geometry.

FIG. 5 is a diagram of a turret-mounted interference filter instrument.

FIG. 6 shows a rotating tilting filter wheel utilizing wedge interference filters.

FIG. 7 shows a spinning filter system in which the light passes through an encoder wheel.

FIG. 8 is a diagram of a grating monochromator spectrometer, with FIG. 8A showing a side view and FIG. 8B a top view of the grating instrument.

FIG. 9 shows a typical predispersive monochromator-based instrument in which the light is dispersed prior to striking the sample.

FIG. 10 shows a post-dispersive monochromator-based instrument in which the light is dispersed after striking the sample.

FIG. 11 illustrates an Acousto Optical Tunable Filter spectrometer.

FIGS. 12A and 12B illustrates a noninvasive near IR spectral device that can be used for obtaining spectral scans.

FIG. 12C illustrates another non-invasive near IR spectral device that can be used for obtaining spectral scans.

FIG. 13A illustrates the blood glucose profile of an elderly diabetic Afro-American female (patient OMF).

FIG. 13B illustrates a modeling equation of near infrared values versus noninvasive glucose values using a Hemo-Cue® system for patient OMF.

FIG. 13C illustrates a comparison of NIR glucose values (dashed line) versus Hemo-Cue® system glucose values (solid line) for patient OMF.

FIG. 14A illustrates the blood glucose profile over five weeks of a middle-age diabetic Caucasian female (patient MLM).

FIG. 14B illustrates a modeling equation of near infrared values versus noninvasive glucose values using a Hemo-Cue® system for patient MLM.

FIG. 14C illustrates a comparison of NIR glucose values (dashed line) versus Hemo-Cue® system glucose values (solid line) for patient MLM.

FIG. 15A illustrates the blood glucose profile over five weeks of a middle-age diabetic Caucasian female (patient SLH).

FIG. 15B illustrates a modeling equation of near infrared values versus noninvasive glucose values using a Hemo-Cue® system for patient SLH.

FIG. 15C illustrates a comparison of NIR glucose values (dashed line) versus Hemo-Cue® system glucose values (solid line) for patient SLH.

FIG. 16A illustrates the blood glucose profile over five weeks of a middle-age diabetic Afro-American male (patient TFD).

FIG. 16B illustrates a modeling equation of near infrared values versus noninvasive glucose values using a Hemo-Cue® system for patient TFD.

FIG. 16C illustrates a comparison of NIR glucose values (dashed line) versus Hemo-Cue® system glucose values (solid line) for patient TFD.

FIG. 17A illustrates the blood glucose profile over five weeks of a young diabetic Caucasian male (patient BDD).

FIG. 17B illustrates a modeling equation of near infrared values versus noninvasive glucose values using a Hemo-Cue® system for patient BDD.

FIG. 17C illustrates a comparison of NIR glucose values (dashed line) versus Hemo-Cue® system glucose values (solid line) for patient BDD.

FIG. 18A illustrates the blood glucose profile over five weeks of a young Caucasian female (patient PFK), used as a control subject.

FIG. 18B illustrates a modeling equation of near infrared values versus noninvasive glucose values using a Hemo-Cue® system for patient PFK.

FIG. 18C illustrates a comparison of NIR glucose values (dashed line) versus Hemo-Cue® system glucose values (solid line) for patient PFK.

FIGS. 19A-B are tables of transform pairs used when data is collected by diffuse reflectance, wherein FIG. 19A depicts the first transform versus second transforms, and FIG. 19B depicts the ratio transform pairs.

FIGS. 20A-B are tables of transform pairs used when data is collected by diffuse-transmittance, wherein FIG. 20A depicts the first transform versus second transforms, and FIG. 20B depicts the ratio transform pairs.

FIGS. 21A-B are tables of transform pairs used when data is collected by clear transmittance, wherein FIG. 21A depicts the first transform versus second transforms, and FIG. 21B depicts the ratio transform pairs.

FIG. 22 is a table of derivative spacing factors.

FIG. 23A shows a schematic representation of an embodiment of a spectrometer in a pre-dispersive configuration.

FIG. 23B illustrates a schematic representation of an embodiment of spectrometer in a post-dispersive configuration.

FIG. 23C illustrates a schematic representation of an embodiment of a spectrometer in a configuration that uses a monochromatic source of light and no filter.

FIG. 23D illustrates a schematic representation of an embodiment of a spectrometer wherein the light source and detector are configured for a transmittance measurement.

FIG. 23E shows a schematic representation of another embodiment of a spectrometer where the light source and detector are configured for a transmittance measurement.

FIG. 23F shows a schematic representation of an embodiment of a spectrometer wherein the light source and detector are configured for a reflectance measurement.

FIG. 23G shows a schematic representation of an embodiment of a spectrometer in a mode where the processing device is physically connected to spectrometer.

FIG. 24 shows a schematic representation of another embodiment of the present invention.

FIG. 25 shows a schematic representation of an embodiment of the present invention wherein a fiber optic bundle is used as a light source for illuminating multiple positions.

FIG. 26 shows a schematic representation of an embodiment of the present invention wherein in a single detector is interfaced to multiple fiber optic light guides.

FIG. 27 shows a schematic representation of a configuration for transmitting the digital signal to a processor.

FIG. 28 shows a schematic representation of another configuration for transmitting the digital signal to a processor.

FIG. 29 shows a schematic representation of a networking arrangement for transmitting the digital signal in accordance with another embodiment of the present invention.

FIG. 30 shows a schematic representation of another embodiment of a networking arrangement for transmitting the digital signal.

FIG. 31 shows a schematic representation of a networking arrangement for transmitting the digital signal in accordance with yet another embodiment of the present invention.

FIG. 32 shows a schematic representation of still another networking arrangement for transmitting the digital signal.

FIG. 33 shows a schematic representation of a further networking arrangement for transmitting the digital signal.

FIGS. 34A-B show an embodiment of a remote spectrometer for performing spectral scans.

FIGS. 35A-B show embodiments of spectroscopic detector arrangements.

FIG. 36 shows an embodiment of a system according to the present invention for predicting blood glucose values.

FIG. 37 shows in more particular detail the elements of a base connection to the main computer.

FIGS. 38A-B show another embodiment of a remote spectrometer.

FIGS. 39A-B show yet another preferred embodiment of a remote spectrometer.

FIGS. 40A-D show front, top, side and back views, respectively, of a table-top blood monitor device according to an embodiment of the present invention.

### DETAILED DESCRIPTION OF PREFERRED EMBODIMENTS

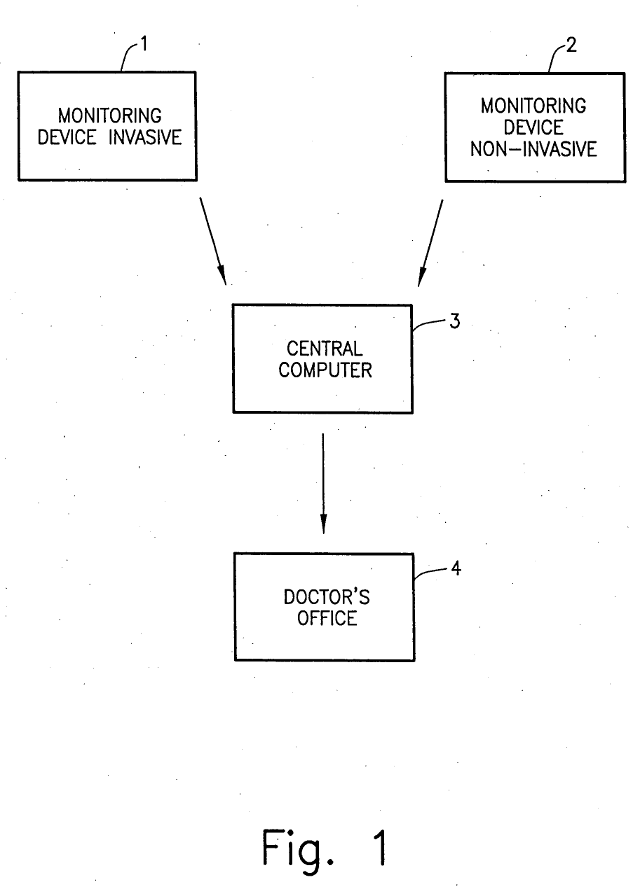

In accordance with a preferred embodiment of the present invention, a system for non-invasive monitoring of blood glucose levels to create an individualized blood glucose profile is provided, wherein a patient can accurately predict the current status of his/her blood glucose levels and obtain immediate feedback on any corrective measures needed in the maintenance thereof. FIG. 1 sets forth the preferred interconnection between the various parts of a preferred embodiment of the present invention. A remote communication link is provided between a conventional invasive blood glucose monitoring device 1 (e.g., Accu-chek® blood glucose monitor or any other electrochemical analytical instrument), a non-invasive spectroscopic device 2 and a central computer 3. In certain embodiments, a further remote communication link is provided between the central computer 3 and the primary doctor's office or hospital 4. The central computer stores the spectral scan from noninvasive device 2, the data obtained using invasive blood glucose monitor 1 and a modeling equation for each individual patient.

Initially, measurements of a patient's blood glucose levels are taken at predetermined intervals over a predetermined period of time using both the spectral device 2 and conventional invasive glucose monitoring methods. Intervals and sampling times as well as monitoring methods are well known to those of skill in the art. See, for example, Tietz, Norbert, Fundamentals of Clinical Chemistry (1976) Saunders Company, Philadelphia, Pa., pages 244-263. For each sample, one or more constituent values are measured by an invasive blood glucose monitoring method. In this regard, a constituent value is a reference value for blood glucose in the sample, which reference value is measured by an independent measurement technique comprising the use of an invasive method (e.g., Hemo-Cue® device). In this manner, the spectral data obtained by noninvasive means for each sample has associated therewith at least one constituent value for that sample.

The set of spectral scans (with its associated constituent values) is divided into a calibration subset and a validation subset. The calibration subset is selected to represent the variability likely to be encountered in the validation subset.

In accordance with a first embodiment of the present invention, a plurality of data transforms is then applied to the set of spectral scans. Preferably, the transforms are applied singularly and two-at-a-time. The particular transforms that are used and the particular combination pairs that are used are selected based upon the particular method that is being used to analyze the spectral data (e.g. diffuse reflectance, clear transmission, or diffuse transmission as discussed in the detailed description). Preferably, the plurality of transforms applied to the spectral data includes at least a second derivative and a baseline correction.

In accordance with a further embodiment of the present invention, transforms include, but are not limited to the following: performing a normalization of the spectral data, performing a ratio on the spectral data, performing a first derivative on the spectral data, performing a second derivative on the spectral data, performing a multiplicative scatter correction on the spectral data, and performing smoothing transforms on the spectral data. In this regard, it should be noted that both the normalization transform and the multiplicative scatter correction transform also inherently perform baseline corrections.

In accordance with a particularly preferred embodiment, the transforms are defined as follows:

The term NULL transform is defined, for the purposes of the present invention, as making no change to the data as originally collected.

The term NORMALIZ transform is defined, for purposes of the present invention, as a normalization transform (normalization). In accordance with this transform, the mean of each spectrum is subtracted from each wavelength's value for that spectrum, then each wavelength's value is divided by the standard deviation of the entire spectrum. The result is that each transformed spectrum has a mean of zero and a standard deviation of unity.

The term FIRSTDRV transform is defined, for purposes of the present invention, as performing a first derivative in the following manner. An approximation to the first derivative of the spectrum is calculated by taking the first difference between data at nearby wavelengths. A spacing parameter, together with the actual wavelength spacing in the data file, controls how far apart the wavelengths used for this calculation are. Examples of spacing parameters include but are not limited to the values 1, 2, 4, 6, 9, 12, 15, 18, 21 and 25. A spacing value of 1 (unity) causes adjacent wavelengths to be used for the calculation. The resulting value of the derivative is assumed to correspond to a wavelength halfway between the two wavelengths used in the computation. Since derivatives of wavelengths too near the ends of the spectrum cannot be computed, the spectrum is truncated to eliminate those wavelengths. If, as a result of wavelength editing or a prior data transform there is insufficient data in a given range to compute the derivative, then that range is eliminated from the output data. Preferably, the value of the spacing parameter is varied such that a FIRSTDRV transform includes a plurality of transforms, each having a different spacing parameter value.

The term SECNDDRV transform is defined, for purposes of the present invention, as performing a second derivative by taking the second difference (i.e., the difference between data at nearby wavelengths of the FIRSTDRV) as an approximation to the second derivative. The spacing parameters, truncation and other considerations described above with regard to the FIRSTDRV apply equally to the SECNDDRV. The second derivative preferably includes variable spacing parameters.

The term MULTSCAT transform is defined, for purposes of the present invention, as Multiplicative Scatter Correction. In accordance with this transform, spectra are rotated relative to each other by the effect of particle size on scattering. This is achieved for the spectrum of the i'th sample by fitting using a least squares equation
Y_{iw}=aᵢ+bᵢm_{w}w=1, . . . , p
where y_{iw} is the log 1/R value or a transform of the log (1/R) value for the i'th sample at the w'th of p wavelengths and m_{w} is the mean log 1/R value at wavelength w for all samples in the calibration set. If Multiplicative Scatter Correction (MSC) is applied to the spectra in the calibration set, then it should also be applied to future samples before using their spectral data in the modeling equation. It is the mean spectrum for the calibration set that continues to provide the standard to which spectra are fitted. The MSC may be applied to correction for log 1/R spectra or Kubelka-Munk data for example. See, Osborne, B. G., Fearn, T. and Hindle, P. H., Practical NIR Spectroscopy, With Applications in Food and Beverage Analysis (2^{nd} edition, Longman Scientific and Technical) (1993).

The term SMOOTHNG transform is defined, for purposes of the present invention, as a smoothing transform that averages together the spectral data at several contiguous wavelengths in order to reduce the noise content of the spectra. A smoothing parameter specifies how many data points in the spectra are averaged together. Examples of values for smoothing parameters include but are not limited to values of 2, 4, 8, 16 and 32. A smoothing value of 2 causes two adjacent wavelengths to be averaged together, and the resulting value of the smoothed data is assumed to correspond to a wavelength halfway between the two end wavelengths used in the computation. Since wavelengths too near the ends of the spectrum cannot be computed, the spectrum is truncated to eliminate those wavelengths. If, as a result of wavelength editing or a prior data transform, there is insufficient data in a given range to compute the smoothed value, then that range is eliminated from the output data. Preferably, the smoothing parameter value is varied such that a smoothing transform includes a plurality of smoothing transforms, each having a different smoothing parameter.

The term RATIO transform is defined, for purposes of the present invention, as a transform that divides a numerator by a denominator. The data to be used for numerator and denominator are separately and independently transformed. Neither numerator or denominator may itself be a ratio transform, but any other transform is permitted.

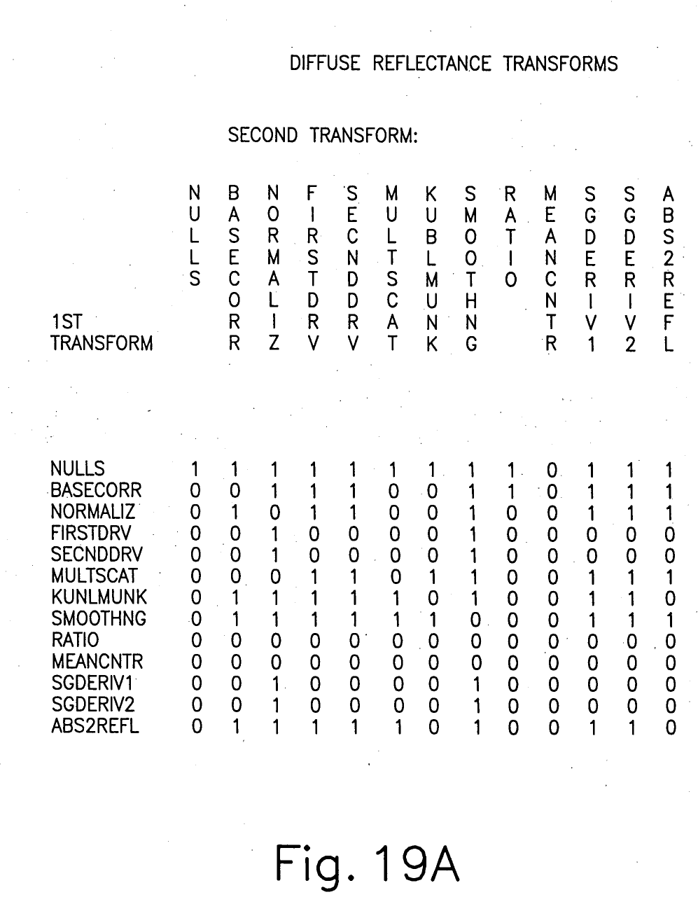

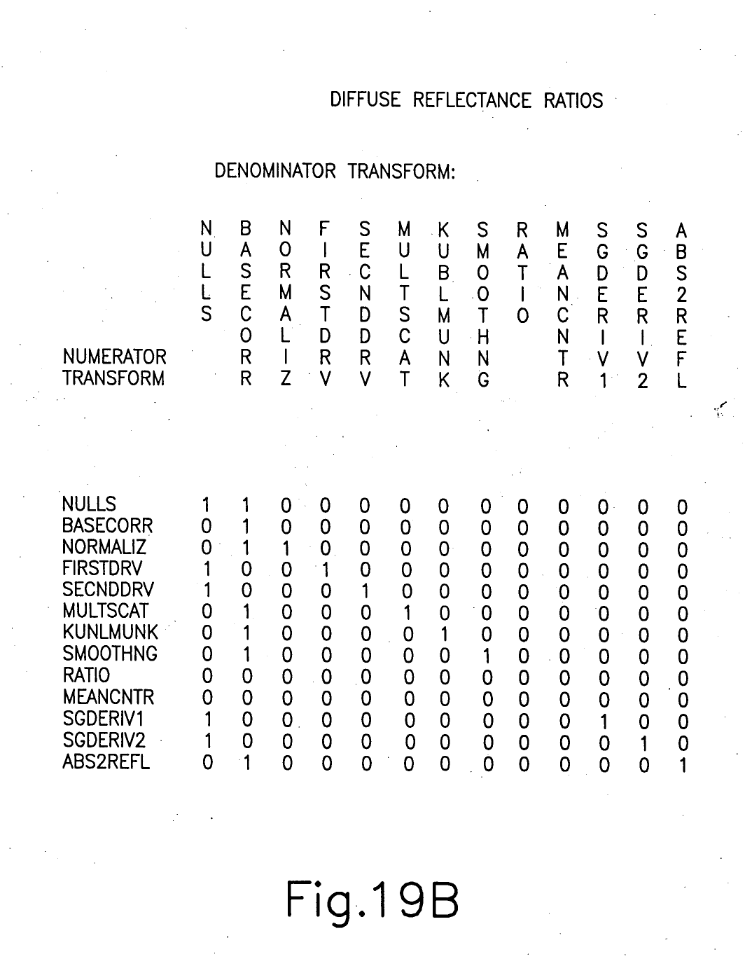

In any event, exemplary transform pairs that can be performed during the automatic search are described in FIGS. 19A (diffuse reflectance), 20A (diffuse transmittance) and 21A (clear transmittance). It should be noted that when “NULL” is selected for both transforms, the original data is used unchanged. The format of the original data is assumed by the system to be absorbency data (i.e., log 1/T or log 1/R).

In addition, exemplary combinations of transforms that can be used for the RATIO transform are illustrated in FIGS. 19B (diffuse reflectance), 20B (diffuse transmittance) and 21B (clear transmittance). If a ratio transform is specified, then numerator and denominator data sets are transformed individually.

In any event, one or more algorithms are then performed on the transformed and untransformed (i.e., Null transform) calibration data sets to obtain corresponding modeling equations for predicting the amount of blood glucose in a sample. Preferably, the algorithms include at least a multiple linear regression analysis (MLR calculations may, for example, be performed using software from The Near Infrared Research Corporation, 21 Terrace Avenue, Suffern, N.Y. 10901) and, most preferably, a Partial Least Squares and Principal Component Analysis as well.

The modeling equations are ranked to select a best model for analyzing the spectral data. In this regard, for each sample in the validation subset, the system determines, for each modeling equation, how closely the value returned by the modeling equation is to the constituent value(s) for the sample. The best modeling equation is the modeling equation that across all of the samples in the validation subset, returned the closest values to the constituent values: i.e., the modeling equation that provided the best correlation to the constituent values. Preferably, the values are ranked according to a Figure of Merit (described in equations 1 and 2 below).

The FOM is defined as
FOM (without Bias) FOM=\sqrt{(SEE²+2*SEP²)/3} 1
FOM (with Bias) FOM=\sqrt{(SEE²+2*SEP²+W*b²)/(3+W)} 2
where SEE is the Standard Error of Estimate from the calculations on the calibration data, SEP is the Standard Error of Estimate from the calculations on the validation data, b is the bias of the validation data (bias being the mean difference between the predicted values and corresponding constituent values for the constituent) and W is a weighting factor for the bias. SEE is the standard deviation, corrected for degrees of freedom, for the residuals due to differences between actual values (which, in this context, are the constituent values) and the NIR predicted values within the calibration set (which, in this context, are the values returned by applying the spectral data in the calibration subset, which corresponds to the constituent values, to the modeling equation for which the FOM is being calculated). Similarly, SEP is the standard deviation for the residuals due to differences between actual values (which, in this context, are the constituent values) and the NIR predicted values outside the calibration set (which, in this context, are the values returned by applying the spectral data in the validation subset, which corresponds to the constituent values, to the modeling equation for which the FOM is being calculated).

The above referenced method of generating a best modeling equation is described in more detail in co-pending U.S. application Ser. No. 09/636,041, filed Aug. 10, 2000, which is incorporated by reference.

The best modeling equation is stored on the central computer, where this modeling equation is used to relate future noninvasive spectroscopic readings to a blood glucose level. Specifically, when the patient acquires a spectral scan using the remote noninvasive spectral device, the spectral scan is transmitted to the central computer where the modeling equation obtained for the individual patient is used to predict the blood glucose level from the spectral scan. If the spectral scan falls within the range of the modeling equation, the blood glucose level is output to the patient. If the spectral scan falls outside the range of the modeling equation, this is an indication that regeneration of the model is needed, and the patient is instructed to recalibrate the system by taking a number of spectral scans using the remote noninvasive spectral device and simultaneously taking a number of invasive measurements of the blood glucose level. The data obtained from the invasive and noninvasive techniques are transferred to the central computer, where a qualified technician supervises the reconstruction of the modeling equation based on the existing and new data points. Preferably, the central computer allows for much of this task to be automated in the manner described above.

Although the invasive blood glucose monitor and remote spectral device may be separate units capable of communicating with the central computer, preferably the invasive blood glucose monitor is capable of communicating the data obtained from the invasive patient blood samples to the remote spectral device, which in turn forwards this information to the central computer. Alternatively, the spectral data may be communicated to the invasive blood glucose monitor which in turn forwards this information onto the central computer. Information from both the spectral unit and invasive unit can be transmitted via any conventional mode of communication (e.g., a cellular data link, a telephone connection, a direct satellite link or an Internet link) to the central computer for analysis. Preferably, the remote spectral device is directly linked to the invasive blood glucose monitor by an appropriate data connection.

More preferably, the remote spectral device has a communication port, such as an RS232 communication port, that is connected to the invasive blood glucose monitor (e.g., an Accu-Check® blood glucose monitor). This allows constituent values from the invasive blood-monitoring device to be loaded directly onto the spectral sensing device.

In one embodiment, the invasive blood glucose monitor and remote spectral device, whether separate units or contained together in a single unit, are interfaced with a remote computer capable of communication with the central computer, for example a desktop workstation, laptop or a hand-held computer such as a PALM PILOT™. Communication between the invasive blood glucose monitor, the remote spectral device, the remote computer and the central computer can be implemented by any known mode of communication. Preferably, both the remote spectral device and the invasive blood glucose monitor have communication ports (such as a RS 232 port) that connect to the remote computer.

In another embodiment, the invasive blood glucose monitor and remote spectral device are contained within a single unit, preferably a portable unit containing a microprocessor and an associated communications interface for communicating with the central computer (similar in design to a PALM PILOT™ hand-held computer). Alternatively, the portable unit may be configured to communicate with a remote computer that, in turn, communicates with the central computer.

The portable unit or remote computer is preferably capable of receiving additional information from the patient for submission to the central computer via the transmission methods identified above. For example, it may be desirable to transmit further information from the patient such as a temperature, blood pressure, heart rate, patient exercise regimen or dietary regimen. In certain embodiments, the portable unit or remote computer is capable of storing the modeling equation and of performing the calculation of the glucose concentration information using the spectral data.

For an initial calibration of the system, any conventional method of invasive blood glucose monitoring may be used in conjunction with spectral scans obtained using the remote spectral device. For example, testing may be conducted in a doctor's office or hospital setting using venipuncture to withdraw blood from the patient at predetermined intervals. Such techniques can be accomplished with such commercially available instruments as the Diasensor 1000®, Corning Express 550®, Roche Cobas Mira® and Ektachem (R)DT60II Analyzer®. More preferably, blood measurements are obtained by the patient using a suitable self monitoring invasive glucose monitor, such as an Accu-Check® blood glucose monitor, a Yellow Springs Instrument® or a Hemo-Cue® blood glucose monitor. Generally, these invasive glucose monitors are electrochemical detectors that monitor reaction of glucose oxidase with glucose in the blood. The current or voltage is measured, and resulting data is displayed as concentration, typically in milligrams per deciliter (mg/dL). To use such a monitor, the patient draws blood from a finger tip using a lancet and places the blood on a chemical test strip that is then inserted into the monitor for analysis. Next, the instrument measures the glucose level in the blood and digitally displays the glucose level.

For noninvasive spectral scans conducted both for an initial calibration of the system and for regular monitoring of blood glucose levels, the remote spectral device is preferably attached to a body part, such as a finger, ear lobe, base of the thumb or other area of the body for which a diffuse reflectance scan in the spectral region of 500 nm to 3000 nm is taken. In certain preferred embodiments, the body part to be tested is the palm of the person's hand or the sole of the foot. It is thought that these body parts, which are less often subjected to direct sunlight and therefore tend to have fewer signs of sun damage such as freckling and tanning, may thereby provide more accurate results.

In order for the remote spectral device to be initially calibrated and an appropriate modeling equation for a particular patient to be obtained, measurements are conducted at predetermined intervals (e.g., morning and evening) over a predetermined period of time (e.g., 4-6 weeks) using both the remote noninvasive spectral device and the invasive blood glucose monitor. For example, in a suitable calibration schedule, the patient would obtain readings from both the remote spectral device and from the invasive blood glucose monitor once a week, over the course of a number of weeks (e.g., a five-week period of time). The information received by virtue of these readings is then forwarded to the central computer for storage and ultimately for use in the calibration of an appropriate algorithm (modeling equation) once sufficient data is received. It is also suitable for the calibration to be conducted in a doctor's office or in a hospital setting using the remote spectral device and a suitable invasive means for measuring blood glucose levels, with such information being sent to the central computer for storage of the information and for calculation of the modeling equation.

The modeling equation may be calculated by a scientist but preferably is conducted to large extent by the central computer, with a scientist overseeing and approving the results of the central computer's calculations. Most preferably, the modeling equation is generated by using a plurality of modeling equations that are generated in the manner described above. After a suitable modeling equation for the patient is determined, the equation is downloaded to the remote microprocessor (e.g., a remote computer or a processor that is integrated into the spectral device), where it is stored and used for predicting the blood glucose level of the patient until a new modeling equation becomes necessary (e.g., after the onset of a disease that affects blood glucose levels or at specified intervals). In other embodiments, the modeling equation is stored only in the central computer, and the spectral scan is merely transmitted to the central computer for analysis. Status checks of the system are conducted on a regular basis (e.g., every two to four weeks). To conduct the status checks, the patient simultaneously collects approximately five spectral scans and invasive blood glucose levels and sends them to the central computer for regeneration of a modeling equation. The five additional data points are added to the existing data, and a new modeling equation is generated using both the new and old data.

The prediction of the blood glucose value based on the spectral data using the algorithm may be conducted at the central computer or may be conducted by a remote computer associated with the remote spectral device or a processor integrated into the spectral device. If the calculation is conducted by the remote computer, the spectral information is nevertheless transmitted to the central computer for evaluation of the algorithm to ensure that recalibration is not needed, and preferably also for evaluation of blood glucose levels.

In certain preferred embodiments, the unit containing the remote spectral device will be able to detect other nonspectral body properties (“non-spectral compensation data”), such as pulse rate, blood pressure and body temperature, that may interfere with the blood glucose spectral prediction. In certain other embodiments, such non-spectral compensation data can be determined separately by the patient and then be input into the unit containing the remote spectral device or the remote computer via a suitable transmission mechanism, such as a key board or voice recognition program. In certain other embodiments, additional information of interest to the diabetic patient and doctor (such as food intake and exercise regimen) may be transmitted via the unit containing the remote spectral device or the remote computer to the central computer. In other embodiments, the nonspectral compensation data may be incorporated into the modeling equations as auxiliary or indicator variables. A discussion of how such variables can incorporated can be found in U.S. patent application Ser. No. 09/636,041.

In certain other embodiments, the remote spectral device may control a drug pump, which can automatically administer the appropriate amount of a drug to the patient such as by means of an IV line or pre-inserted subdermal pump.

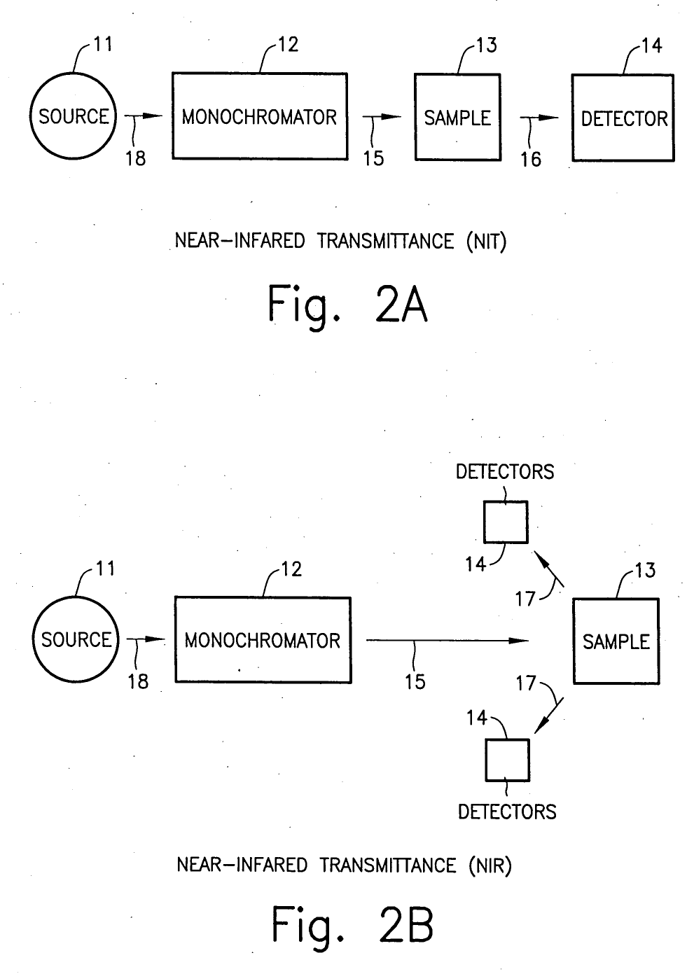

The spectrometers contemplated for use in association with the present invention for use as the noninvasive spectral device can be any of the known versions in the art including, but not limited to, the devices described below and with respect to FIGS. 2-11. For example, spectrometers capable of being integrated into the remote spectral device include filter-type spectrometers, diode array spectrometers, AOTF (Acousto Optical Tunable Filter) spectrometers, grating spectrometers, FT (Fourier transformation) spectrometers, Hadamard transformation spectrometers and scanning dispersive spectrometers. Although the spectral device is preferably a handheld device including both the source of NIR radiation and the detector, larger instrumentation may also be suitable provided that the unit can easily be situated in the user's home and can be transported with the user when necessary. A detailed description of examples of several suitable spectrometers follows.

FIGS. 2A-B show the two most prevalent basic instrument designs common in modern near-infrared analysis: transmittance spectrometers and reflectance spectrometers. FIG. 2A is a basic schematic diagram of a transmittance spectrometer, and FIG. 2B is a basic schematic diagram of a reflectance spectrometer. In both cases, a monochromator 12 produces a light beam 15 having a desired narrow band of wavelengths from light 18 emitted from a light source 11, and the light beam 15 is directed onto a sample 13. However, in the case of a transmittance spectrometer, the detector(s) 14 are positioned to detect the light 16 that is transmitted through the sample 13, and, in the case of a reflectance spectrometer, the detector(s) 14 are positioned to detect the light 17 that is reflected off the sample 13. Depending upon its design, a spectrometer may or may not be used as both a transmittance and a reflectance spectrometer.

Reflectance measurements penetrate only 1-4 mm of the front surface of ground samples. This small penetration of energy into a sample brings about greater variation when measuring nonhomogeneous samples than do transmittance techniques.

The light source utilized in the remote spectral device is preferably a Quartz Tungsten Halogen bulb or an LED (Light Emitting Diode), although any suitable light source, including a conventional light bulb, may be used.

Suitable detectors for use in the analysis of the radiation include silicon (Si), indium/antimony (InSb), indium/gallium/arsenic (InGaAs) and lead sulfide (PbS). In general, lead sulfide detectors are used for measurements in the 1100-2500-nm region, and lead sulfide “sandwiched” with silicon photodiodes are used for visible-near-infrared applications (typically 400-2600 nm).

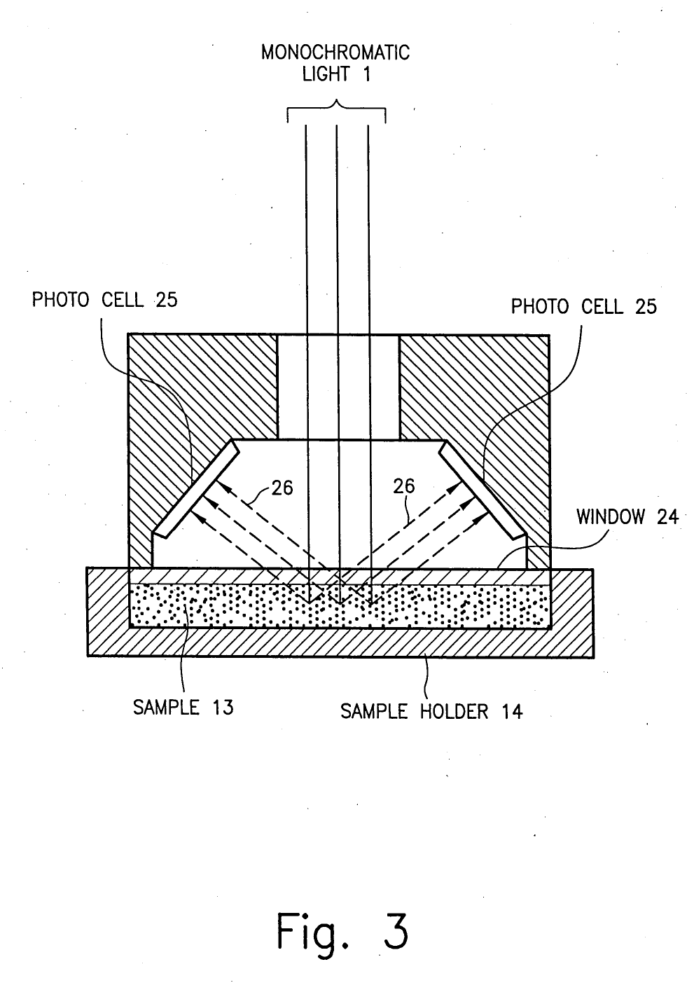

FIG. 3 is a diagram of an instrument detector system used for diffuse reflectance. The geometry of this system provides for monochromatic light 1 to illuminate the sample 13 (i.e., a body part of a patient such as the palm of the hand or the heel of the foot, or another sample that may be held by sample holder 14) at a 90° angle (normal incidence) to the sample. A window 24, through which the monochromatic light can pass, separates sample 13 from the detector. The collection detectors 26 comprise photo cells 25 for detecting the reflected light, each of which is at a 45° angle to window 24. Two or four detectors, each at a 45° angle, can be used.

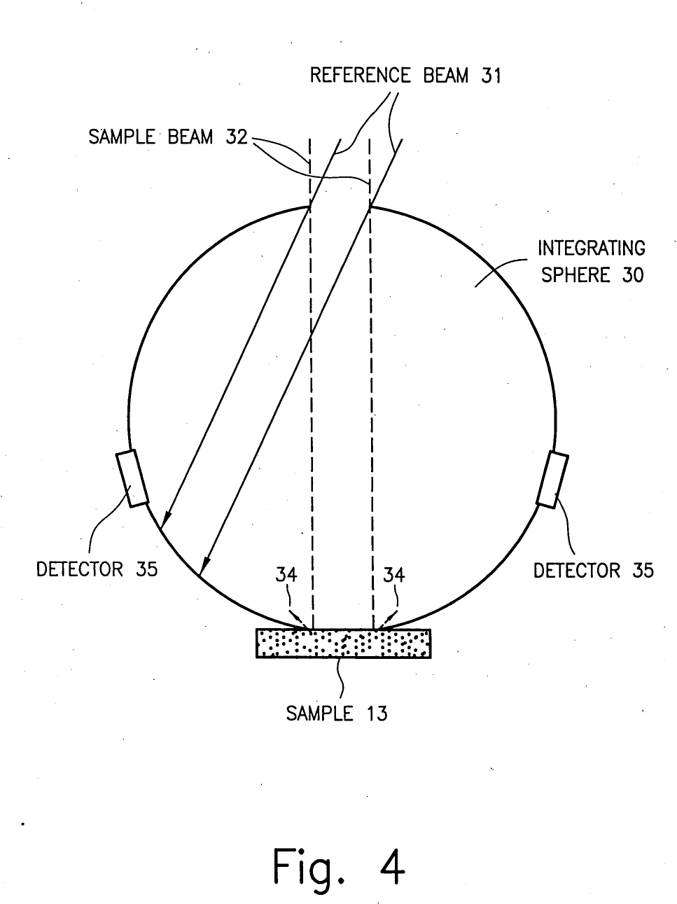

In certain embodiments, the spectrometer may include an integrating sphere such as the one set forth in FIG. 4. In FIG. 4, a schematic representation of diffuse reflectance using an integrating sphere sample presentation geometry is shown. Within the integrating sphere 30 are shown a reference beam 31 and an illuminating beam 32 that hits the sample 13 and is deflected 34 off to the detectors 35. In early spectrometers, “sweet spots” existed on photomultiplier tubes of the detector and early semiconductor and photodiode detectors that made reproducible measurements using detectors very difficult, if not impossible. The integrating sphere cured this problem by protecting the detector from being susceptible to energy fluctuations from the incident beam because of deflection (scattering), refraction or diffraction of light when working in the transmittance mode. In modem applications, the use of the integrating sphere provides for internal photometric referencing, producing a sort of pseudo-double-beam instrument. Single-beam instruments must be set up to measure a reference material before or after the sample scans are taken, requiring inconvenience on the part of the user. For purposes of the present invention, there is no clear-cut advantage of using an integrating sphere over the diffuse reflectance 0-45 geometry. In fact, the 0-45 geometry often lends itself better to a transmittance measurement than do the integrating sphere systems.

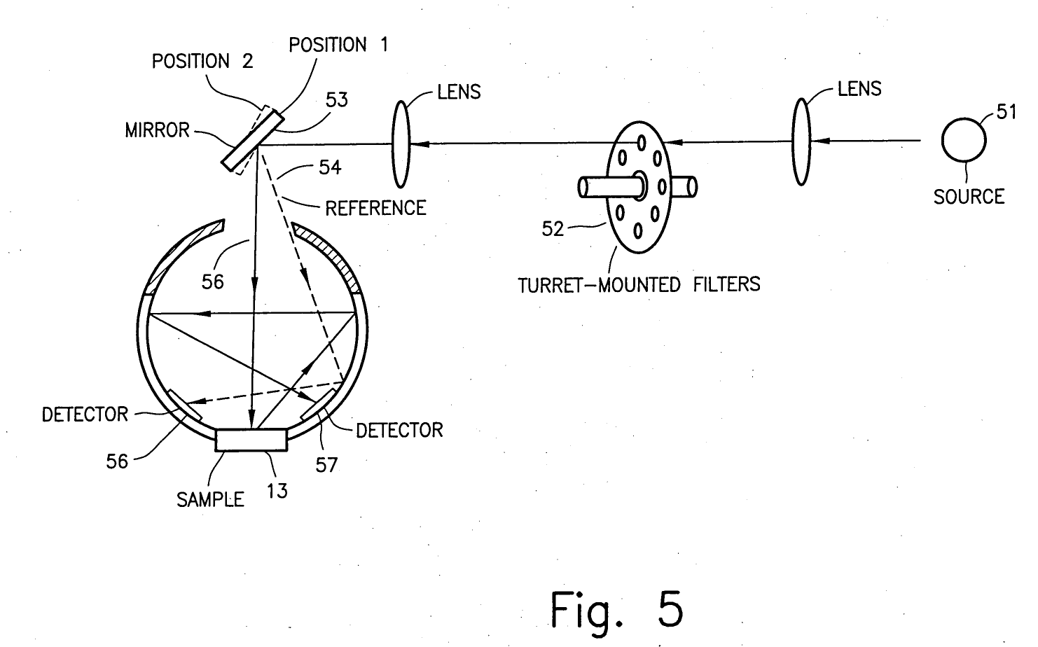

FIG. 5 shows a split-beam spectrometer. Light is transmitted from the light source 51 through the filter 52 (which is shown as being turret-mounted) to a mirror 53 that is positioned to angle the light and create a split-beam, with one resulting beam 54 acting as a reference beam to a first detector 55 and a second resulting beam 56 passing through or reflecting off the sample 13 to a second detector 57. The difference in the amount of detected light at the second detector is compared to the amount of light at the first detector. The difference in the detected light is an indication of the absorbance of the sample.

Nondispersive infrared filter photometers are designed for quantitative analysis of various organic substances. The wavelength selector comprises: a filter, as previously described, to control wavelength selection; a source; and a detector. The instrument is programmed to determine the absorbance of a multicomponent sample at wavelengths and then to compute the concentration of each component.

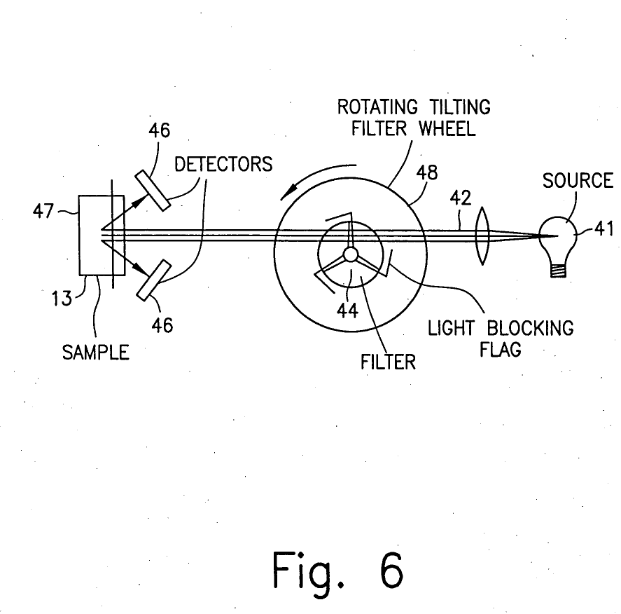

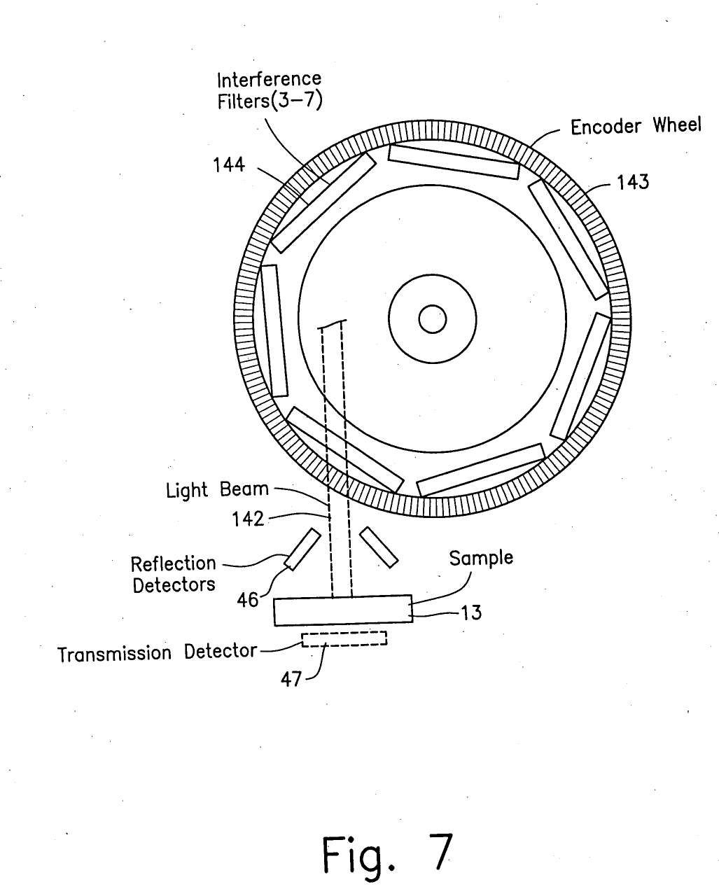

FIGS. 6 and 7 illustrate two basic forms of filter-type NIR spectrometer utilizing a tilting filter arrangement.

FIG. 6 shows a nondispersive infrared filter photometer designed for quantitative analysis of various organic substance. This device utilizes a light source 41, such as the conventional light bulb shown in the figure, to illuminate 42 a rotating opaque wheel 48, wherein the disk includes a number of narrow bandpass optical filters 44. The wheel is then rotated so that each of the narrow bandpass filters passes between the light source and a sample 13. The wheel 48 controls which optical filter 44 is presently before the light source. The filters 44 filter the light from the light source 41 so that only a narrow selected wavelength range passes through the filter to the sample 13. Optical detectors 46 are positioned to detect light that either is reflected by the sample (to obtain a reflectance spectra, as illustrated with detectors 46) or is transmitted through the sample (to generate a transmittance spectra, as illustrated with detector 47). The amount of detected light is then measured, thereby providing an indication of the amount of absorbance of the light by the substance under analysis.

FIG. 7 shows a rotating encoder wheel 143 utilizing wedge interference filters 144 for blocking light. Light 142 is transmitted through the encoder wheel 143 at varying wavelengths and bandpass, dependent on the incident angle of the light passing through the interference filter 144 to the sample 13. Optical detectors 46 are positioned to detect light that either is reflected by the sample (to obtain a reflectance spectra, as illustrated with detectors 46) or is transmitted through the sample (to generate a transmittance spectra, as illustrated with detector 47). The amount of detected light is then measured, providing an indication of the amount of absorbance of the light by the substance under analysis.

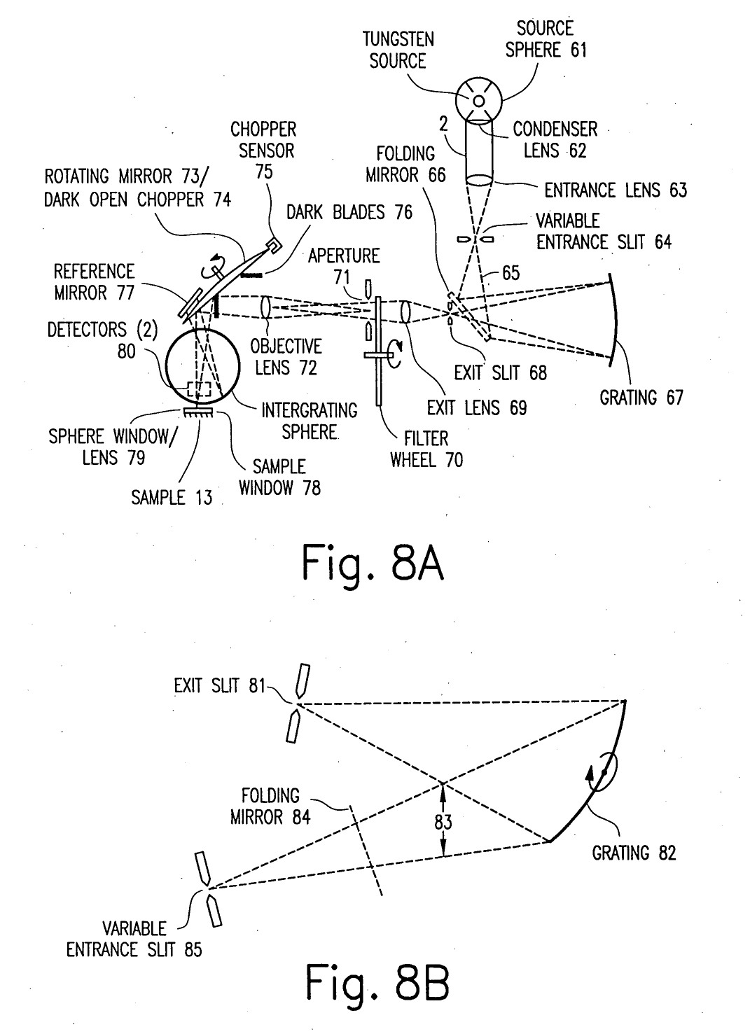

FIGS. 8A and 8B illustrate a grating monochrometer. In FIG. 8A, light is transmitted from a source 61 containing a condenser lens 62 through an entrance lens 63 to a variable entrance slit 64 where the beams of light 65 are deflected to a folding mirror 66. The mirror sends the beam of light to a grating 67, which in turn projects the light through an exit slit 68 to an exit lens 69. The light then passes through a filter wheel 70 containing aperatures 71 to an objective lens 72 and then on to a rotating mirror 73. The rotating mirror 73 has a dark/open chopper 74, a chopper sensor 75, a dark blade(s) 76 and a reference mirror 77 capable of sending a reference beam. The light is transmitted from the rotating mirror through a sphere window lens 79 and a sample window 78 to the sample 13, which then reflects the light to detector(s) 80. In FIG. 8(B) a top view of the grating instrument is shown, wherein light passes through the exit slit 81 to the grating 82 which projects the beam 83 to a folding mirror 84, from which it is projected to a variable entrance slit 85.

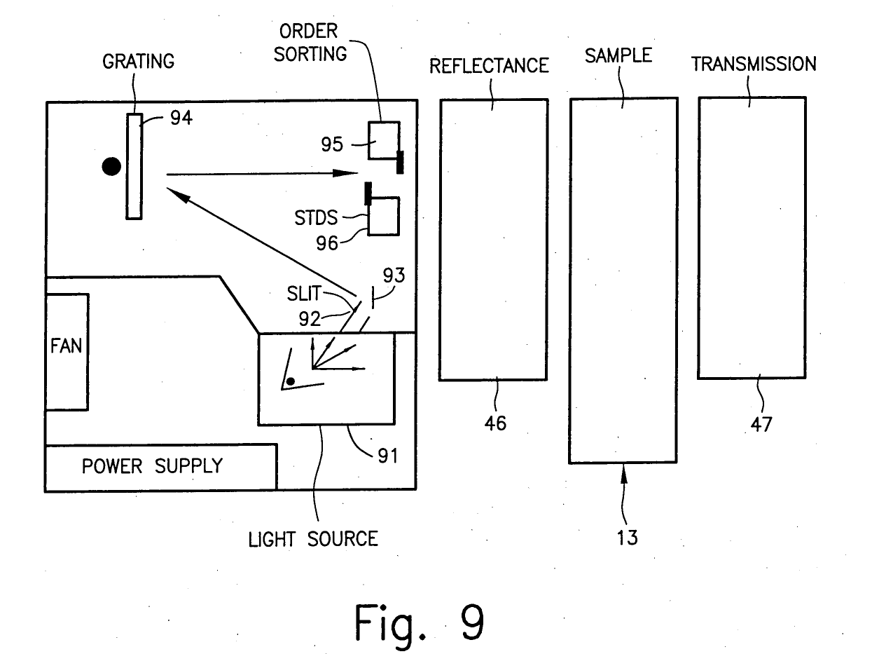

FIG. 9 shows a schematic diagram of typical pre-dispersive monochromator-based instrument in which the light is dispersed prior to striking the sample. As shown in FIG. 9, the light source 91 transmits a beam of light 92 through an entrance slit 93 and onto a grating 94. The grating 94 separates the light into a plurality of beams of different wavelengths. Via the order sorting 95 and stds 96 components, a desired band of wavelengths is selected for transmission to the sample 13. As illustrated, this spectrometer may also be used with both transmittance detectors and reflectance detectors 46.

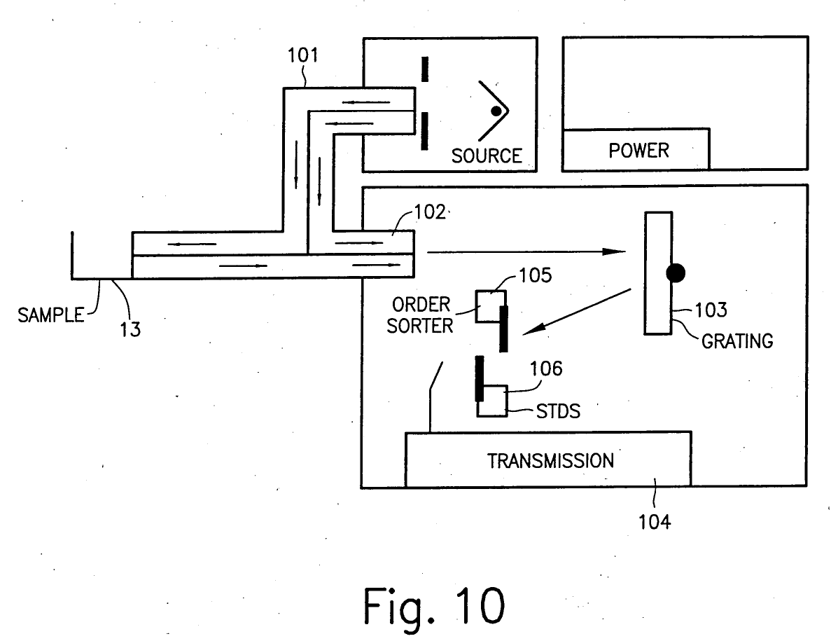

FIG. 10 shows a schematic diagram of a typical post-dispersive monochromator. This type of instrument provides the advantage of allowing the transmission of more energy on the sample via either a single fiberoptic strand or a fiberoptic bundle. Referring to FIG. 10, white light is piped through the fiberoptic strand or fiberoptic bundle 101 and onto the sample 13. The light is then reflected 102 off the sample 13 and back to the grating 103 (the dispersive element). After striking the grating 103, the light is separated into the various wavelengths by order sorting 105 and stds 106 components prior to striking a detector 104. The post-dispersive monochromator can be used with reflectance detectors.

The dedicated dispersive (grating-type) scanning NIR instruments, like those described above, vary in optical design but generally have the common features of tungsten-halogen source lamps, single monochromator with a holographic diffraction grating, and uncooled lead sulfide detectors.

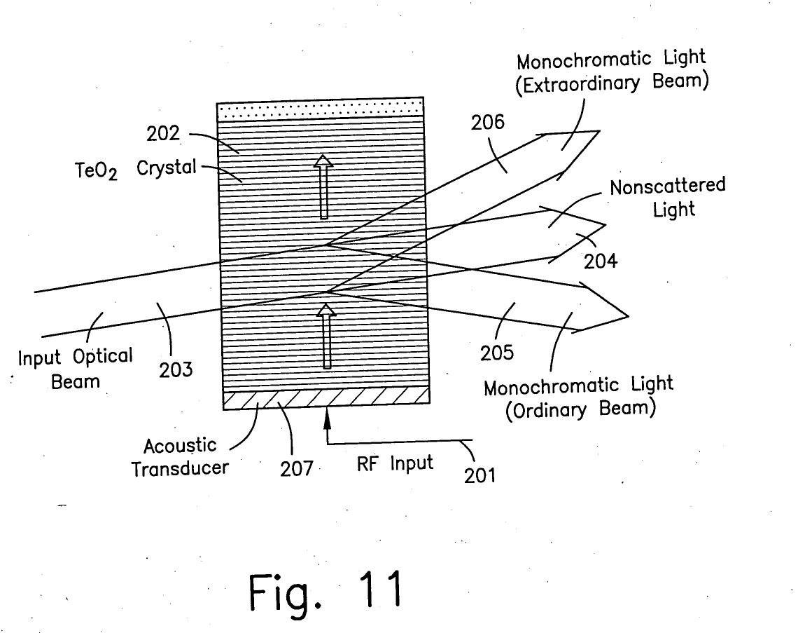

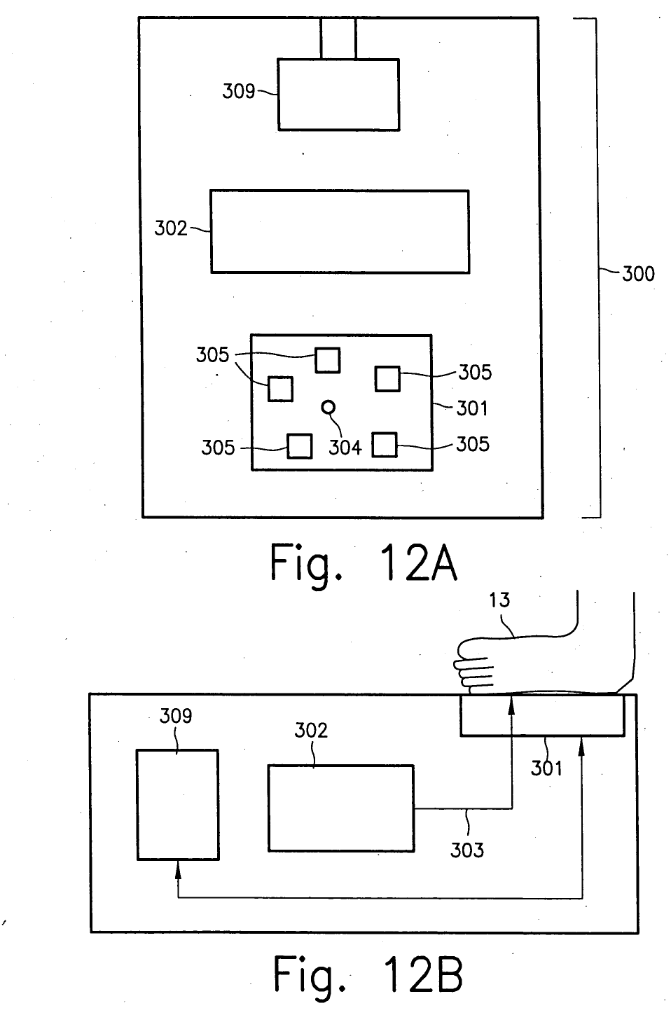

FIG. 11 depicts an Acousto Optical Tunable Filter spectrometer utilizing an RF signal 201 to generate acoustic waves in a TeO₂ crystal 202. A light source 203 transmits a beam of light through the crystal 202, and the interaction between crystal 202 and RF signal 201 splits the beam of light into three beams: a center beam of unaltered white light 204 and two beams of monochromatic 205 and orthogonally 206 polarized light. A sample 13 is placed in the path of one of the monochromatic beams. The wavelength of the light source is incremented across a wavelength band of interest by varying the RF frequency.

On one surface of the specially cut crystal an acoustic transducer 207 is bonded. The acoustic transducer is a piezoelectric material, such as LiNbO₃ driven by 1-4 W of radio frequency (RF) coupled into the transducer. The high-frequency (30-200 MHz) acoustic waves induce index of refraction waves in the acoustooptical material. The waves travel through the crystal very quickly. Typically, within 20-30 μsec the acoustic waves “fill” the crystal, interacting with the broad-band light traveling through the crystal. The angles of the crystal axis, the relative angles of the broad-band light into three beams. As noted above, the center beam is the unaltered white light traveling through the crystal. The TeO₂ material has virtually no absorption from the visible spectrum all the way to about 5 μm. The two new beams generated by the acoustically excited crystal are, as discussed above, monochromatic and orthogonally polarized. These beams are used as monochromatic light sources for analytical purposes.

The main advantage of the AOTF optics is that the wavelength is electronically selected without the delays associated with mechanical monochromators. The electronic wavelength selection allows a very high-duty cycle because almost no time is wasted between wavelength switching. In comparison with “fast-scanning” instruments, the advantage is not only that the scanning rate is orders of magnitude faster but also that the wavelength access is random. If only four or five selected wavelengths are required for the concentration equation, the AOTF instrument is able to select those and is not confined to accessing all wavelengths serially (as in fast grating monochromators) or multiplexed (as in FT-NIR).

Besides the speed and efficiency of wavelength selection, the AOTF instruments generally are much smaller than grating monochromators but with equal resolution. In a properly engineered design, the long-term wavelength repeatability also surpasses that of the grating monochromator.

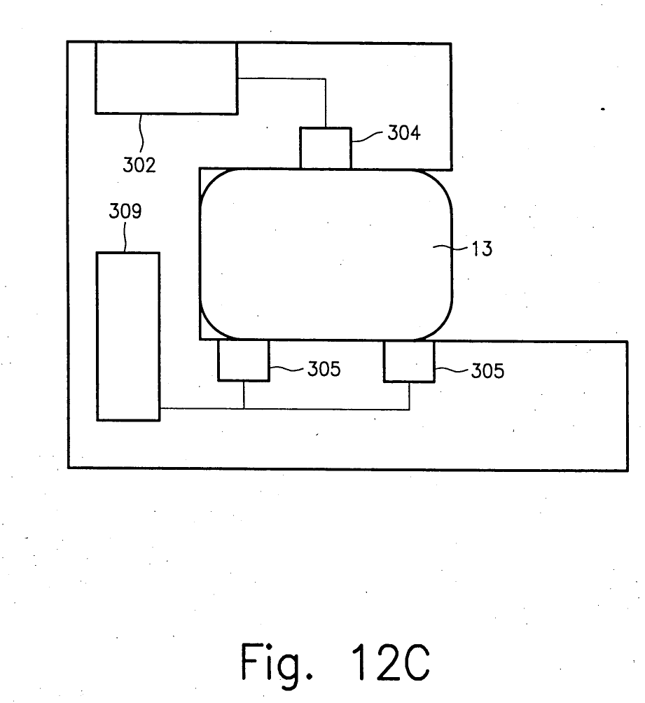

FIGS. 12A and 12B depict a preferred remote spectrometer 300 for performing noninvasive spectral scans of a sample 13 (i.e., the base of the thumb) to predict blood glucose levels. As shown in FIG. 12A, sample portion 301 includes a light emitting portion 304 and a plurality of detectors 305 surrounding light emitting portion 304. As shown in FIG. 12B, light emitting portion 301 of the sample module is connected by a fiber optic cable 303 to the monochrometer 302 comprising a light source and a grating for selecting desired wavelength (e.g., 1100-2500 nm). A communication module 309 receives spectral scans from the detectors 305 in sample portion 301 and transmits the spectral scan data to a remote computer (not shown). The communication module may also be configured to store the spectral scan data for subsequent use.

The spectrometer 300′ of FIG. 12C is similar to the spectrometer 300 of FIGS. 12A and 12B, except that light emitting portion 304 is located above the five detectors 305, and the sample 13 (in this case, the base of the thumb) is placed between light emitting portion 304 and detectors 305.

Once the remote computer obtains the spectral scan, the spectral scan will then be stored in the memory on the computer. The remote computer will then automatically access the central computer, establish a communication link and then upload the spectral scan to the central computer. Alternatively, the remote spectrometer 300 itself may itself include a processor, a memory and a communications port for uploading the spectral data to the central computer.

The central computer is preferably a server or workstation capable of holding spectral databases for a plurality of patients. The workstation is preferably configured to allow multiple clients to concurrently access the server. Any known WAN networking technology may be used to promote this functionality. For each client, the central computer will store: 1) all spectral data collected from that client; 2) all constituent data from that client (from the invasive blood-monitoring device); and 3) the current modeling equation that is being used to predict the blood glucose level from the spectral scan. In preferred embodiments, the central computer also stores non-spectral compensation data and may further store additional information submitted by the patient, such as dietary intake and exercise regimen.

The central computer, in one embodiment, receives a plurality of spectral scans from the spectral device (remote or otherwise) and associated constituent data (invasively-measured blood glucose levels) from an invasive measurement device, and calculates the modeling equation from which future blood glucose levels will be predicted, preferably using the preferred technique discussed above.

The central computer then receives spectral data from a remote spectral device and, if the spectral scan is within the range of the modeling equation, predicts a blood glucose value from the modeling equation and sends the blood glucose value back to the patient. The central computer may also alert the patient when, based on the spectral data, the modeling equation is no longer valid, and either sends a message to the patient to attempt another reading or sends a message to begin a recalibration procedure. The central computer may also instruct the patient to begin a recalibration procedure at regular intervals (e.g., once a month). Once recalibration is initiated, the patient will perform a number of spectral scans and corresponding invasive measurements (to obtain constituent values) at designated times. The spectral data and constituent values are uploaded to the central computer, and the central computer then regenerates the modeling equation based on the original data as well as on the data uploaded during the recalibration procedure. As described above, in some instances it may be necessary to regenerate the modeling equation based only on new data. In this case, the patient will be instructed to take a sufficient number of invasive and noninvasive measurements to obtain a completely new modeling equation. Preferably, the central computer transmits the appropriate timing schedule to the patient via communication with the remote computer or the remote spectrometer 300. In this regard, additional instructions, such as medication schedules, may be transmitted to the patient in the same manner.

In another embodiment, the central computer regenerates a modeling equation for an individual patient, as described above and transmits the modeling equation to the portable unit containing a microprocessor and spectral device (and preferably an acceptable invasive blood glucose monitor). The patient can then conduct further noninvasive testing on a pre-determined schedule, with the portable unit itself predicting the individual's blood glucose using the modeling equation previously downloaded from the central computer. The spectral data can then be subsequently sent to the central computer for analysis. If the spectral data is not within an acceptable parameter a message is sent to the patient to regenerate (i.e., recalibrate) a modeling equation. The determination of whether the data is within acceptable parameters may be made by the portable unit itself, or alternatively by the central computer. Regeneration can also be initiated at regular predetermined intervals (such as monthly). As described above, regeneration may be initiated either partially or fully with new data, depending on the particular situation.

As set forth above, in certain preferred embodiments, the central computer is capable of transmitting basic instructions to the patient to obtain blood glucose levels or to take medication. In further embodiments, more complicated instructions can be sent to the patient, such as instructions to call the patient's doctor for reevaluation of medication or instructions to adjust medication regimen, diet or exercise.

Preferably, the data received by the central processing unit and the data sent back to the remote spectral device is time/date stamped and is secured (e.g., encrypted, requiring a key to decipher, or transmitted over a dedicated line and requiring a password for access).

Preferably, in those embodiments in which the unit containing the remote spectral device (or a remote computer) performs certain data storage and glucose prediction functions, all information obtained during the scanning is nevertheless submitted to the central computer for analysis and to ensure that regeneration of the modeling equation is not necessary.

The operation of the central computer and the maintenance of the models from each patient are preferably overseen by trained staff members.

In certain embodiments, the central computer is further connected to one or more doctor's offices, hospitals or other patient care facilities, such as a nursing home or hospice. This enables communication of relevant information directly from the central computer to the doctor where the information can be monitored and become part of the standard file on a patient. The doctor may contact the central computer to obtain information regarding the blood glucose levels of the relevant patients or can request individual information regarding patients. In a preferred embodiment, the doctor is able to obtain information concerning patient information, such as heart rate, pulse, blood pressure, dietary intake and exercise regimen.

In certain embodiments, blood glucose information is automatically transmitted to the doctor by the central computer upon completion of the central computer's receipt and analysis of a particular patient's information (e.g., STAT samples). In other embodiments, the blood glucose information for all patients in the system is automatically transmitted to the patient's doctor at regular intervals, preferably twice a day. In other preferred embodiments, other relevant patient information, such as heart rate and blood pressure, also are automatically transmitted to the doctor.

In yet a further embodiment, the doctor is capable of transmitting instructions concerning patient care to the central computer, which instructions are both stored by the central computer in the patient's file and transmitted by the central computer to the patient's remote computer as a message.

In a further embodiment of the present invention, the central computer is associated with a website through which the data can be accessed by the patient and/or physician. The website may contain further information relating to disease state, including referral service, articles of interest, links to hospitals and links to diabetes related associations. In further embodiments, diabetes related equipment and supplies can be purchased through the website. In yet further embodiments, the website contains or is linked to a remote licensed pharmacy capable of receiving prescriptions and filling prescriptions.

In certain embodiment, access to a patient's records is obtained through a secure line by entering a predesignated password. In other embodiments, patient information supplemental to blood glucose levels, such as information on exercise and dietary regimen, heart rate and pulse, can be digitally transmitted to the website by modem or by e-mail.

The method of the present invention, although described above in terms of the measurement of blood glucose, can also be used to predict any known clinical chemistry, hematology, or immunology body fluid parameters, including, e.g., insulin levels.

### EXAMPLES

The following non-limiting examples illustrate suitable methods according to the invention. It should be noted that the examples provided below are not meant to be exclusive.

### Example 1

A clinical study of seven patients was conducted over five weeks. Testing occurred once a week, as discussed below. At each time point, a fingerstick was taken and read on a Hemo-Cue® blood glucose analyzer (invasive blood glucose level) to obtain a constituent value, and a near IR reading was taken at the base of the thumb (non-invasive spectral scan used to predict blood glucose level).

For each individual patient, readings were performed at various times in relation to eating. The first reading was taken in the morning following a fast of at least eight hours. Immediately following the taking of the first reading, the patients ate a full meal. The second reading was taken at approximately 30 minutes after eating. Subsequent readings were taken approximately every 30 minutes up to and including 150 minutes after eating for a total of six readings (in some instances, a seventh reading was taken at 180 minutes after eating).

The near IR reading was taken with a model AP 1365-II Rapid Content Analyzer (RCA system), including a Model 6500 scanning Near-Infrared spectrometer. Each patient placed the base of his/her thumb on the Rapid Content Sample Module of the device (which included four detectors disposed about a source of NIR radiation).

The RCA system was manufactured by FOSS-NIR Systems and has the following specifications:

Monochromator System

| Model Number | Wavelength Coverage |
|---|---|
| AP-1365-II | Model 6550 monochromater with 400-2500 nm coverage |

Rapid Conent ™ Analyzer (RCA) Specifications

| | |
|---|---|
| Scan Rate | 1.8 scans/second |
| Wavelength Range | 1100-2500 nm (Standard Mode) |
| | 400-2500 nm (Wide-Range Mode) |
| Wavelength Accuracy | ±0.3 nm (Basis: Polystyrene Standard |
| | peaks 1100-2500 nm) |
| Wavelength Repeatability | ±0.010 nm (Standard Deviation of 10 |
| | consecutive scans 1100-2500 nm) |
| Noise (peak-to-peak) | <3.0 (400-700 nm) < 0.4 (700-2500 nm) |
| Noise (RMS Avg) | <0.2 (400-700 nm) < 0.04 (700-2500 nm) |
| Spectral Bandwidth | 10 ± 1 nm |
| Photometric Range | 3.0 Au (1100-2500 nm) |
| Linearity | ±1.0% |
| (2% to 99% reflectivity or | |
| transmissivity) | |
| Stray Light | <0.1% at 2300 nm |
| Operating Temperature Range | 15° C. to 33° C. (60° F. to 92° F.) |
| Power Consumption | 150 watts (maximum) |

The patients in the clinical study have the following profiles:

Monochromator System

| | |
|---|---|
| Model Number | Wavelength Coverage |
| AP-1365-II | Model 6550 monochromater with 400-2500 nm coverage |

Rapid Conent ™ Analyzer (RCA) Specifications

| | |
|---|---|
| Scan Rate | 1.8 scans/second |
| Wavelength Range | 1100-2500 nm (Standard Mode) |
| | 400-2500 nm (Wide-Range Mode) |
| Wavelength Accuracy | ±0.3 nm (Basis: Polystyrene Standard peaks 1100-2500 nm) |
| Wavelength Repeatability | ±0.010 nm (Standard Deviation of 10 consecutive scans |
| | 1100-2500 nm) |
| Noise (peak-to-peak) | <3.0(400-700 nm) <0.4(700-2500 nm) |
| Noise (RMS Avg) | <0.2(400-700 nm) <0.04(700-2500 nm) |
| Spectral Bandwidth | 10 ± 1 nm |
| Photometric Range | 3.0 Au (1100-2500 nm) |
| Linearity | ±1.0% |
| | (2% to 99% reflectivity or transmissivity) |
| Stray Light | <0.1% at 2300 nm |
| Operating Temperature Range | 15° C. to 33° C. (60° F. to 92° F.) |
| Power Consumption | 150 watts (maximum) |

Patient SLR dropped out of this clinical study for medical reasons that are not relevant here.

Criteria for Evaluation

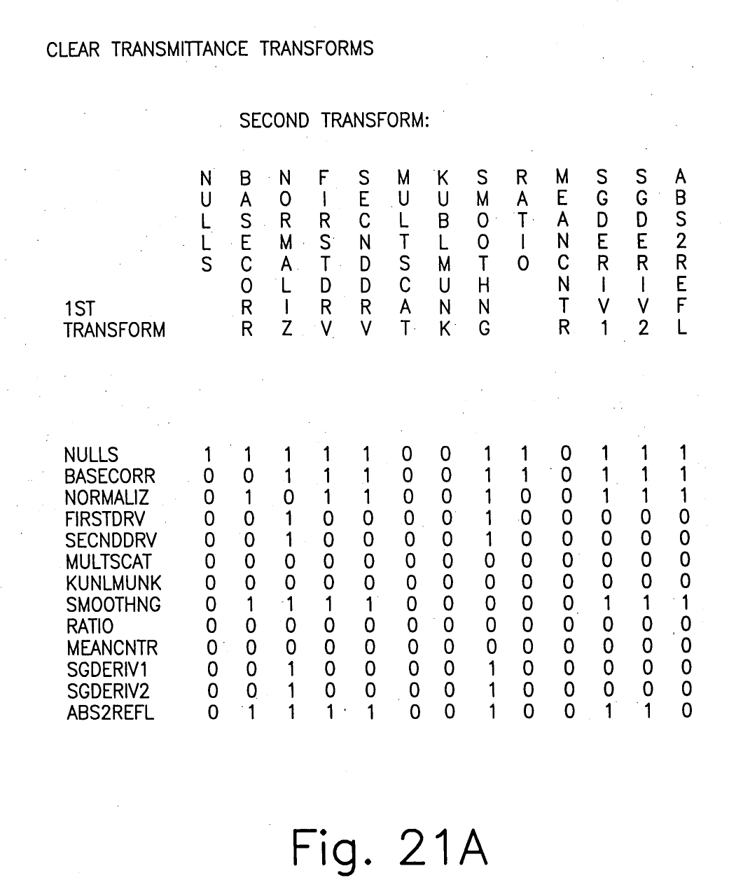

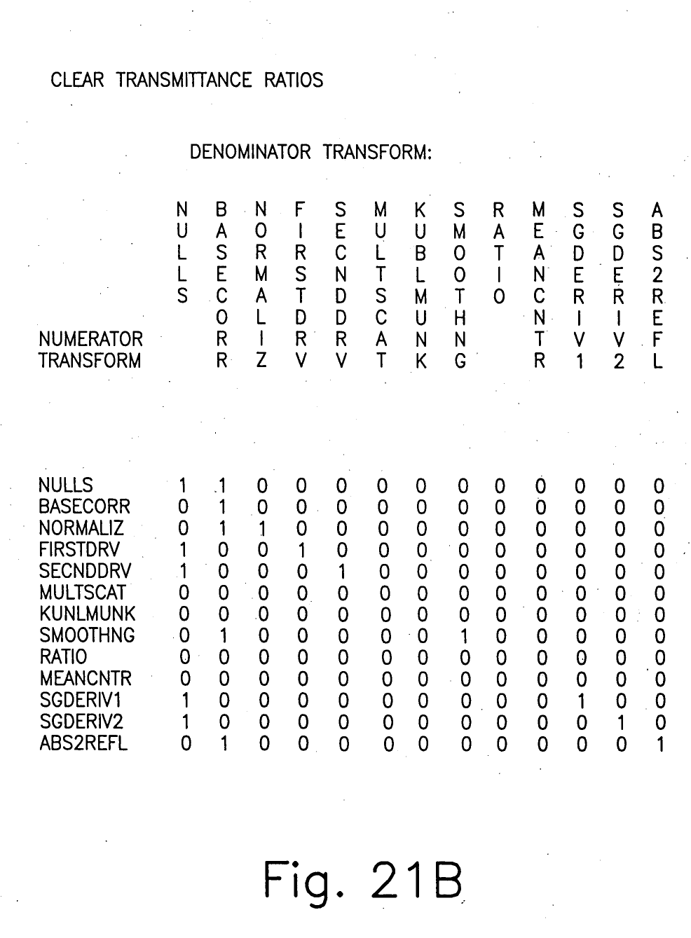

For each patient, the spectral data and associated constituent values were randomly divided into a calibration set and a validation set, with approximately equal numbers of readings in each set. Two successive data transforms were then applied to the spectral data in the calibration and validation sets. The following is a list of the data transforms applied: NULL, ABS2REFL, NORMALIZ, FIRSTDRV, SECNDDRV, MULTSCAT, KUBLMUNK, SMOOTHNG, RATIO, MEANCNTR, SGDERIV1 and SGDERIV2. If a RATIO transform is selected, both numerator and denominator transforms are also selected from this list (except that neither may itself be a ratio). FIGS. 21A,B (clear transmittance) identify the manner in which the transforms were applied, except that the BASECORR transform was not used in the Study.

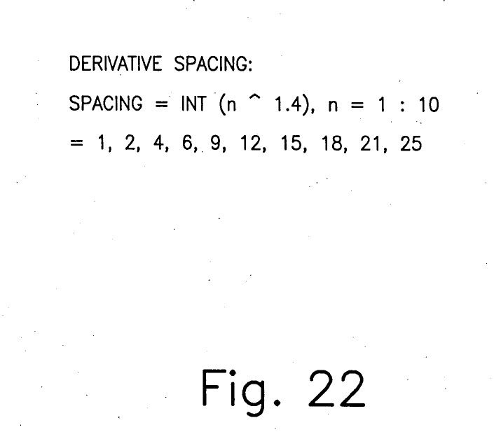

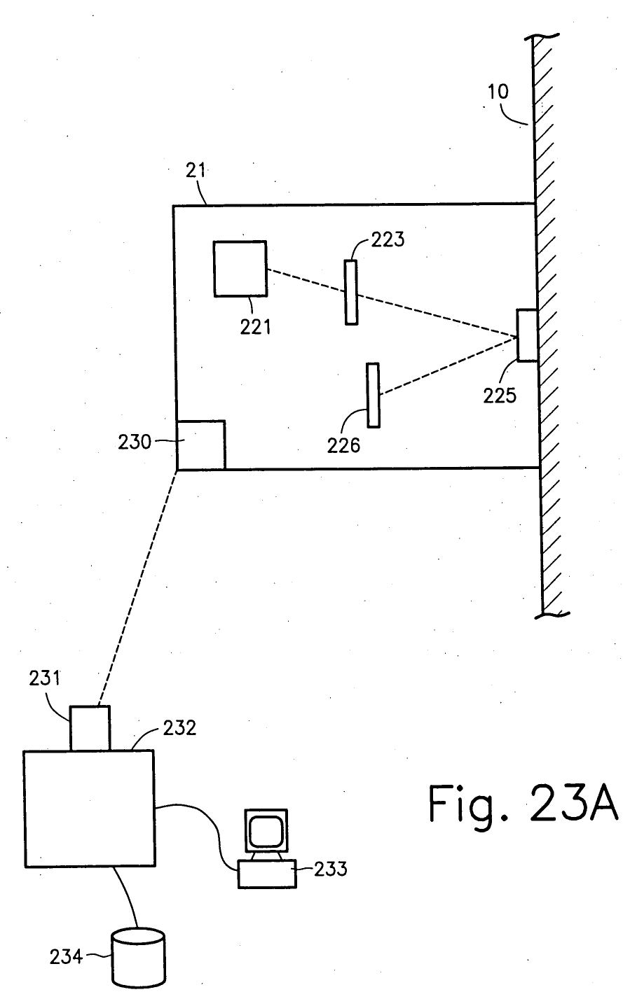

Some of the transforms, particularly the smoothing transform and derivative transforms, have parameters (e.g., spacing of data points for the first derivative, second derivative and smoothing transforms) associated with them. During the automatic search, for any data transform that requires a parameter, the data transform is performed multiple times, using different values of the parameter. The values used for each data transform requiring a parameter are presented in FIG. 22.

The following algorithms were then applied to each set of transformed spectral scan: PLS (partial least squares), PCR (principal component regression), and MLR (multiple linear regression), thereby producing three equations for each set of transformed spectral scan. Each of the equations (or calibration models) is then applied to the spectral data in the validation set.

The “best” modeling equation was selected on the basis of a “Figure of Merit”, which is computed using a weighted sum of the SEE and SEP, the SEP being given twice the weight of the SEE. The FOM was calculated using the following equation, wherein “Bias in FOM” is unchecked:
FOM=\sqrt{(SEE²+2*SEP²)/3}, where:
SEE is the Standard Error of Estimate from the calculations on the calibration data; and SEP is the Standard Error of Estimate from the calculations on the validation data.

When all calculations have been completed, the results are sorted according to the Figure of Merit (FOM), and the equation corresponding to the data transform and algorithm providing the lowest value for the FOM is determined and designated as the best equation.

Results and Discussion

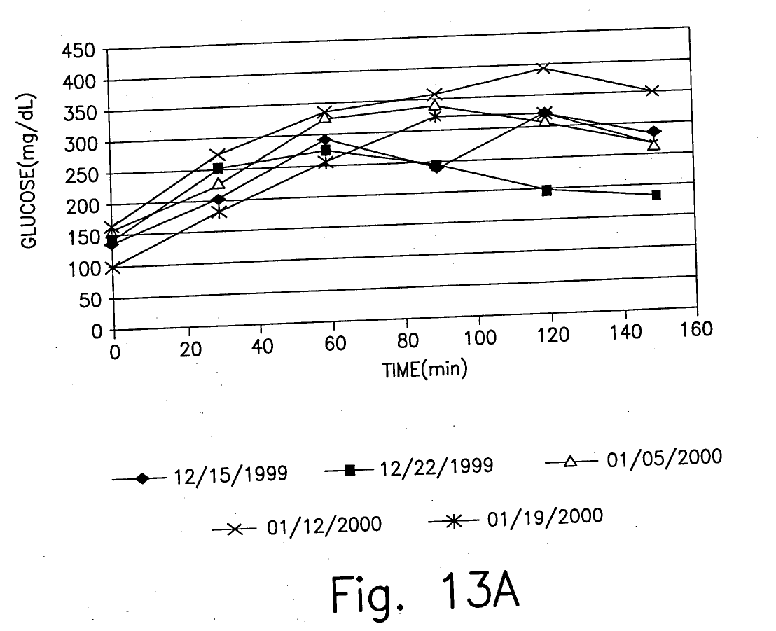

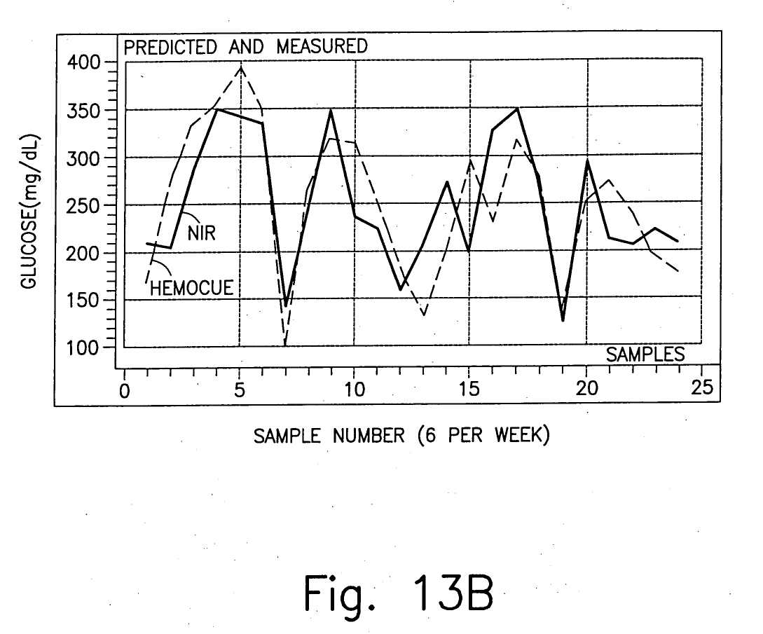

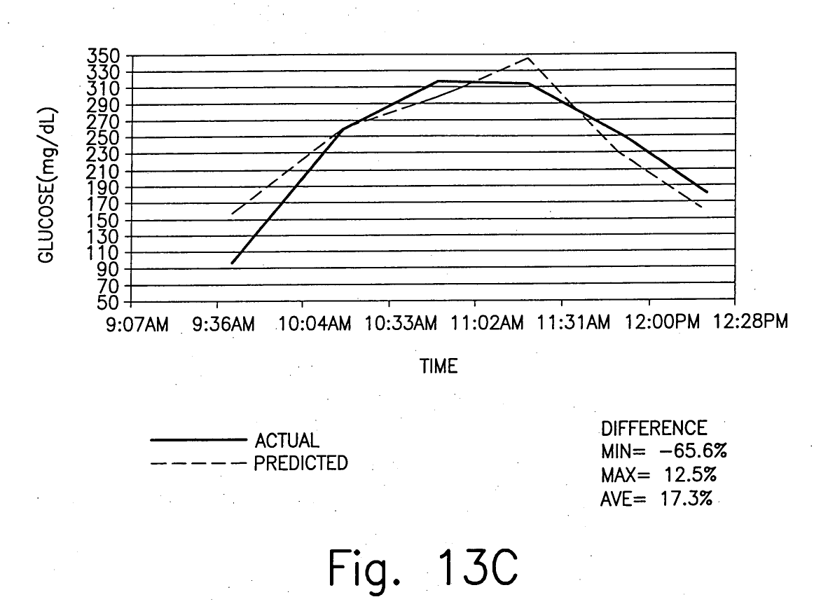

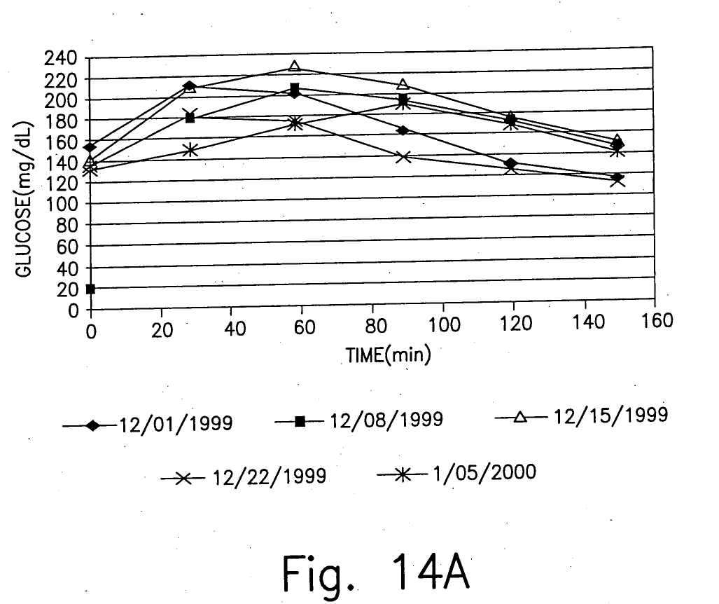

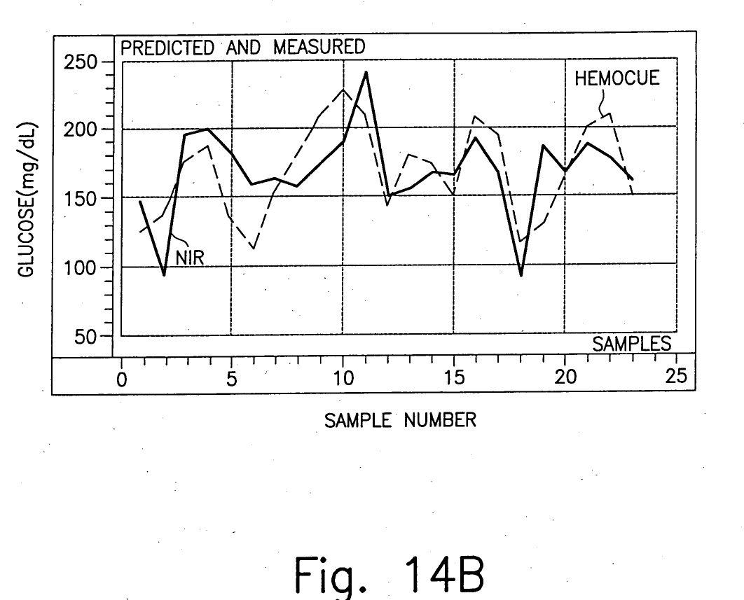

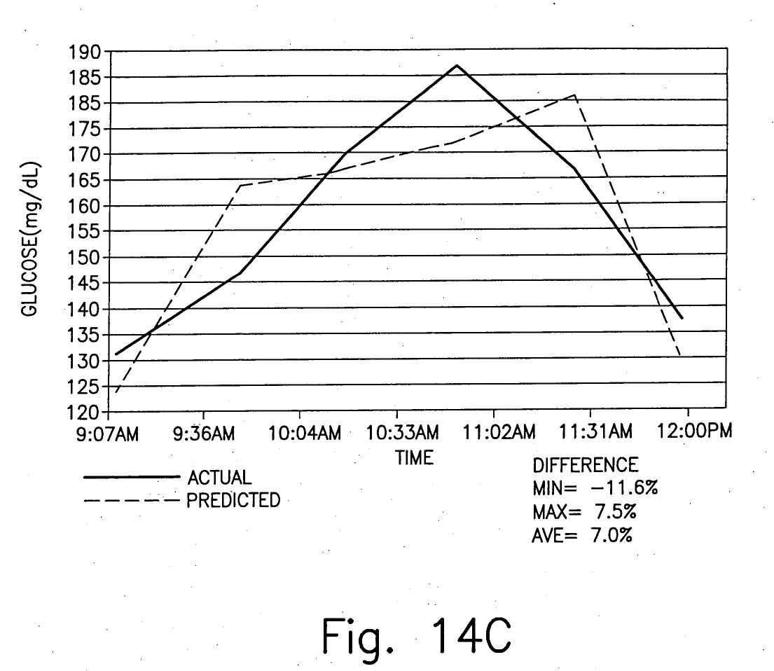

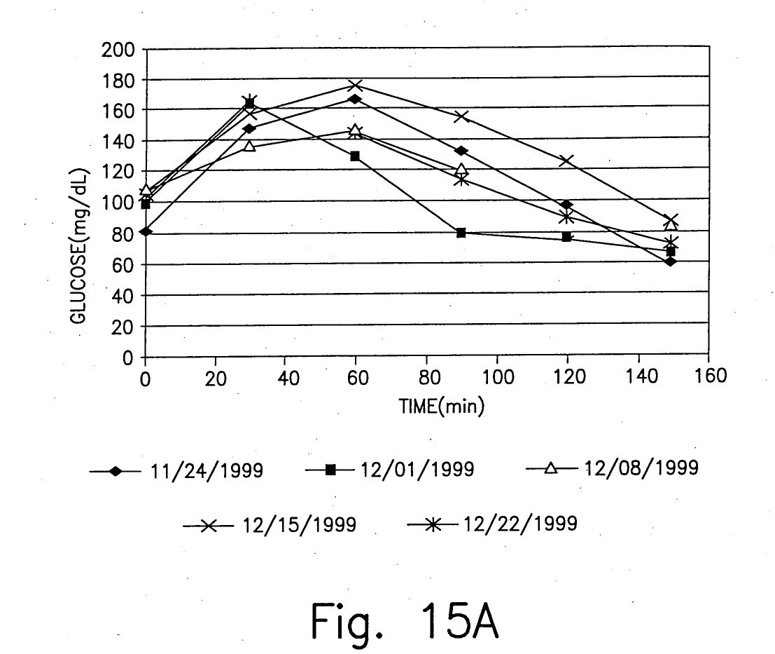

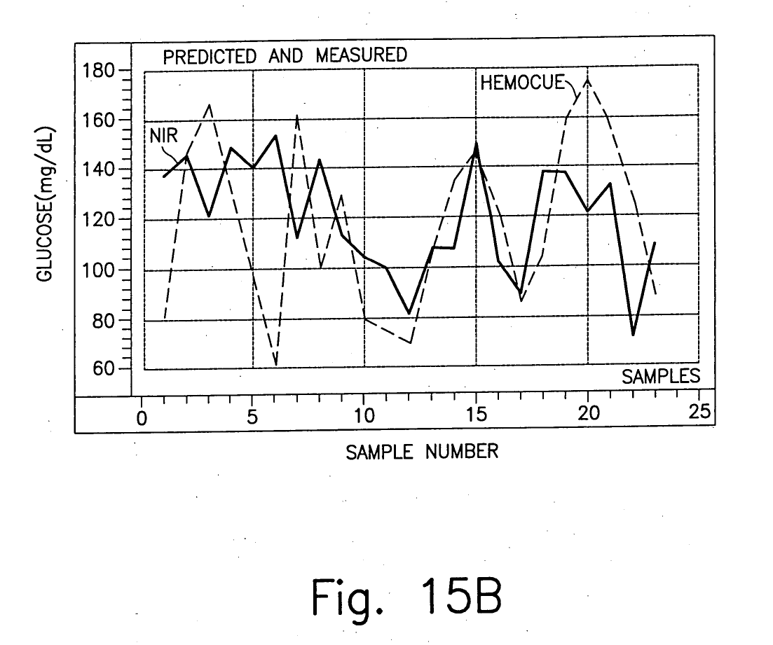

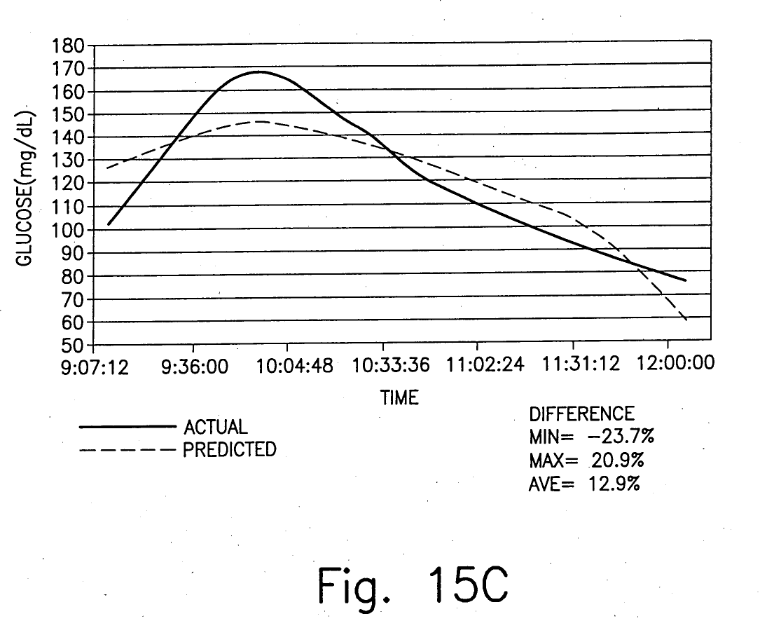

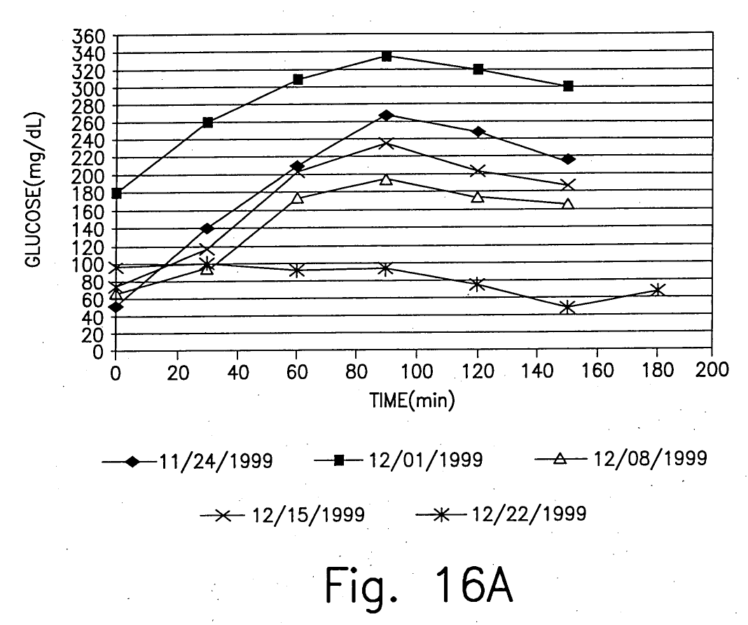

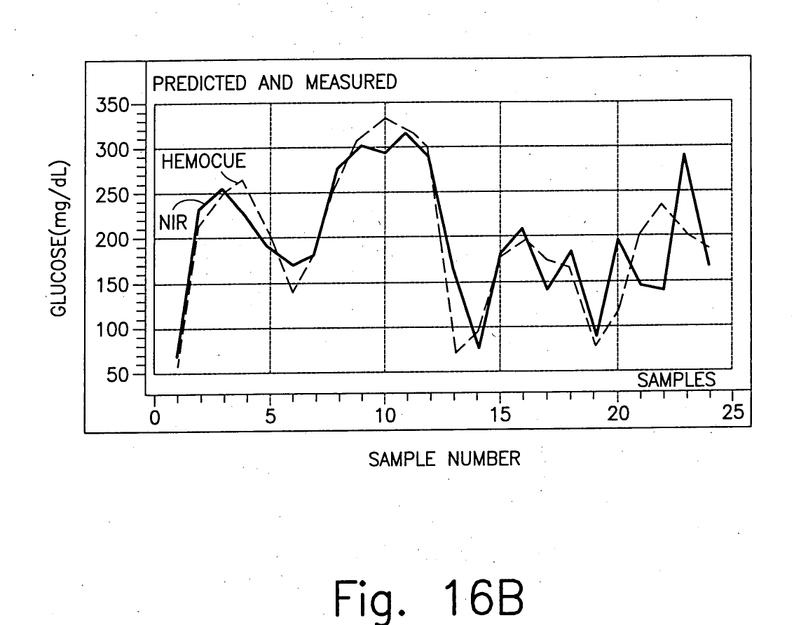

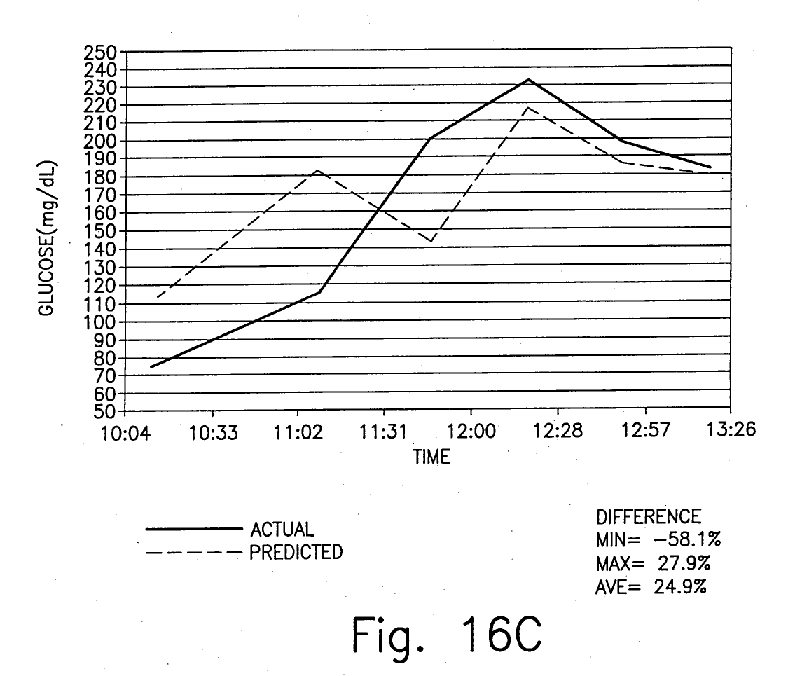

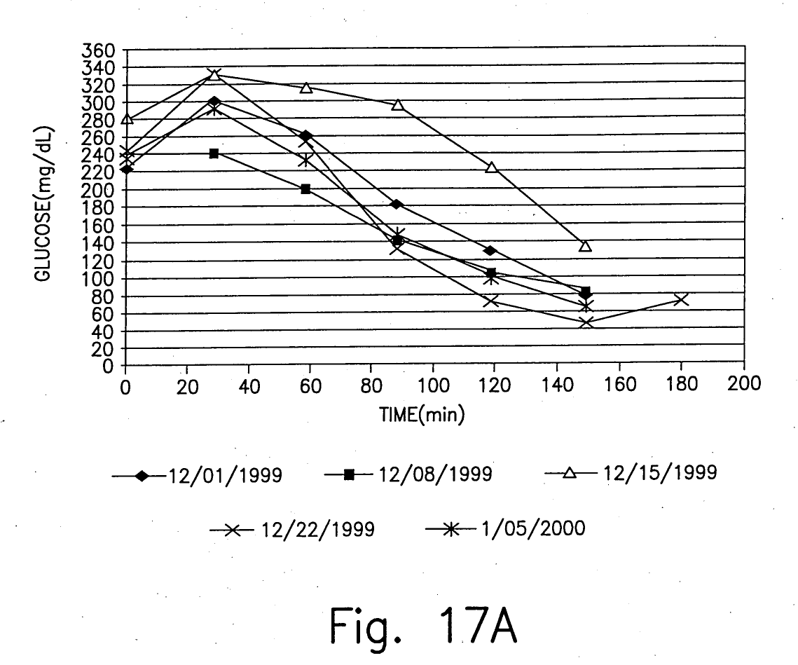

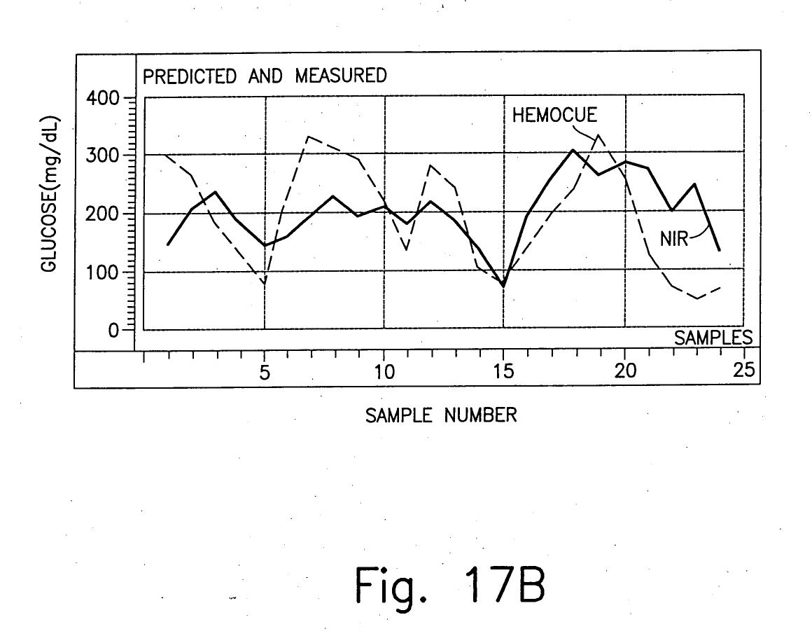

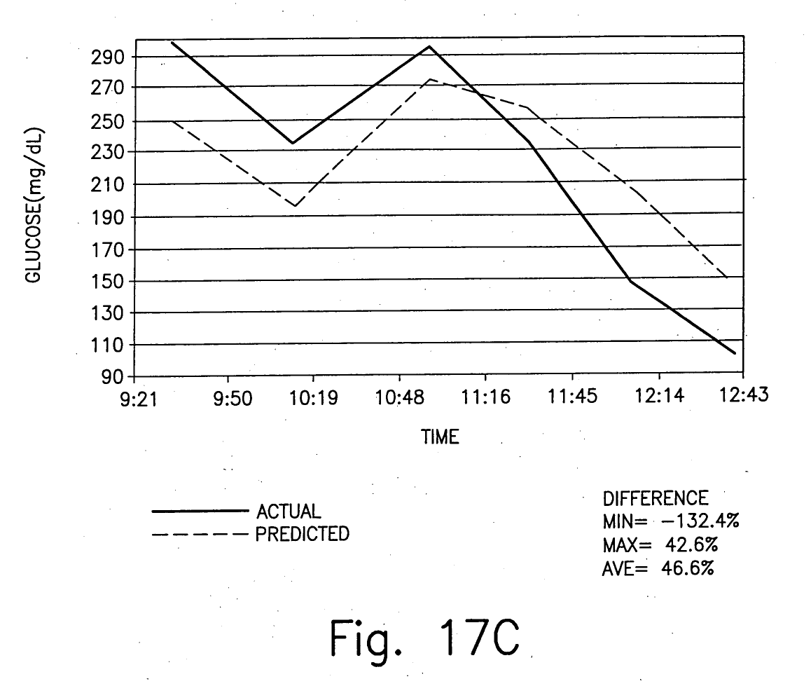

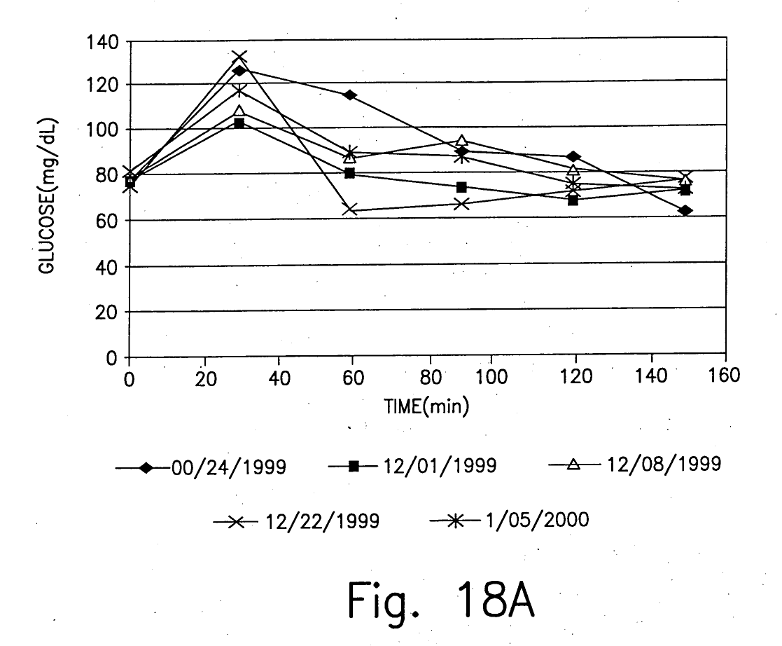

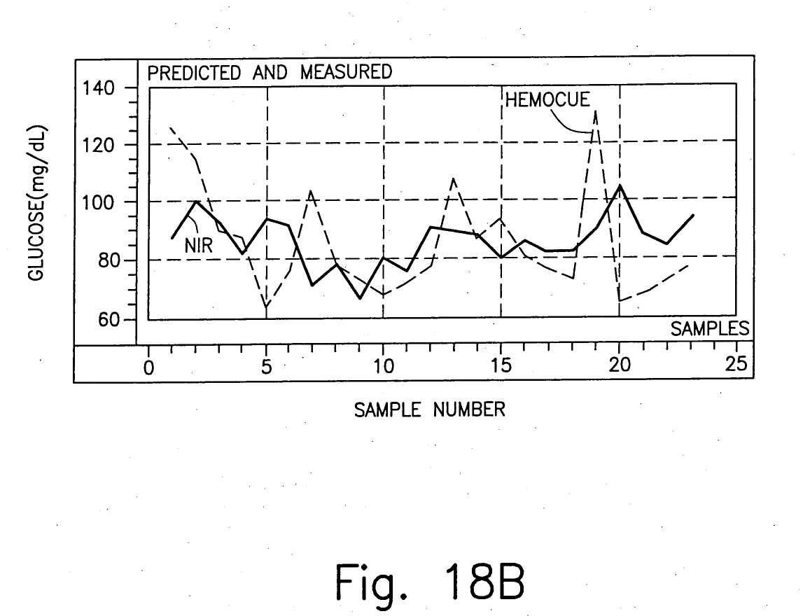

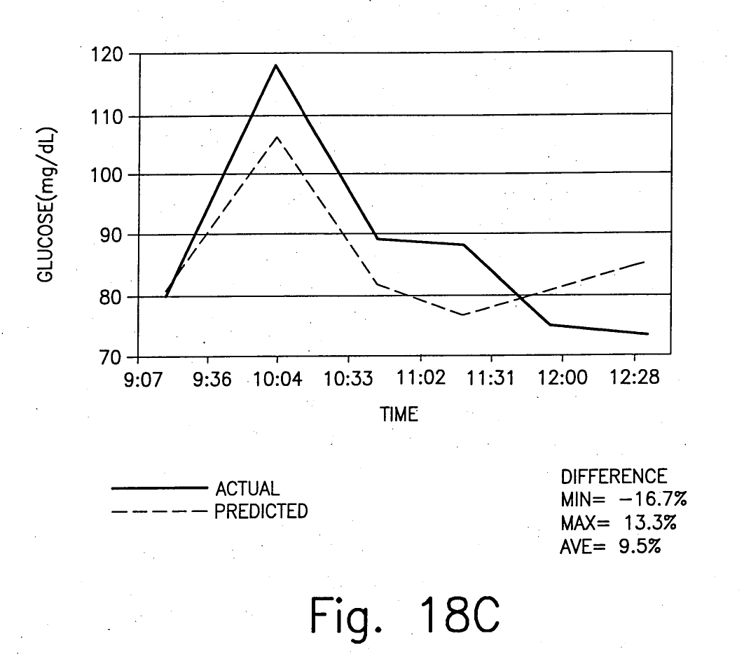

Table I summarizes the best modeling equation as calculated for each patient, based on the FOM, using the first four weeks of readings (unless otherwise indicated in the Patients Results Summary, infra). These modeling equations were then used to predict the fifth week readings. The results are summarized below with reference to FIGS. 13-18.

TABLE I

| | | | Spacing + | |
|---|---|---|---|---|
| Patient | Wavelength (nm) | Transformation Applied | Parameters Used | FOM |
| BDD | 1852, 2166, 2320, | Multiplicative scatter and 1^{st} | spacing 1 | 0.91 |
| | 2380, 2418 | Derivative | | |
| MLM | 1482, 1550, 1934, | 2^{nd} Derivative and Normalization | | 0.57 |
| | 2184, 2284 | | | |
| OMF | 1248, 1268, 1292, | Null and Ratio (1^{st} Derivative/1^{st} | ratio spacing 6 | 0.91 |
| | 2138, 2358 | Derivative) | | |
| PFK | 1384, 1496, 2058, | Null and Ratio (2^{nd} Derivative/ | ratio coeffcient: | 0.82 |
| | 2236, 2384 | 2^{nd} Derivative) | 42, 5, 0, −3, −4, | |
| | | | −3, 0 and 5 | |
| SLH | 1187, 1287, 1403, | Smoothing and 2^{nd} Derivative | Smoothing factor 8 | 0.87 |
| | 1519, 1775 | | Spacing 2 | |
| SLR | 1292, 1362, 1388, | Null and Ratio (2^{nd} Derivative/ | ratio spacing 12 | 0.87 |
| | 1830, 1894 | 2^{nd} Derivative) | | |
| TFD | 1268, 1856, 1882, | Null and Ratio (2^{nd} Derivative/ | ratio spacing 12 | 0.70 |
| | 2298, 2340 | 2^{nd} Derivative) | | |

Patient Results Summary

The patient begins each day in a fasting mode, eats breakfast, then has blood glucose measurements taken at ½ hour increments for 2½ hours.

Examples of patients' blood glucose data are shown in FIGS. 13A-C (elderly female african american diabetic OMF), 14A-C (middle aged female diabetic MLM), 15A-C (middle aged female diabetic SLH), 16A-C (middle aged african american male diabetic TFD), 17A-C (young male Caucasian diabetic BDD), and 18A-C (young female Caucasian non-diabetic PFK). In this graphic, FIGS. 13A, 14A, 15A, 16A, 17A, and 18A show the blood glucose profile of the patients OMF, MLM, SLH, TFD, BDD, and PFK, respectively. Multiple Linear Regression equations were generated for the patients and the NIR results calculated. A comparison of the NIR calculated values (dashed line) versus the Hemo-Cue® reference values (solid line) are shown for the patients OMF, MLM, SLH, TFD, BDD, and PFK in FIGS. 13B, 14B, 15B, 16B, 17B, and 18B, respectively. The values for the fifth week were calculated using an equation built from the first four weeks' data as an external validation test. That is, the spectral scans taken on the fifth week were analyzed by the modeling equation, and glucose values were generated. These values (dashed line) are compared with the reference (Hemo-Cue®) values (solid line) in FIGS. 13C, 14C, 15C, 16C, 17C, and 18C for the patients OMF, MLM, SLH, TFD, BDD, and PFK, respectively.

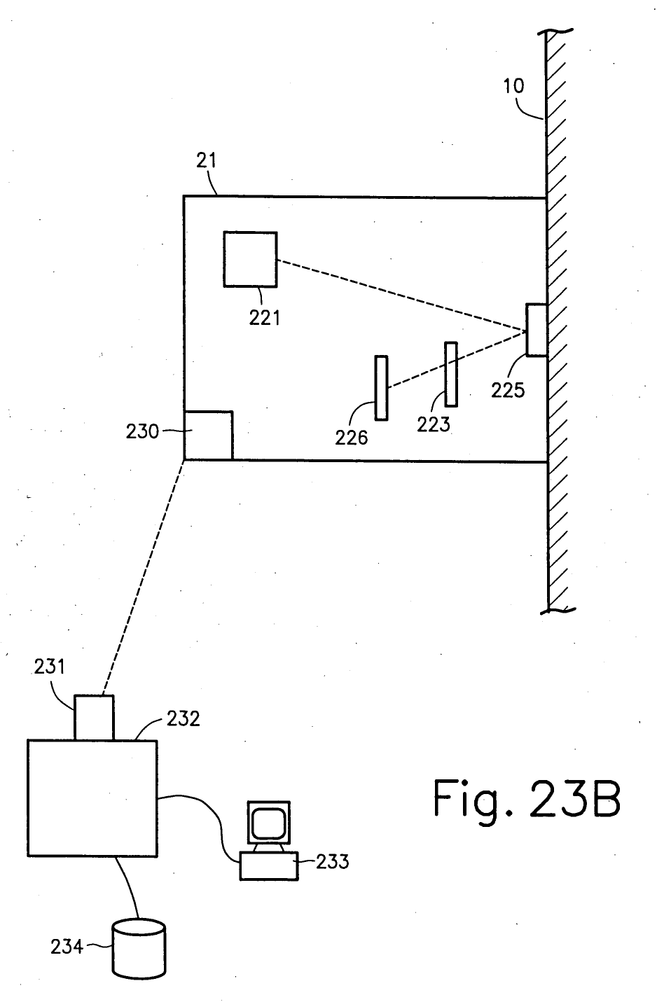

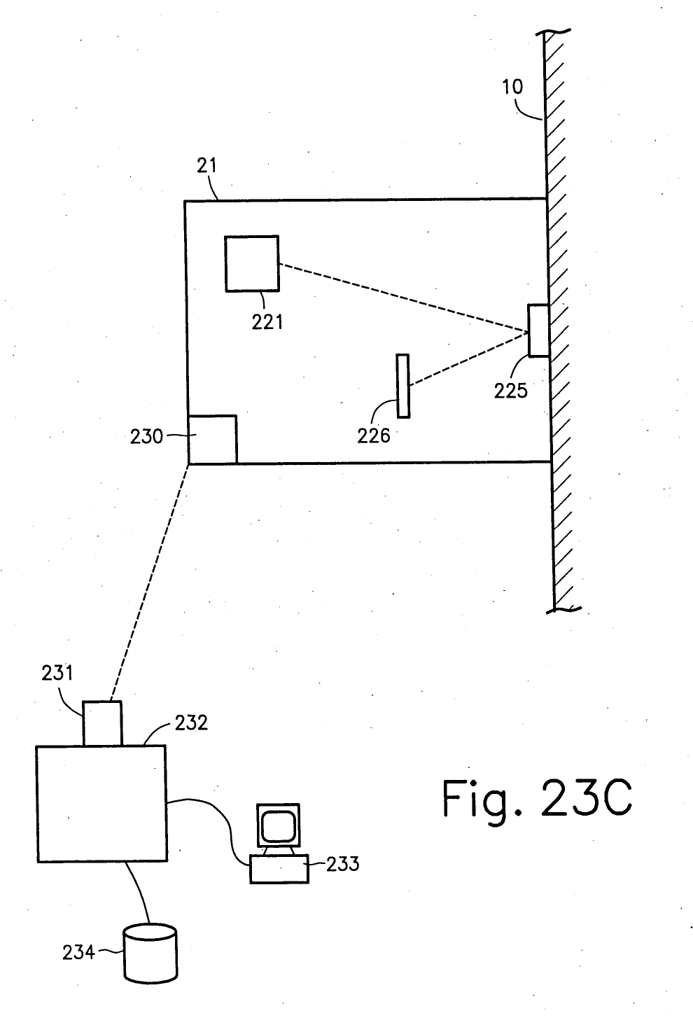

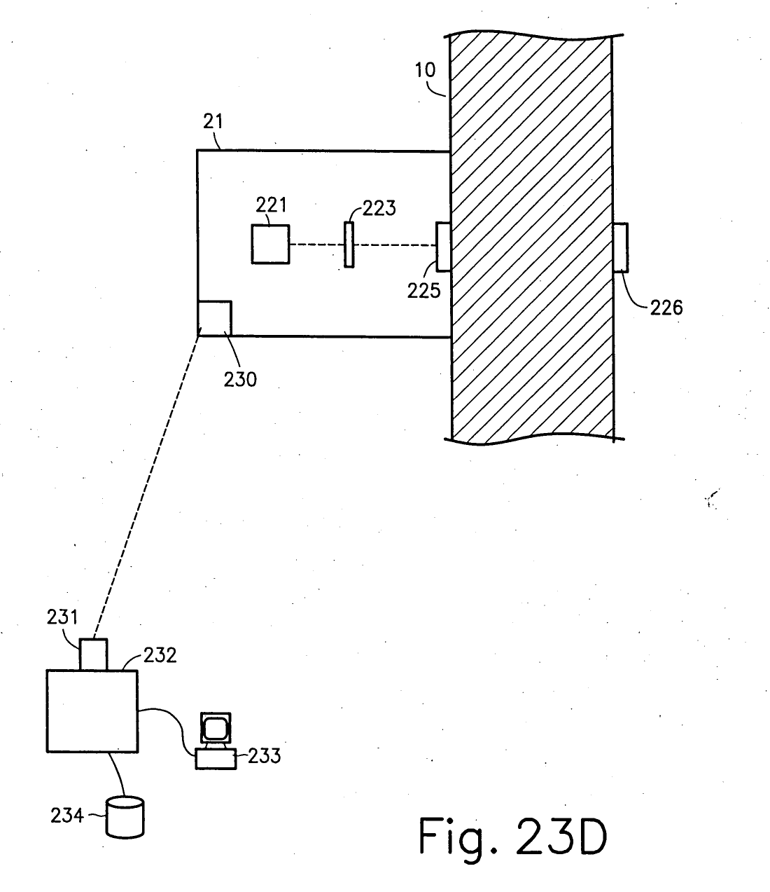

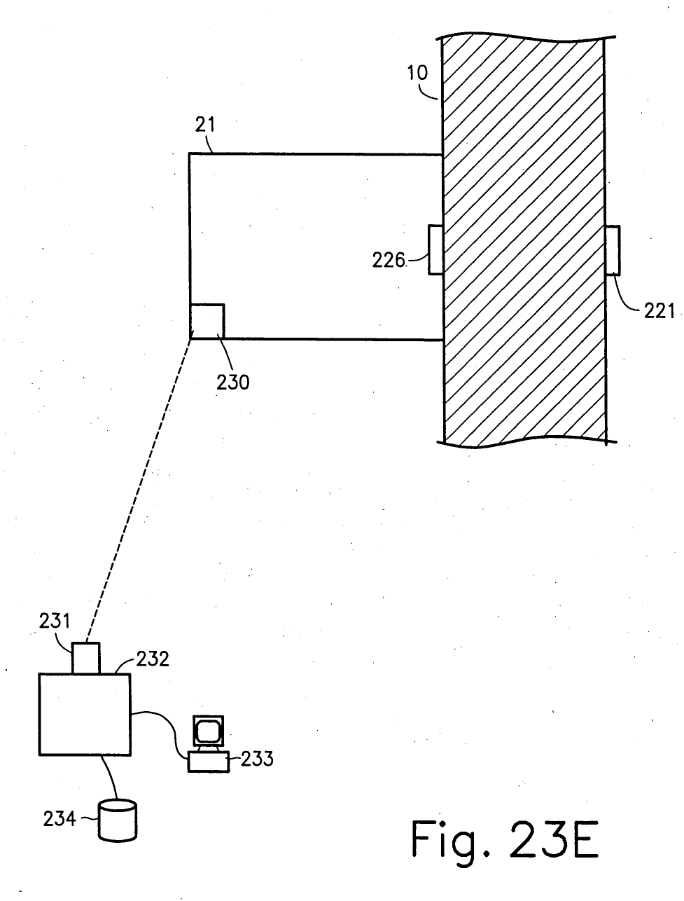

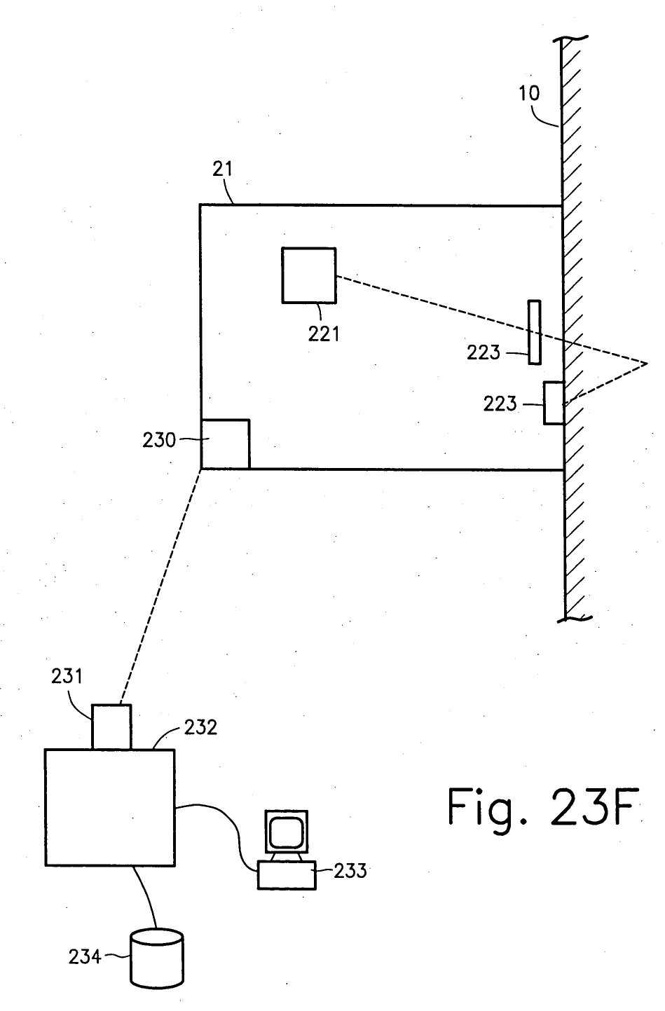

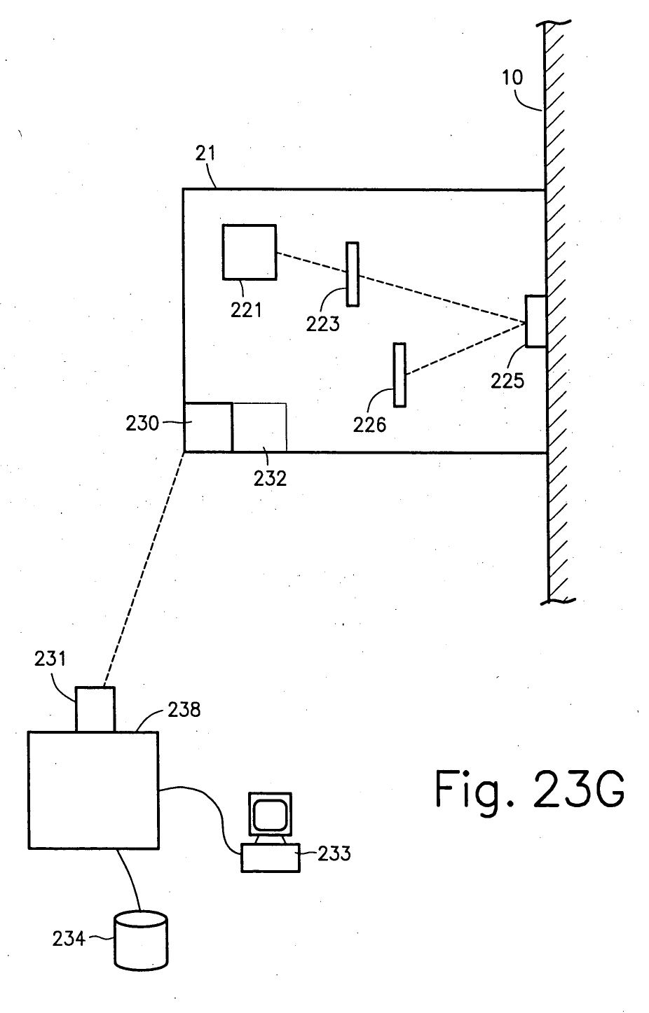

FIG. 23A shows a detailed schematic view of a first embodiment of remote spectrometer 21 adjacent body part 10 for generating a spectral scan of the body part. Body part 10 may be any body member or area suitable for taking a spectral scan, such as the palm of a hand, a finger, or the bottom of a foot, for example. As discussed above, a variety of different types of spectrometers are known in the art, such as grating spectrometers, FT (Fourier transformation) spectrometers, Hadamard transformation spectrometers, AOTF (Acousto Optic Tunable Filter) spectrometers, diode array spectrometers, filter-type spectrometers, scanning dispersive spectrometers, nondispersive spectrometers, and others as discussed below, and any of these may be used according to the present invention.

Spectrometer 21 in FIG. 23A has a light source 221, a light filtering device 223, a transparent element 225 and a detector 226. All or part of spectrometer 21 may be included in a hand-held device, such as a wand-like device, for example. In other embodiments, spectrometer 21 may be included in a table-top unit. Light source 221 generates a beam of light or radiation that passes through light filtering device 223. Light filtering device 223 separates the beam of polychromatic light into a monochromatic beam (or a beam having a narrower band of wavelengths than the polychromatic beam that is generated by light source 221 has), which then passes through a transparent element 225, such as a lens, that is adjacent to body part 10, as illustrated in FIG. 23A. In an embodiment of the present invention where spectrometer 21 is included in a wand-like device, transparent element 225 may be brought adjacent to body part 10 my moving the wand-like device to the body part. In an embodiment of the present invention where spectrometer 21 is included in a table-top unit, body part 10 may be moved into a position adjacent to transparent element 225.

After passing through transparent element 225, the beam of light or radiation impinges on body part 10. The reflected light is then absorbed by detector 226, which converts the beam of radiation into a digital signal. In an embodiment of the present invention utilizing an ATR spectrometer, the transparent element 225 may be the IRE and the beam could reflect off the interface between body part 10 and spectrometer transparent element 225 (e.g., where the body part and transparent element 225 contact one another). This configuration of the embodiment of FIG. 23A is “pre-dispersive” because the light generated by light source 221 passes through light filtering device 223 and is filtered to a monochromatic beam prior to it being dispersed by or reflected off body part 10.

The ATR crystal may be composed of ZnSe, Ge, SeAs, Cds, CdTe, CsI, C, InSb, Si, Sapphire (Al₂O₃), Anneled Glass, borosilicate crown glass, BK7 Anneled Glass, UBK7 Annealed Glass, LaSF N9 Anneled Glass, BaK1 Annealed Glass, SF11 Annealed Glass, SK11 Annealed Glass, SF5 Annealed Glass, Flint Glass, F2 Glass, Optical Crown Glass, Low-Expansion Borosilicate Glass(LEBG), Pyrex, Synthetic Fused Silica (amorphous silicon dioxide), Optical Quality Synthetic Fused Silica, UV Grade Synthetic Fused Silica, ZERODUR, AgBr, AgCl, KRS-5 (a TlBr and TlCl compound), KRS-6 (a TlBr and TlCl compound), ZnS, ZrO₂, AMTIR, barium fluoride, or diamond. Glass is transparent up to about 2200 nm, sapphire is transparent up to about 5 microns, and barium fluoride is transparent up to about 10 microns.

The entire ATR crystal or a portion thereof can be coated with a metallic coating, dielectric coating, bare aluminum, protected aluminum, enhanced aluminum, UV-enhanced aluminum, internal silver, protected silver, bare gold, protected gold, MAXBRIte, Extended MAXBRIte, Diode Laser MAXBRIte, UV MAXBRIte, or Laser Line MAX-R. The coating increases the amount of light reflected, thus, improving the accuracy of the data. Furthermore, the coating can be a material that only reflects a specific wavelength of light.

The ATR crystal can have a variety of shapes including, but not limited to, trapezoidal, cylindrical (e.g., pen shaped), hemispherical, spherical, and rectangular. Spherical ATR crystals reduce the beam diameter by a factor of two, thus, concentrating the beam to a smaller spot size.

The ATR crystal can be configured so that a beam of light enters the crystal, reflects off the interface, and exits the crystal. Such a crystal is known as a single bounce crystal. A single bounce crystal reduces Fresnel reflection losses due to the shorter path length of the beam. Because of the reduction of Fresnel reflection losses, the single bounce ATR may improve both qualitative and quantitative analysis. Multiple bounce ATR crystals can also be used. These provide the advantage of attenuating the beam multiple times, thus, providing a higher sensitivity to smaller concentrations.

It may be helpful, though not absolutely necessary, to place pressure on an IRE (e.g., the ATR crystal) to improve performance by increasing the amount of the substance (e.g., body part 10) that is in contact with the IRE. Pressure may be generated by the patient physically applying pressure to body part 10. Alternatively, the IRE may be mounted on a piston device that presses into body part 10 when in a forward position so that the spectrometer only scans when in this forward position.

In certain embodiments, detector 226 can be a photographic plate, a photoemissive detector, an imaging tube, a solid-state detector or any other suitable detector. Solid state detectors are preferred because of their small size. Possible detectors include, but are not limited to, silicon detectors (PDA, CCD detectors, individual photo diodes), photomultiplier tubes, Ga detectors, InSb detectors, GaAs detectors, Ge detectors, PbS detectors, PbSi photoconductive photon detectors, PbSe photon detectors, InAs photon detectors, InGaAs photon detectors, photoconductive photon detectors, photovoltaic photon detectors, InSb photon detectors, photodiodes, photoconductive cells, CdS photoconductive cells, opto-semiconductors, or HgCdTe photoconductive detectors. A single detector or an array of detectors can be used. The detector may connect to a processing unit, which can convert an interferogram signal to a spectrum.

Light filtering device 223 can be a prism, a grating filter (which is an optical device with a surface ruled with equidistant and parallel lines for the purpose of filtering light), an interferometer, or any other suitable filter. In an FTIR embodiment, a beam splitter and a movable mirror can be incorporated into spectrometer 21.

In this embodiment, as illustrated in FIG. 23A, spectrometer 21 is in wireless communication with a processing device 232 such that spectrometer 21 is capable of wirelessly transmitting spectral data to processing device 232 at a location separated from spectrometer 21, for example, a remote central location. In one embodiment, detector 226 converts the reflected beam into a digital signal that is then wirelessly transmitted to processor 232, where the reflected beam is analyzed. The digital signal generated by detector 226 of spectrometer 21 is first fed into a transmitter 230 located in or attached to spectrometer 21 and coupled to detector 226. Transmitter 230 then transmits the digital signal wirelessly to a receiver 231, which receives the digital signals on behalf of processing device 232. The digital signal can be transmitted from transmitter 230 to receiver 231 by any known technique in the wireless transmission art, as will be discussed in greater detail below.

In wireless transmissions of data, i.e., when the transmission of data does not use a physical connection (such as copper cable or fiber optics), electromagnetic radiation is useful to transmit information over long distances without damaging the information due to noise and interference. Various techniques for digital transmission of data are known in the art. Typically, the desired information is encoded into a digital signal and then may be modulated onto a carrier wave and made part of a larger signal. The signal is then sent into a multiple-access transmission channel, and electromagnetic radiation, e.g., radio, infrared, and visible light, is used to send the signal. After transmission, the above process is reversed at the receiving end, and the information is extracted. Examples of wireless data transmission via visible or NIR optical link include remote controls for televison and wireless data ports of laptop computers and personal digital assistants (PDAs). Examples of wireless data transmission via radio waves include cellular phones, wireless LAN and microwave transmission.

FIG. 23B illustrates a schematic representation of an embodiment of the present invention having a post-dispersive configuration. In this embodiment, the beam of light generated by light source 221 first impinges upon body part 10 and only then passes through light filtering device 223. After passing through light filtering device 223, the reflected light is absorbed by detector 226. This configuration is “post-dispersive” because the light generated by light source 221 passes through light filtering device 223 and is filtered to a monochromatic beam (or a beam having a narrower band of wavelengths than the polychromatic beam that is generated by light source 221 has) after is has been dispersed by or reflected off body part 10.

FIG. 23C illustrates a schematic representation of an embodiment of the present invention having a configuration in which spectrometer 21 does not comprise a light filtering device 223 at all. In this embodiment, because light filtering device 223 is not present, light generated by light source 221 is not passed through a filtering device either prior to being reflected off body part 10 or after being reflected off body part 10. Instead, light source 221 itself generates a beam of monochromatic light. Light source 221 can thus be, for example, a monochromatic laser.

FIG. 23D illustrates a schematic representation of an embodiment of the present invention in which light source 221 and detector 226 of spectrometer 21 are configured for a transmittance measurement. Light source 221 generates a beam of light, which passes through light filtering device 223 and onto body part 10. Transparent element 225 can also be included within this configuration, in order to focus or direct light onto body part 10. The beam of light then impinges detector 226, where the spectral data is measured. Alternatively, filtering device 223 could be situated adjacent to detector 226 (not shown), rather than adjacent light source 221, so that filtering of the light beam is performed post-dispersively, rather than pre-dispersively, as shown in FIG. 23D. In this embodiment, detector 226 may communicate with transmitter 230 or processing device 232 by a physical connection (e.g., a copper wire) or wirelessly, as discussed below.

FIG. 23E shows an embodiment of the present invention in a variation of FIG. 23D wherein the positions of light source 221 and detector 226 are effectively reversed. In this embodiment, light source 221 is still situated on the opposite side of body part 10 from detector 226 in order to facilitate transmittance spectrometry.

FIG. 23F shows a schematic representation of an embodiment of the present invention in a side view in which light source 221 and detector 226 are configured for a reflectance measurement. Light source 221 generates a beam of light, which passes through light filtering device 223 and onto body part 10. A portion of the beam of light reflected off the body part 10 continues onto detector 226, where the spectral data is measured.

FIG. 23G illustrates another embodiment of the present invention in a mode wherein processing device 232 is physically connected to spectrometer 21, rather than being remotely separated therefrom, as shown in FIGS. 23A-23F. In this embodiment, detector 226 converts the reflected beam into a digital signal that is then transmitted to processor 232 that is physically within, attached to or adjacent to spectrometer 21, where the reflected beam is analyzed. The connection between processing device 232 and detector 226 can be by conventional cables, wires or data buses, in which case transmission takes place through such physical connections. In this embodiment, there is no need for the digital signal generated by detector 226 to be fed into a transmitter located in or attached to spectrometer 21 and then transmitted wirelessly to a receiver on behalf of processing device 232.

However, a transmitter 230 may still be present and located in or attached to spectrometer 21 and coupled to processor 232. The digital signal that is analyzed and/or transformed by processing device 232 can be then fed to transmitter 230 for transmission to receiver 231 via a wireless connection. Transmitter 230 transmits the digital signal of data processed by processing device 232 wirelessly to receiver 231, which receives the digital signals on behalf of a remotely located device 238 for further processing. Device 238 may be a central processing device. As before, the digital signal can be transmitted from transmitter 230 to receiver 231 by any known technique in the wireless transmission art, as will be discussed in greater detail below. Processing device 232 may compress the digital signal so that it can be transmitted more efficiently or may modify the digital signal to facilitate error correction/detection, such as by inserting hamming code bits or error checking bits into the digital signal. The receiver can be physical connected to other devices (e.g., another processing device or display device).

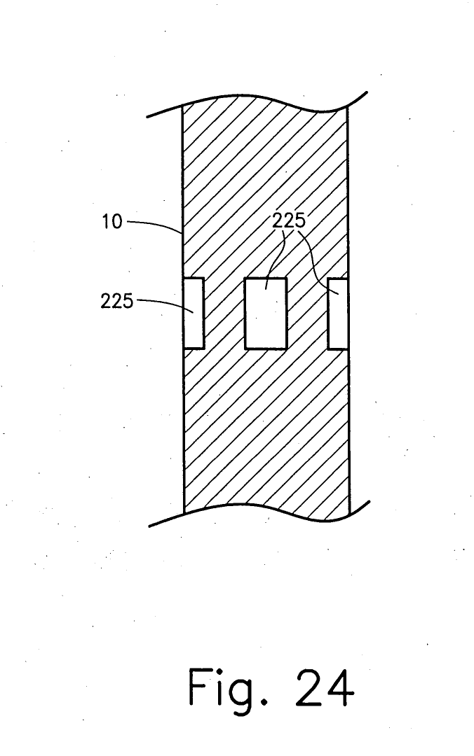

FIG. 24 shows an embodiment of the present invention wherein a plurality of transparent elements 225 are disposed about body part 10. In this embodiment, each transparent element 225 can be optically connected to a separate spectrometer 21. Thus, spectroscopic scans at different positions or angles about body part 10 can be taken. In this embodiment, each of the plurality of spectrometers 21 situated about body part 10 can be any of the embodiments discussed above, and as shown in FIGS. 23A-23G, or as discussed below. Thus, the various spectrometers can derive data regarding body part 10 through may variations and embodiments, so as to obtain readings that are verifiably accurate though the various techniques.

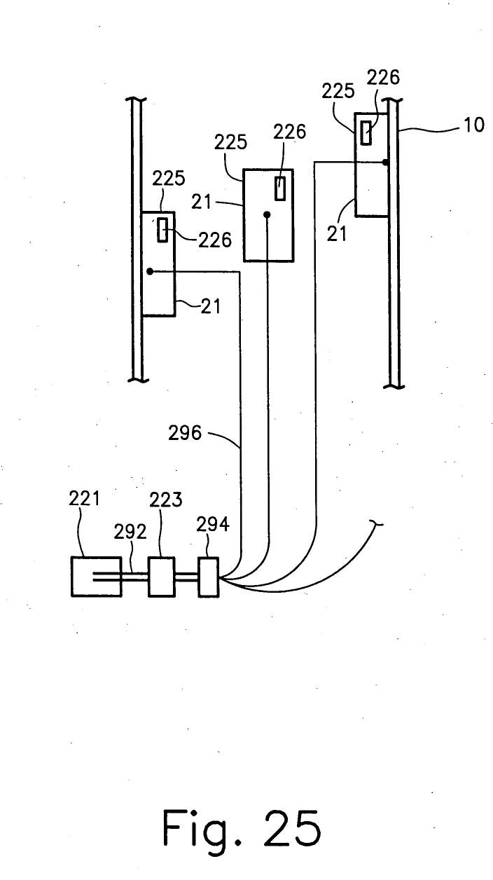

With further reference to FIG. 24 and FIGS. 23A and 23B, in an embodiment of the present invention, plurality of spectrometers 21 may be located in a region of body part 10, so that light sources 221 flood the region with large amounts of light. A “ring of light” may thus be provided. Large amounts of light provide a relatively large signal-to-noise ratio for spectral analysis purposes. Light sources 221 could be NIR light emitting diodes (LEDs), for example, since such devices generate relatively little heat. Detectors 226 for each spectrometer 21 may be diode arrays or linear variable filter detectors (such as the MicroPac family of products available from OCLI), for example. Alternatively, detectors 226 could each include a number of individual diodes having a respective filter 223 for excluding all but a desired wavelength of light, as in the embodiment shown in FIG. 1B. In this way, intensity values at different wavelengths may be measured for each position on body part 10. In other embodiments of the present invention, a fiber optic bundle split into individual optical fibers, as shown in FIG. 25 below, could be used as the light source for flooding the desired region with light.

FIG. 25 shows another embodiment of the invention wherein a plurality of spectrometers 21 or transparent elements 225 are disposed about the circumference of body part 10. In this embodiment, light source 221 includes fiber optic bundle 292 optically connected to filtering, or monochromator, device 223. Filtering device 223 may be a grating, interferometer, filter wheel, or other suitable device for producing a monochromatic beam of light in each fiber of fiber optic bundle 292. Splitter device 294 is provided for splitting fiber optic bundle 292 into a plurality of individual fibers 296, which illuminate respective multiple positions, or angles, on body part 10 via respective transparent elements 225. Components 221, 292, 223, and 294 may be housed in a common housing which may be hand-held and may be secured to body part 10 or another body part. Respective detectors 226 are provided at each position or angle body part 10 for detecting light diffusively reflected, transmitted, etc., from the body part. Any desired number of spectrometers 21, and hence, of illumination and detection (sampling) positions on body part 10, may be provided situated in a desired configuration about the circumference of the body part. Moreover, the spectrometers may be positioned at different longitudinal levels on body part 10, as shown in FIG. 25.

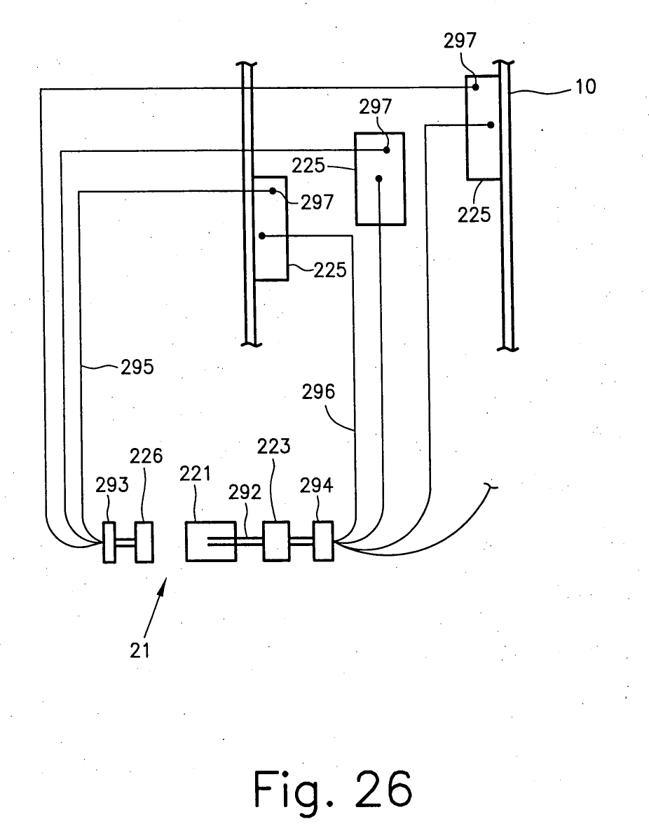

FIG. 26 shows an embodiment of the invention having a single spectrometer 21 with a plurality of transparent elements 225 disposed as different longitudinal levels about the circumference of body part 10. In this embodiment, like that shown in FIG. 25 and discussed above, light source 221 including fiber optic bundle 292 is provided. Fiber optic bundle 292 is optically connected to filtering, or monochromator, device 223. Filtering device 223 may be a grating, interferometer, filter wheel, or other suitable device for producing a monochromatic beam of light in each fiber of fiber optic bundle 292. Splitter device 294 is provided for splitting fiber optic bundle 292 into plurality of individual fibers 296, which illuminate respective multiple positions, or angles, body part 10 via respective transparent elements 225. Components 221, 292, 223, and 294 may be housed in a common housing which may be hand-held and may be secured to body part 10 or another body part.

In the embodiment shown in FIG. 25, single detector 226 is provided. Detector 226 may be a photo diode array or a single element detector combined with a monochromator interferometer, for example. Switching device 293 interfaces detector 226 with fiber optic light guides 295, each connected to a respective sampling position 297 at a respective transparent element 225. Each fiber optic light guide 295 receives diffusively reflected or transmitted, etc., light from body part 11. Switching device 293 selects one sampling position 297 at a time and presents the received light to detector 226. This embodiment may be used to read out each sampling position 297 in a desired sequence in a relatively short period of time. Any desired number of sampling positions 297 may be provided situated in any desired configuration about body part 10. In other embodiments of the present invention (not shown) respective individual light sources 221 may be provided for each transparent element 225, instead of using splitter device 294 plurality of individual fibers 296.

As stated above, the digital signal can be transmitted from transmitter 230 to receiver 231 by any known technique in the wireless transmission art, such as transmission using carrier waves in the IR, radio, optical or microwave region of the wavelength spectrum. Infrared (IR) transmission uses an invisible portion of the spectrum slightly below the visible range. The IR transmission can be directed, which requires a direct line-of-site, or diffuse, which does not require line of sight.

Radio transmission uses the radio region on the spectrum, which is located above the visible portion of the spectrum. Suitable devices that allow digital signals to be transmitted in the FM radio region of the spectrum are made by Aeolus and Xircon. In certain embodiments, Xircon's Core Engine can be directly embedded in the electronics of transmitter 230 and receiver 231. In certain embodiments, transmitter 230 and receiver 231 can be linked to a Wi-Fi certified wireless network anywhere in the world, and GSM/CDMA, LAN and WAN connections can also be provided, using devices provided, for example, by 3Com or Nokia.

The digital signal may also be wirelessly transmitted from transmitter 230 to receiver 231 in the microwave frequencies, which are located below the visible range of the spectrum. Nokia microwave radios, for example, can provide a microwave link between transmitter 230 and receiver 231.

Optical devices, such as those based on lasers, can also be used to transmit the digital signal from transmitter 230 to receiver 231.

Once receiver 231 receives the digital signal from transmitter 230, receiver 231, in turn, transmits the digital signal to a processing device 232 to which it is coupled, by any known method. Processing device 232 can be physically coupled to receiver 231, as illustrated in FIG. 23A such as through conventional cables, wires or data buses, in which case such transmission takes place through such physical connections. Processing device 232 can also be separate from receiver 231 and coupled thereto wirelessly, in which case such transmission from receiver 231 to processing device 232 takes place through any of the wireless methods discussed above. Upon receipt of the digital signal from receiver 231, processing device 232 can then process the digital signal as well as transmit the digital signal to peripherals, such as a display device 233 and/or storage device 234. In a network embodiment, processing device 232 can transmit the digital signal to subsequent processing devices. In the embodiment shown in FIG. 23G, for example, processing device 232 can transmit the signal to a further remotely located device 238, which can transmit the digital signal to peripherals, such as a display device 233 and/or storage device 234.

The communication between spectrometer 21, receiver 231 and the processing device 232 in FIGS. 23A-F (as well as with remote device 238 in FIG. 23G) can also be via a wireless peer-to-peer network. In such a network, spectrometer 21 and attached transmitter 230 send the digital signal to processing device 232 and receiver 231, which can, for example, be a laptop personal computer equipped with wireless adapter card, via a wireless connection. From processing device 232, a user can analyze the digital signal, transform the digital signal, compare the digital signal to the data set in storage device 234 or display the digital signal on display device 233. Processing device 232 can be moved, so that communication with other spectrometers is possible without the need for extensive reconfiguration. In this embodiment, spectrometer 21 and transmitter 230 function as a client, while processing device 232 acts as a server.

A data reduction technique, such as a partial least squares, a principal component regression, a neural net, a classical least squares (often abbreviated CLS, and sometimes called The K-matrix Algorithm), or a multiple linear regression analysis can then be used to generate a modeling equation from the digital signal.

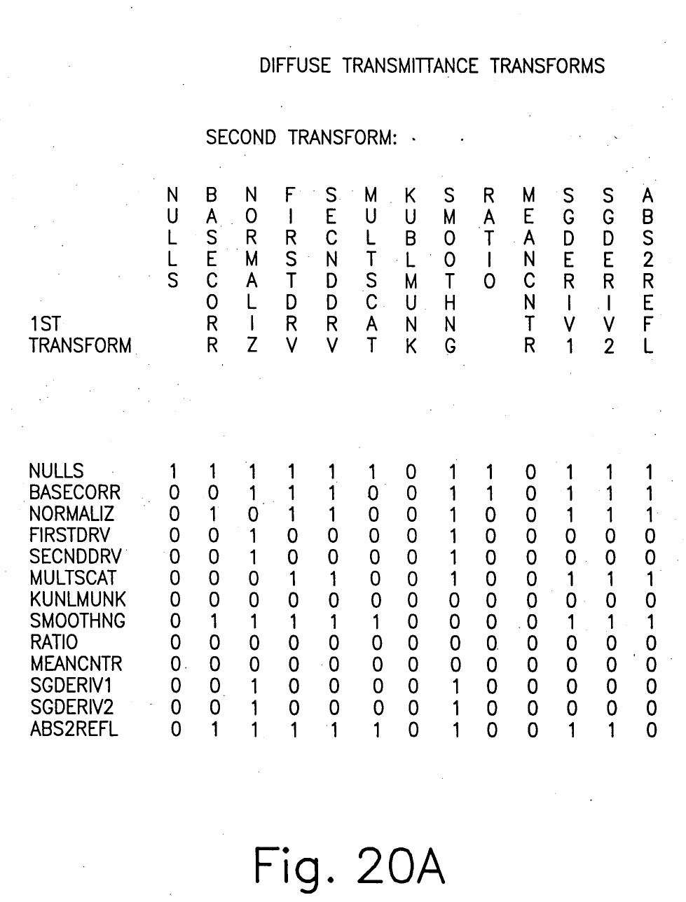

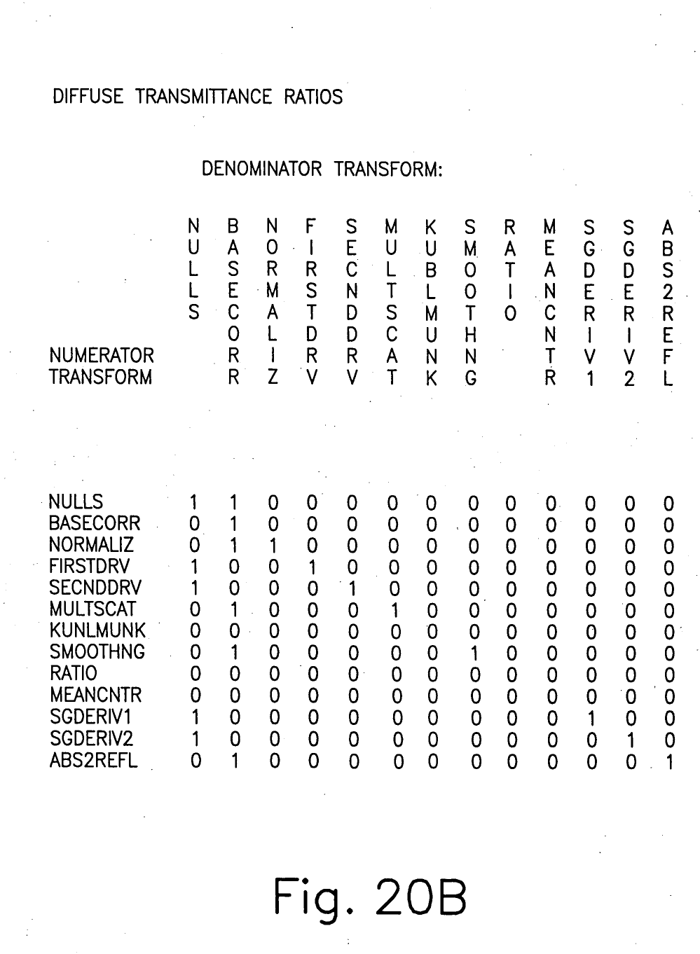

In certain embodiments, processing device 232 may regenerate and/or recalibrate the modeling equations using one or more techniques as discussed above with reference to FIGS. 19-22. A user may select which techniques to use in transforming or modeling the data. In certain embodiments, the techniques may also be selected pursuant to a set of rules specifying which algorithms to use for a particular type of composition.

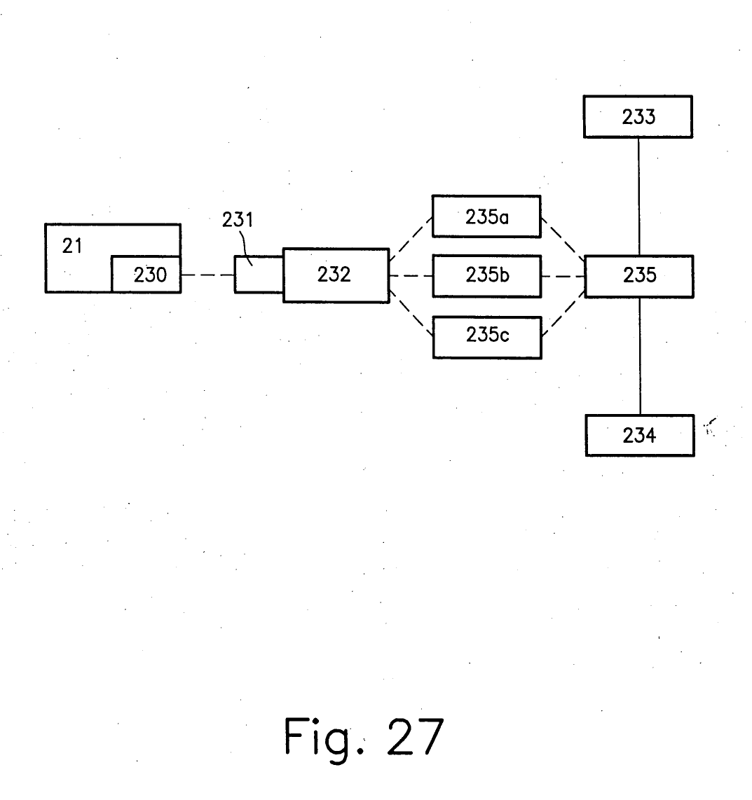

FIG. 27 shows a schematic representation of a configuration for transmitting the digital signal between remote spectrometer 21 and central processing device 236, with multiple processing devices 232 and 235a, 235b, 235c arranged in a distributive network. In this configuration, spectrometer 21 includes transmitter 230 and wirelessly transmits a digital signal to receiver 231. The first processing device 232 (e.g., a routing device) receives the digital signal from receiver 231 and transmits a first portion of the digital signal to processing device 235a (e.g., a computer in a distributive network), a second portion of the digital signal to processing device 235b, and a third portion of the digital signal to processing device 235c. Processing devices 235a, 235b, 235c perform various functions on their respective portions of the digital signal in parallel (e.g., transformations of the digital signal) and then each transmits a modified digital signal to a fifth processing device 236 (e.g., a personal computer). Processing device 236 analyzes and transmits the digital signal to display device 233 (e.g., a monitor) and to storage device 234 (e.g., a hard disk). The communication between any of the devices can be via wireless communication, or the devices can be physically connected (e.g., copper wire or fiber optic cable).

Although only one spectrometer 21 with a transmitter 230 is shown in FIG. 27, an arrangement with a plurality of spectrometers, each connected to the same processing unit or distributed over the plurality of processing units, is possible. Similarly, it should be understood that the present invention is not limited to the number or configuration of processing devices 232, 235a, 235b, 235c and 236 shown in FIG. 27. Other configurations, with more or fewer processing devices, are possible.

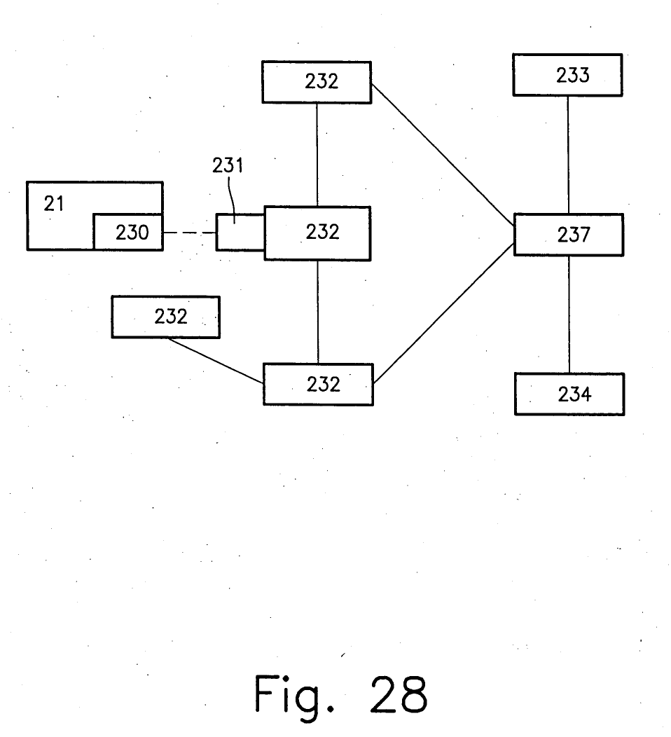

FIG. 28 shows a schematic representation of another configuration for transmitting the digital signal to a processor, between a plurality of processing devices 232 and a central processing device 237. Spectrometer 21 with associated transmitter 230 wirelessly transmits the digital signal to a receiver 231, which is integrated within or coupled to one of processing devices 232 and in communication therewith. Each processing device 232 (e.g., a routing device) transmits the digital signal either to central processing device 237 or to a different processing device 232. Central processing device 237 analyzes the digital signal. Central processing device 237 processes the digital signal and may also transmit the digital signal or selected portions of the data contained therein to display device 233 (e.g., a monitor) where it is displayed in human readable form. Central processing device 237 may also transmit the digital signal or selected portions therein to storage device 234 (e.g., a hard disk). The communication between any of the devices can be via wireless communication (e.g., radio Waves). The devices can also be physically connected (e.g., by wire or fiber optic cable). Furthermore, central processing unit 237 can be mobile, such as by being mounted in a mobile platform (e.g., a laptop or hand-held device) or by itself having a mobile structure, such as a lap-top computer, so that central processing unit 237 can be placed at different positions with respect to the network. Although only one spectrometer 21 with a transmitter 230 is shown in FIG. 28, an arrangement with a plurality of spectrometers 21, each connected to the same processing unit or distributed over the plurality of processing units 232, is possible.

In certain embodiments, transmitter 230 can be a transmitter/receiver device, so that the spectrometer 21 may function with a Global Positioning System (GPS). GPS technology allows tracking of the device and may prove helpful if the spectrometer is lost or stolen. Furthermore, the GPS coordinates of a home location of spectrometer 21 can be sent, along with the digital signal, to a central database, so that, if a problem is detected regarding spectrometer 21, a repair technician could be sent directly to the spectrometer by using the spectrometer's GPS coordinates.

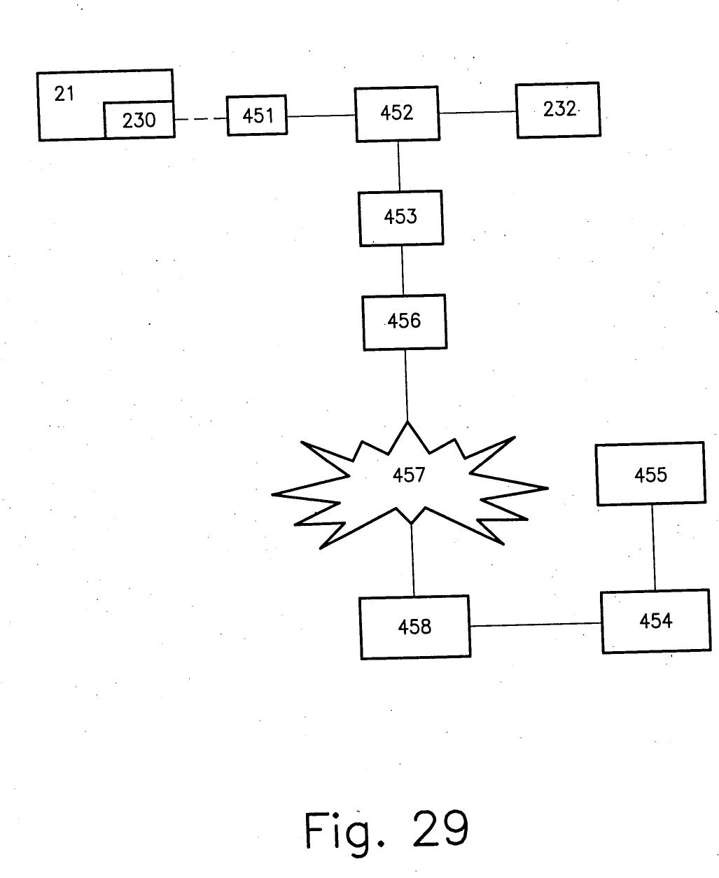

FIG. 29 shows a schematic representation of a networking arrangement for transmitting the digital signal in accordance with another embodiment of the present invention. The wireless access point 451 can be any suitable device, such as Linksys's WAP11. Spectrometer 21 wirelessly transmits the digital signal to wireless access point 451 by transmitter 230. Wireless access point 451 then transmits the digital signal to a router 452 via a physical connection. Router 452 can be any suitable device, such as a Linksys' BEFSR41 4-port cable/DSL router. Router 452, in turn, transmits the data to processing device 232 and a cable modem 453. Router 452 can be connected to processing device 232 and cable modem 453 by any suitable device, such as, for example, a 10BaseT connector. At processing device 232, a user may perform functions on the data, view the data and/or store the data. Cable modem 453 transmits the digital signal over existing phone lines to a communication provider 456, e.g., AT&T, which in turn uses existing networks to transfer the digital signal to the Internet 457. From the Internet 457, the digital signal is received by another communication provider 458, e.g., America Online, which transmits the digital signal to a second wireless access point 454. Second wireless access point 454 can be any suitable device, such as a Linksys' WAP11. Provider 458 can be connected to second wireless access 454 point by, for example, existing phone lines. Second wireless access point 454 transmits the digital signal to a mobile processing device 455, such as a laptop computer, equipped with a wireless card. The wireless card can be any suitable device, such as, for example, 3Com's Wireless AirConnect PC card. From mobile processing device 455 with the wireless card or the processing device 452, a user can perform functions on the digital signal, the digital signal can be displayed and/or the digital signal can be stored.

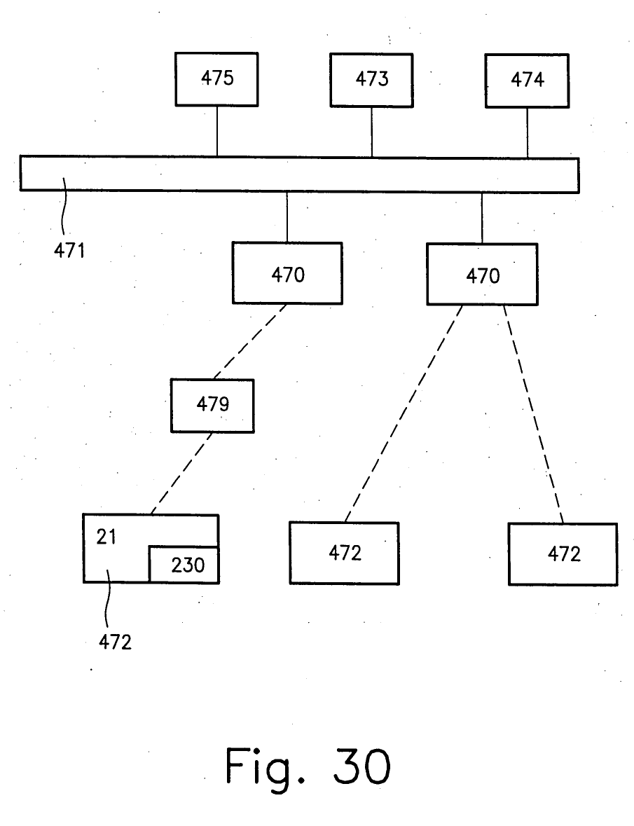

FIG. 30 illustrates a plurality of clients 472 and a plurality of access points 470 arranged in a wireless network. In this embodiment, spectrometer 21 and transmitter 230 function as one of the clients 472. Clients 472 can also be processing device 232 (e.g., a PC or a lap-top). Each client 472 can wirelessly transmit the digital signals to a wired network 471 by transmitting to one of access points 470. Access points 470 extend the range of the wired network 471, effectively doubling the range at which the devices can communicate. Each access point 470 can accommodate one or more clients 472, the specific number of which depends upon the number and nature of the transmissions involved. For example, a single access point 470 can be configured to provide service to fifteen to fifty clients 472. In certain embodiments, clients 472 may move seamlessly (i.e., roam) among a cluster of access points 470. In such an embodiment, access points 470 may hand client 472 off from one to another in a way that is invisible to the client 472, thereby ensuring unbroken connectivity.

Once the digital signal enters wired network 471, the digital signal can be relayed to a server 475, the display device 473 and the storage device 474, as well as to other clients 472. Server 475 or other clients 472 can convert the digital signal to a spectrograph and/or perform various algorithms on the digital signal.

In certain embodiments, an extension point 479 is provided. Extension points 479 augment the network of access points 470 and function like access points 470. However, extension points 479 are not tethered to wired network 471 as are access points 470. Instead extension points 479 communicate with one-another wirelessly, thereby extending the range of network 471 by relaying signals from a client 472 to an access point 470 or another extension point 479. Extension points 479 may be strung together in order to pass along messaging from an access point 470 to far-flung clients 472.

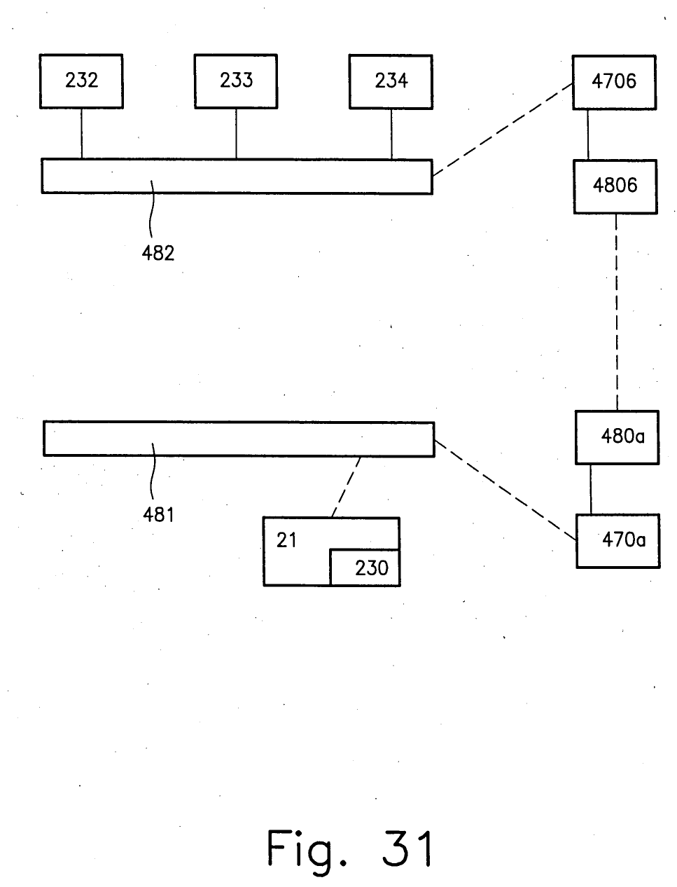

FIG. 31 shows a schematic representation of a networking arrangement for transmitting the digital signal in accordance with yet another embodiment of the present invention. Communication between first and second networks 481,482 is by directional antennas 480a,480b. Each antenna 480a,480b targets the other to allow communication between networks 481,482. First antenna 480a is connected to first network 481 via an access point 470a. Likewise, the second antenna 480b is connected to second network 482 by an access point 470b. The digital signal from spectrometer 21 is transmitted by transmitter 230 to first network 481 and is then transmitted to the directional antenna 480a by being relayed over the nodes of first network 481. The digital signal can then be transmitted to second directional antenna 480b on second network 482. Second network 482 then relays the digital signal to processing device 232, display device 233 and/or the storage device 234.

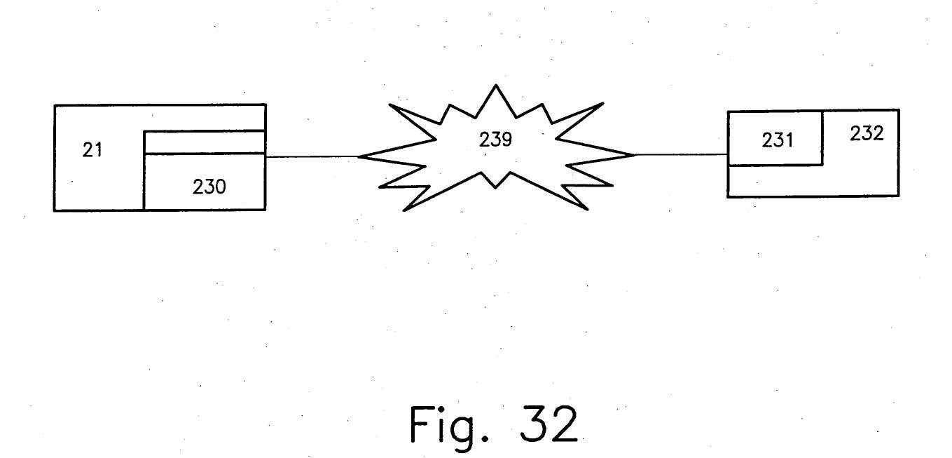

FIG. 32 shows the communication between spectrometer 21 and processing unit 232 via an existing wireless network 239. The data from spectrometer 21 is fed into a transmitter 230 located in or attached to spectrometer 21. Transmitter 230 can be, for example, the type of transmission device used in a conventional cell phone. Transmitter 230 then connects to the processing device 232 equipped with a receiver 231 (e.g., a receiver used in current cell phone technology) by opening a communication channel specific to the processing device 232 on wireless network 239 (e.g., dialing a cell phone number). Once the communication channel is established, the digital signal is then transferred to processing device 232 by routing the digital signal through the existing wireless network 239. Processing device 232 can then be connected to another network or a display device and/or storage device. Wireless network 239 can be any suitable network, such as, for example, SkyTel or Nokia's communication network. In certain embodiments, wireless network 239 can be included as part of a wireless LAN, wireless WAN, cellular/PCS network (e.g., by using a transceiver equipped with a CPDP modem), digital phone network, proprietary packet switched data network, One-way Pager, a Two-way Pager, satellite, Wireless local loop, Local Multi-point Distribution Service, Personal Area Network, and/or free space optical networks.

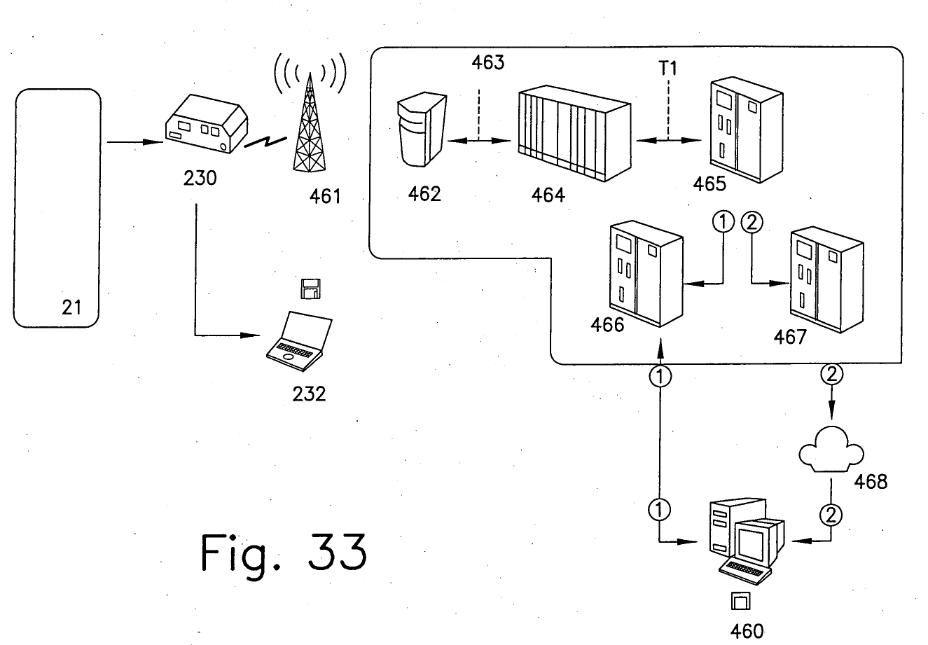

FIG. 33 shows the communication between the spectrometer 21 and an application server 460 via a wireless network. Spectrometer 21 sends the digital signal to transmitter 230, which can be, for example, Xircon's Redhawk II™. Transmitter 230 then wirelessly sends the digital signal to processing device 232, which can be, for example, a laptop computer, and to a long range transmission device 461, which transmits the digital signal to a base transceiver station 462 via a modulated radio wave. Then, through a T1 line 463, the digital signal is transmitted to a base station controller 464, which in turn transmits the digital signal to a mobile switching center 465. Based on a pre-defined user setting, mobile switching center 465 transmits the digital signal to either an interworking function device 466 or a short message center 467. If the digital signal is sent to interworking function device 466, interworking function device 466 then transmits the digital signal to an application server 460. However, if the digital signal is sent to short message center 467, short message center 467 routes the digital signal over the Internet 468 and on to the application server 460. Application server 460 provides for display of the digital signal, transfer of the digital signal to a client of server 460, analysis of the digital signal, and/or storage of the digital signal. Application server 460 can be any suitable device, such as, for example, an IBM compatible Gateway personal computer.

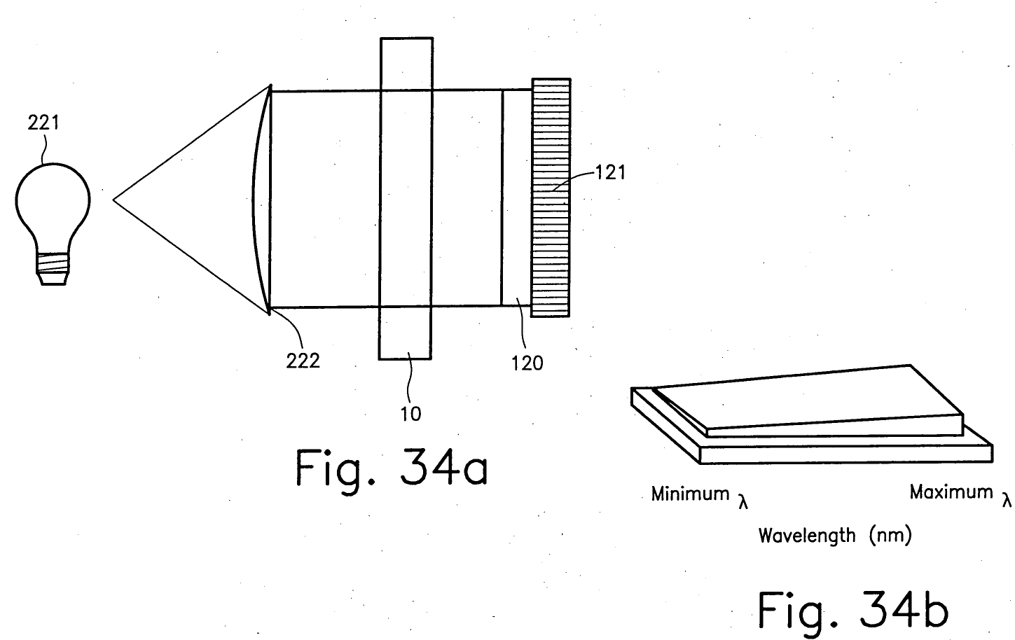

FIGS. 34A-B show an illustrative remote spectrometer for performing spectral scans. As illustrated in FIG. 34A, a multiple wavelength photometer has light source 221 that produces a light beam that is focused and directed onto body part 10 by focusing optics 222. The light that is transmitted through body part 10 is passed through a linear variable filter120 to an array detector 121 in order to filter and receive a number of specific, predetermined narrow bands of wavelengths simultaneously. Linear variable filters are well known in the art and are described in, for example, U.S. Pat. No. 6,057,925 to Anthon, U.S. Pat. No. 5,166,755 to Gat and U.S. Pat. No. 5,218,473 to Seddon et al., and are shown schematically in FIG. 34B. Focusing optics 222, linear variable filter120 and array detector 121 may be used and positioned very much in the same way as filter 223 and detector 226 are used and positioned in the embodiments and versions discussed elsewhere herein, such as those shown in FIGS. 23A-G.

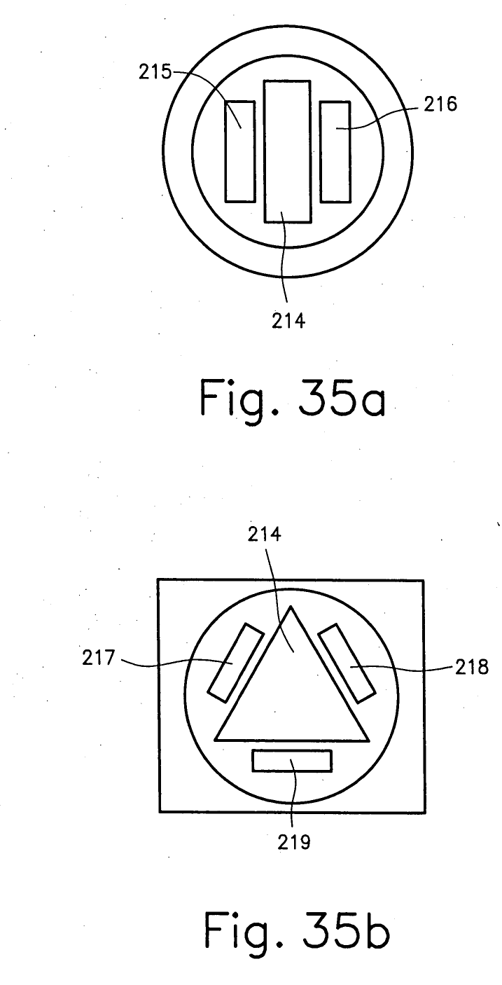

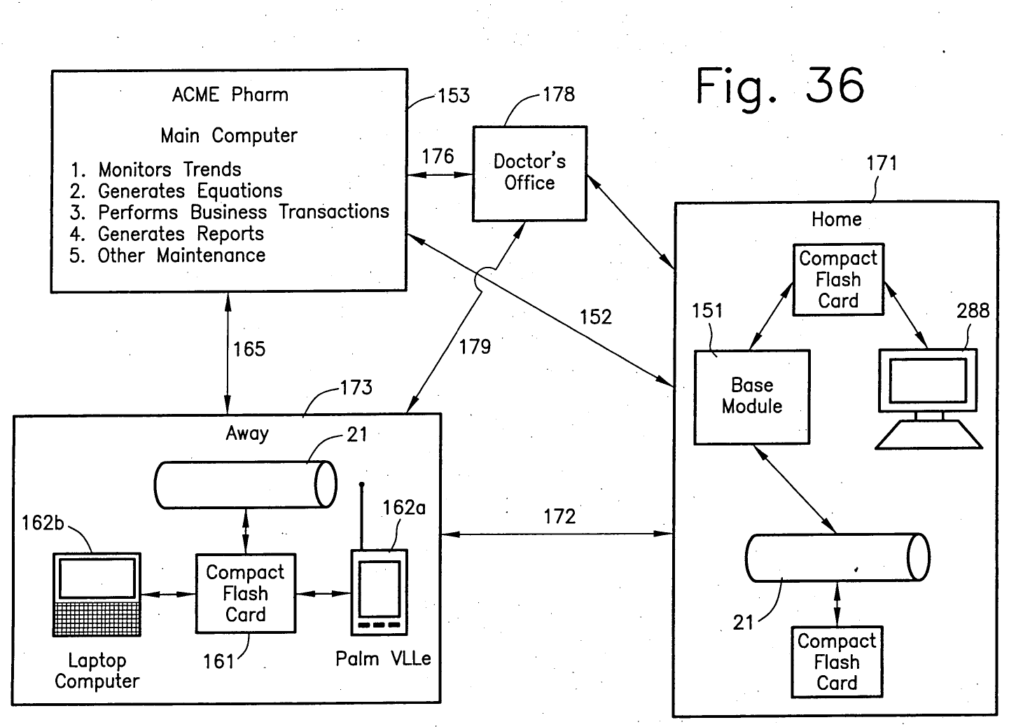

FIGS. 35A-B illustrate spectroscopic detector arrangements. As shown in FIG. 35A, the device includes a light emitting portion 214 and two detectors 215,216 that surround light emitting portion 214 and can be included in a hand-held wand device or in a table-top device, for example. Light emitting portion 214 has a light source that could be any light source, such as a quartz halogen lamp with integrated focusing optics or a fiber optic bundle, and light emitting portion 214 preferably has a rectangular prism SiO₂ light guide. At predetermined intervals, light emitting portion 214 emits light onto body part 10. Detectors 215,216 then detect the light reflected off body part 10. Detectors 215,216 are preferably formed of silicon and are preferably designed to detect only a specific range of wavelengths. For example, detector 215 could be set to detect light at wavelengths of only 400-700 nm, and detector 216 could be set to detect light at wavelengths of only 600-1100 nm. As such, the device shown in FIG. 13A would be able to detect light wavelengths of 400-1100 nm.

In one embodiment, detectors 215,216 can detect light at their specific wavelength ranges due to the presence above each filter 215,216 of an optical filter that restricts the transmission of light to detectors 215,216 at wavelengths in only the respective specified ranges.

In another embodiment, detectors 215,216 are array detectors and can detect light at their specific wavelength ranges due to the presence above each detector 215,216 of a linear variable filter 120, as shown in FIGS. 34A-B, that restricts the transmission of light to detectors 215,216 at wavelengths in only the specified, predetermined narrow band of wavelengths.

In a further preferred embodiment of a remote spectrometer, as shown in FIG. 35B, the device includes a light emitting portion 214 and three detectors 217,218,219 that surround light emitting portion 214. Light emitting portion 214 has a light source that could be any light source but is preferably a quartz halogen lamp with integrated focusing optics, and light emitting portion 214 preferably has a triangular prism SiO₂ light guide. Detectors 217,218,219 may each be disposed adjacent to a respective side of triangular light emitting portion 214, as depicted in FIG. 35B. At predetermined intervals light emitting portion 214 emits light onto body part 10. Detectors 217,218,219 then detect the light reflected off body part 10. The spectrometer of FIG. 35B is similar to the spectrometer of FIG. 35A, except that light emitting portion 214 is located among three detectors, rather than two detectors in FIG. 35A.

Detectors 217,218,219 are designed to detect only specific bands of wavelengths. For example, detectors 217,218,219 are preferably formed of silicon, with detector 217 detecting light at wavelengths of 400-700 nm, and detector 218 detecting light at wavelengths of 600-1100 nm. In addition, detector 219 is preferably formed of indium/gallium/arsenic (InGaAs) and detects light at wavelengths of 11-1900 nm. As such, the device can detect light wavelengths of 400-1900 nm. In one embodiment, detectors 217,218,219 can detect light at their specific wavelength ranges due to the presence above each detector 217,218,219 of an optical filter that restricts the transmission of light to detectors 217,218,219 at wavelengths in only the specified ranges. In another embodiment, detectors 217,218,219 are array detectors and can detect light at their specific wavelength ranges due to the presence above each detector 217,218,219 of a linear variable filter 120, as shown in FIGS. 34A-B, that restricts the transmission of light to detectors 217,218,219 at wavelengths in only the specified, predetermined narrow band of wavelengths.

Most preferably, the embodiments of FIGS. 35A-B may be used and positioned very much in the same way as filter 223 and detector 226 are used and positioned in the embodiments and versions discussed elsewhere herein, such as those shown in FIGS. 23A-G.

FIG. 36 depicts a system for predicting blood glucose values in a patient in which remote wireless spectrometer 21 interacts with central computer 153. “Wireless spectrometer” is intended to mean a spectrometer which transmits its data relating to spectral scans over a path which is at least partially wireless. Such a spectrometer is not physically connected to a device that interprets the spectral scan data. Wireless spectrometer 21, which can be made in accordance with any of the possible embodiments described above, may be considered to be situated at a location remote from central computer 153, for example at the home 171 of a patient. Spectrometer 21 is connected, either directly or wirelessly, to base module 151 that could also be situated at home 171 of the patient. Base module 151 may include a computer or other processing device. A home display device 288 may be provided. In certain embodiments of the present invention, one or both of base module 151 and display device 288 may form part of spectrometer 21.

In certain embodiments, remote communication link 152 is provided between base module 151 and central or main computer 153. This link 152 could be by wireless satellite cable, LAN, telephone link or any other suitable wireless connection, and could be directly from base module 151 to main computer 153. Main computer 153 receives and stores the spectral scan from the remote spectrometer. Main computer 153 may also monitor trends in successive spectral scans, perform analysis thereof, generate and regenerate a modeling equation for each sample as necessary, predict blood glucose values as described herein, generate reports, and perform business transactions and other tasks. Main computer 153 may transmit information to any of doctor's office 178 (or a hospital), home 171, and away location 173. Main computer 153 may be or include any suitable type of processing device. Main computer 153 may be located in any suitable place, such as a commercial or non-profit organization, a hospital, or a doctor's office.

In certain embodiments, remote communication link 174 bay be provided between base module 151 and doctor's office 178. Moreover, remote communication link 176 bay be provided between doctor's office 178 and main computer 153. In other embodiments of the present invention, remote communication links 174 and 176 may between a hospital and base module 151 and main computer 153.

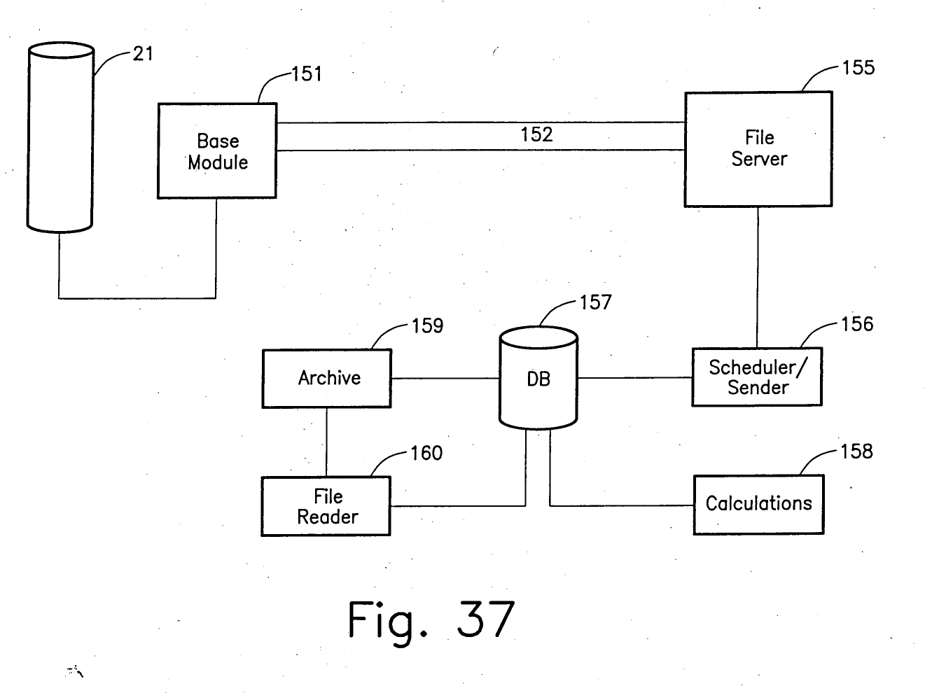

FIG. 37 shows in more particular detail the elements of a base connection to the main computer. Spectrometer 21 is connected, either directly or wirelessly, such as via a RS-232 Blue Tooth® Wireless link, to a base module 151, which may be a computer or other processing device located at home 171. The remote communication link 152 between base module 151 and main computer 153 can be additionally by existing dedicated telephone line, such as by dial-up modem, by wireless communication such as satellite cable, LAN, by internet, such as by cable or DSL, or even through a virtual private network (VPN) or any other suitable wireless connection. Main computer 153 preferably includes a file server 155 that is linked to a database 157 through a scheduler/sender 156. Database 157 is also linked to calculations 158, archive 159 and file reader 160 modules.

Referring again to FIG. 36, in certain circumstances, remote spectrometer 21 of the present invention can be transported and used at “away” location 173 removed from home 171. Spectrometer 21 could obtain the spectrographic data from a variety of different locations. Modeling equations and results can be stored on compact flash card 161, or other portable storage medium, that is attached to spectrometer 21. Spectrometer 21 can be connected, either directly or wirelessly, to portable base module 162, such as PALM®-type device 162a or laptop computer 162b, that typically comprises a processing unit and a display device. Portable base module 162 may be linked wirelessly over link 172 to base module 151 for downloading and compilation of data. Portable base module 162 could also be wirelessly linked over wireless link165 to main computer 153. Moreover, portable base module 162 could also be wirelessly linked over wireless link 179 to doctor's office 178. These links 172,165 and 179 could be by wireless satellite cable, LAN, telephone link or any other suitable wireless connection.

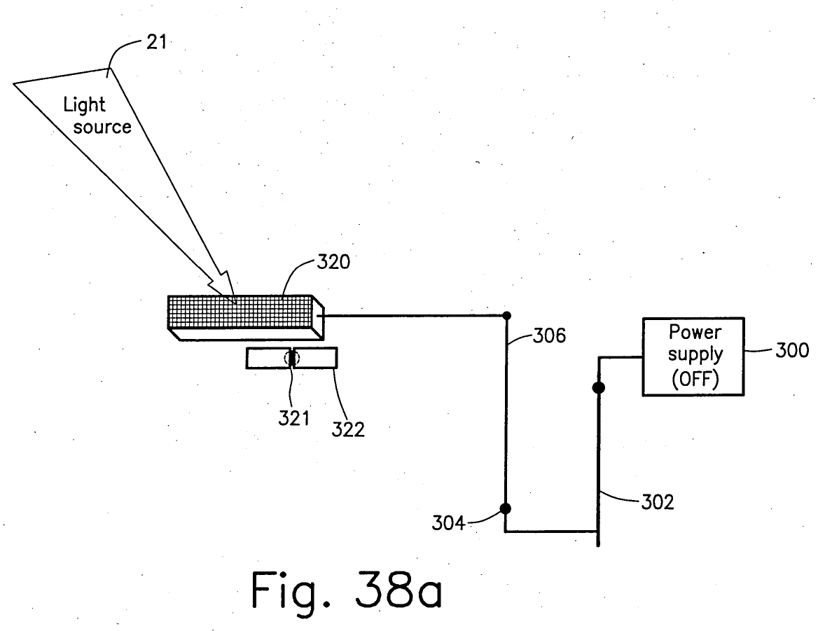

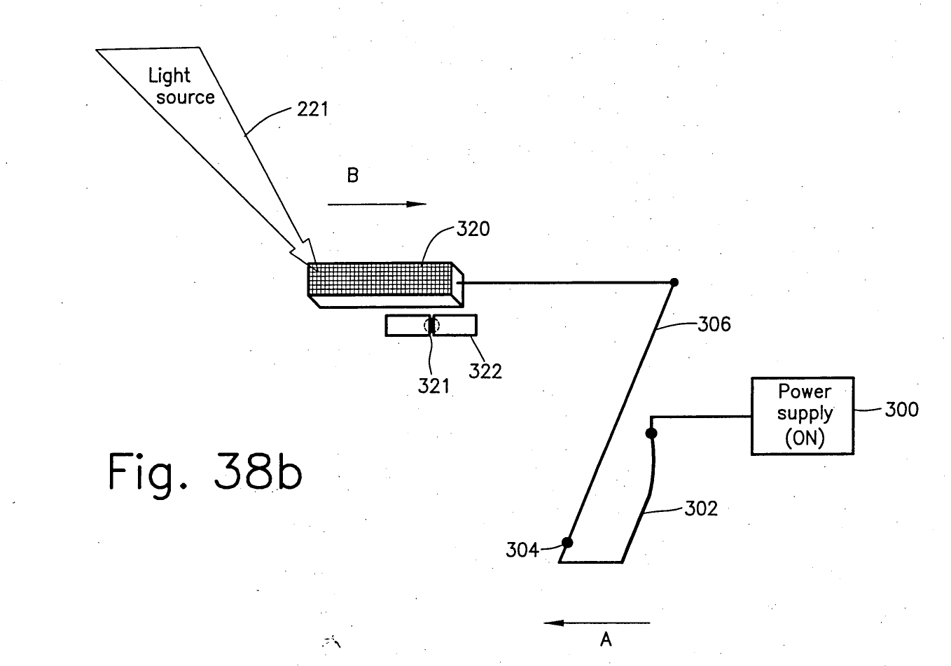

FIGS. 38A-B show a further embodiment of remote spectrometer 21. As illustrated in FIG. 38A, light source 221 produces a light beam that is passed through body part 10, through near infrared or infrared window/transparent element, through linear variable filter 323, through slit aperture 322 and onto single diode detector 321. As in the embodiment described above with reference to FIGS. 23A, the light from light source 221 may pass through near infrared or infrared window/transparent element 225. For example, spectrometer 21 can be located in a handheld device. Window/transparent element 225 may be quartz, sapphire, or glass, for example. After being transmitted through body part 10 (as shown, for example, in FIG. 23D), or reflected off of body part 10 (as shown, for example, in FIG. 23F), the light is passed through linear variable filter 320 in order in order to filter the light to a desired band of wavelengths. The light is then detected by the detector 321, either as transmittance or reflectance. In one embodiment, linear variable filter 320 can be arranged as a single range filter, and detector 321 is a single range detector, as shown in FIG. 38A.

The embodiment shown in FIGS. 38A-B is a scanning module because the device is equipped with piezoelectric bimorph (bender) 302 for moving linear variable filter 320 in various directions in order to allow the operator to obtain filtered scans of the body part 10 at a number of specific, predetermined narrow band of wavelengths in the light. Bimorph 302, powered by power supply 300, is connected to linear variable filter 320 via fulcrum 304 and lever 306, which amplify the displacement of the bimorph. FIG. 38A shows bimorph 302 with power supply 300 off. FIG. 38B shows bimorph 302 with power supply 300 on. With power supply 300 on, bimorph 302 bends as shown in FIG. 38B, forcing the lower portion of lever 306 to pivot about fulcrum 304 in the direction of arrow A. The pivoting of lever 306 causes linear variable filter 320 to move in the direction of arrow B, as indicated. To select each desired wavelength, power supply 300 may be controlled so as to provide predetermined power levels to bimorph 302 and thereby translate linear variable filter 320 to a desired position.

The embodiment of the invention shown in FIGS. 38A-B is “solid state” in the sense that no electric motor is used to move linear variable filter 320. Piezoelectric bimorph 302 may be capable of very precise and repeatable positioning to within fractions of a micron, allowing for advantageous wavelength reproducibility. Linear variable filter 320 may be, for example, 2-3 mm in length, thereby enabling a relatively small overall size of spectrometer 21. Spectrometer 21 may be used in a wavelength range from ultraviolet to the mid infrared (200 nm-10,000 nm) by selecting the appropriate combination of linear variable filter 320 and detector 321.

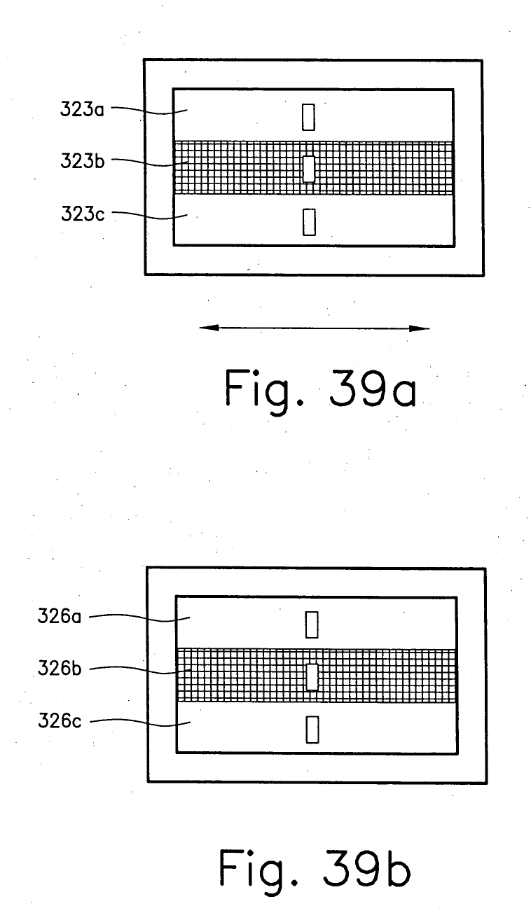

In another embodiment, linear variable filter 320 can be arranged as separate multi-range filters 323a,323b,323c, as shown in top view in FIG. 39A. In this embodiment, each of linear variable filters 323a,323b,323c restricts the transmission of light to wavelengths in only certain specified, predetermined narrow band of wavelengths. For example, linear variable filter 323a transmits light at wavelengths of 400-700 nm, linear variable filter 323b transmits light at wavelengths of 600-1100 nm, and linear variable filter 323c transmits light at wavelengths of 1100-1900 nm. The separate multi-range linear variable filters 323a, 323b, 323c may be moved by respective piezoelectric bimorphs in order to allow the operator to obtain filtered scans of product 11 at a number of specific, predetermined narrow band of wavelengths in the light. When separate multi-range filters 323a,323b,323c are used, the separate detectors may also be used to detect light at only those specific bands of wavelengths. For example, as shown in top view in FIG. 39B, detectors 326a,326b,326c are situated such that detector 326a detects light at wavelengths of 400-700 nm, detector 326b detects light at wavelengths of 600-1100 nm, and detector 326c detects light at wavelengths of 1100-1900 nm. As such, the device can detect light wavelengths of 400-1900 nm.

The operation of this device will be shown with regard to the multi-range filter and detector embodiment but applies equally to the single range filter and detector embodiment. The operator programs the processing device (not shown) as to the desired wavelengths or ranges of wavelengths to be scanned, and the piezoelectric biomorphs move linear variable filters 323a,323b,323c so as to allow only the desired wavelengths to pass. Thus, the light 21 is filtered to the desired band of wavelengths by linear variable filters 323a,323b,323c is focused onto array detectors 326a,326b,326c (or one for each of detectors 326a,326b,326c), which detect light at the specific wavelength ranges.

Alternatively, the operator may operate the device manually so as to allow scans to be taken at only the particular wavelengths specified at the time by the operator.

In other embodiments of the invention using bimorph 302, other types of detectors may be used in place of single diode detector 321. Preferably, a solid state detector is used.

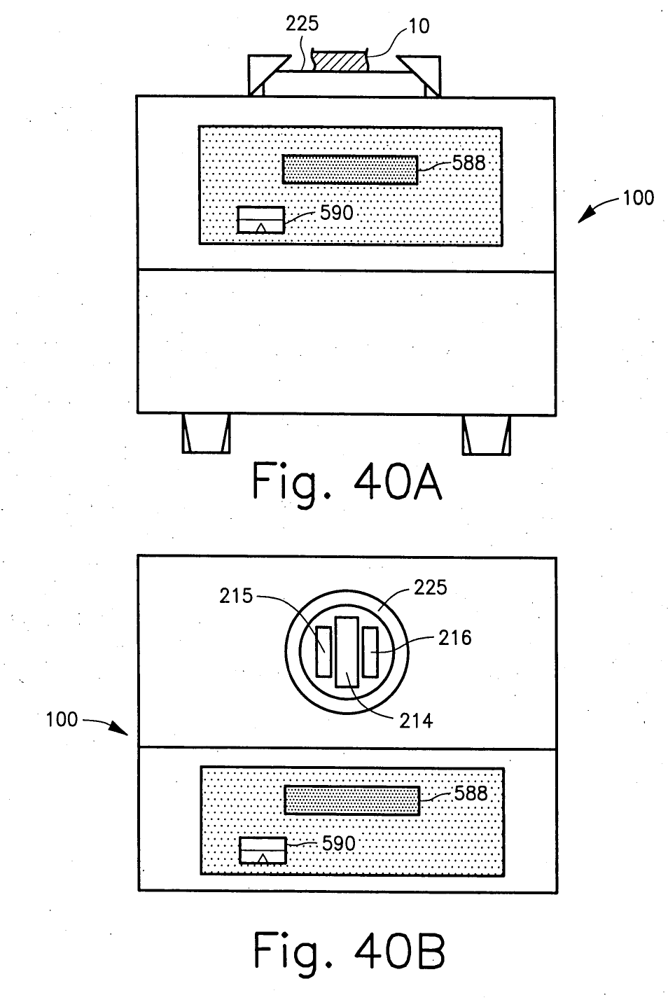

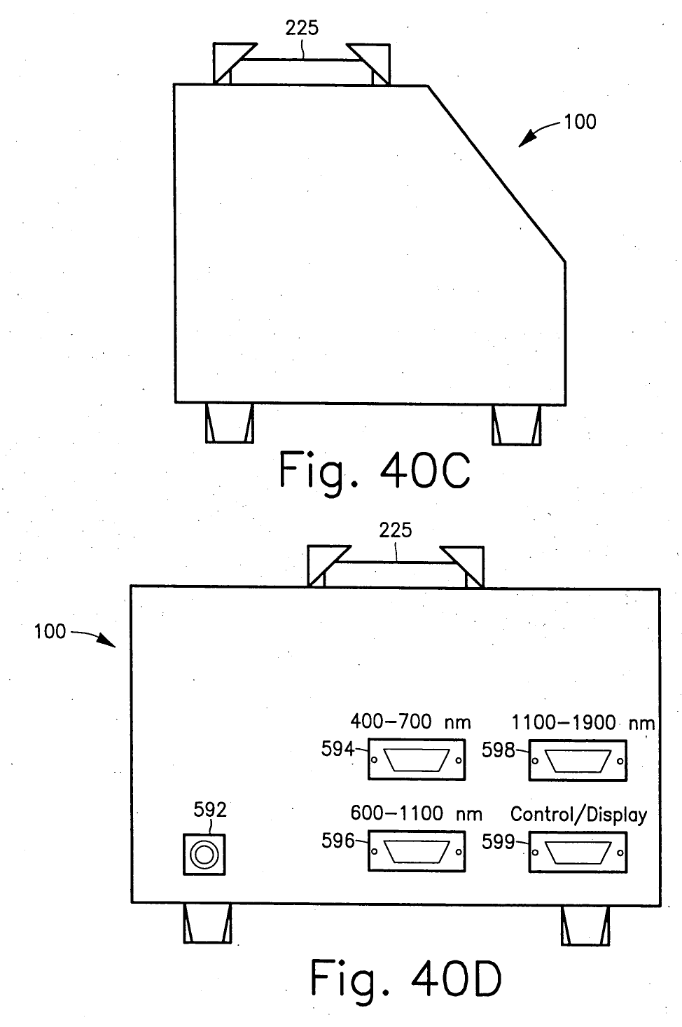

FIGS. 40A-D show various views of a table-top blood monitor device 100 according to an embodiment of the present invention. FIG. 40A show a front view of blood monitor device 100 including display 588 and scan initiator button 590. Transparent element 225 is provided for passing light to and from spectrometer 21 and body part 10, which is placed by the patient adjacent to transparent element 225 to perform a spectroscopic scan of the body part. Other than transparent element 225, spectrometer 21 is enclosed within table-top device 100 and not further shown in FIG. 40A.

FIG. 40B shows a top view of blood monitor device 100. Light emitting portion 214 emits light onto body part 10, while detectors 215,216 are provided for detecting light reflected off the body part, as discussed in more detail above with reference to FIG. 35A. In other embodiments of the present invention, a third detector may be provided, as described above with reference to FIG. 35B.

FIG. 40C shows a side view of blood monitor device 100.

FIG. 40D shows a back view of blood monitor device 100. Power input 592 as well as control display connector 599 are provided. Connectors 592 and 599 may each be any suitable respective connection type as would be understood by one of skill in the art. Connectors 594 and 596 are outputs from detectors 215,216 at 400-700 nm and 600-1100 nm, respectively. Connector 598 may be provided for the output of a third detector, when such a third detector is employed. Connectors 594,596,598 may be RS-232 connectors or any other suitable connector-type.

Many other variations of the present invention would be obvious to those skilled in the art and are contemplated to be within the scope of the appended claims. One skilled in the art will appreciate that the present invention can be practiced by other than the described embodiments, which are presented for purposes of illustration and not limitation.

1. A system for predicting blood glucose values in a patient, comprising:
a remote wireless non-invasive spectral device configured for generating a spectral scan of a body part of the patient;
a remote invasive device configured for generating a constituent value for the patient; and
a central processing device configured for predicting a blood glucose value for the patient based upon the spectral scan and the constituent value.

2. The system as recited in claim 1 wherein the central processing device is further configured for receiving at least one of the constituent value and information regarding the spectral scan over an at least partially wireless path.

3. The system as recited in claim 1 wherein the central processing device is further configured for receiving at least one of the constituent value and information regarding the spectral scan by a mode of data transmission.

4. The system as recited in claim 3 wherein the mode of data transmission is at least one of a cellular data link, a telephone modem, a direct satellite link, an Internet link, and an RS232 data connection.

5. The system as recited in claim 1 wherein the remote wireless non-invasive spectral device is configured for transmitting information regarding the spectral scan by a mode of data transmission.

6. The system as recited in claim 5 wherein the mode of data transmission is at least one of a cellular data link, a telephone modem, a direct satellite link, an Internet link, and an RS232 data connection.

7. The system as recited in claim 1 wherein the remote wireless non-invasive spectral device includes a wireless spectrometer.

8. The system as recited in claim 7 wherein the wireless spectrometer includes an infrared spectrometer.

9. The system as recited in claim 7 wherein the wireless spectrometer comprises a light source for irradiating the body part and at least one detector for detecting radiation reflected off or transmitted through the body part.

10. The system as recited in claim 9 wherein the at least one detector is on a side of the body part proximate to the light source for detecting light reflected off the body part.

11. The system as recited in claim 9 wherein the at least one detector is on a side of the body part remote from the light source for detecting light transmitted through the body part.

12. The system as recited in claim 9 wherein the light source emits radiation in multiple wavelengths, the system further comprising a filter for restricting passage of light through the filter in only a specific predetermined range of wavelengths.

13. The system as recited in claim 12 wherein the filter is situated between the light source and the body part, such that the filtering means allows passage of light in only a specific predetermined range of wavelengths to pass to the body part.

14. The system as recited in claim 12 wherein the filter is situated between the body part and the at least one detector, such that the filter allows passage of only a specific predetermined range of wavelengths reflected off or transmitted through the body part to pass to the at least one detector.

15. The system as recited in claim 12 wherein the filter is at least one linear variable filter.

16. The system as recited in claim 15 further comprising a solid state translation device operatively connected to the at least one linear variable filter and configured for moving the at least one linear variable filter.

17. The system as recited in claim 16 wherein the at least one detector comprises a plurality of individual detectors.

18. The system as recited in claim 16 wherein the solid state translation device is a piezoelectric bimorph.

19. The system as recited in claim 18 further comprising a lever device coupling the piezoelectric bimorph to the at least one linear variable filter and configured for amplifying a movement of the at least one linear variable filter relative to a movement of the piezoelectric bimorph.

20. The system as recited in claim 12 wherein the at least one detector is at least one array detector.

21. The system as recited in claim 12 wherein the at least one detector is at least one diode.

22. The system as recited in claim 12 wherein the filter is a bandpass filter.

23. The system as recited in claim 22 wherein the filter includes a plurality of bandpass filters.

24. The system as recited in claim 12 wherein the filter is a grating.

25. The system as recited in claim 24 wherein the grating is a diffraction grating.

26. The system as recited in claim 9 wherein the light source emits light in only a specific predetermined range of wavelengths, and wherein the at least one detector detects light reflected off or transmitted through the body part in the specific predetermined range of wavelengths.

27. The system as recited in claim 9 wherein the light source emits light in multiple wavelengths, and wherein each of the at least one detector detects light reflected off or transmitted through the body part in only a specific predetermined range of wavelengths.

28. The system as recited in claim 7 wherein the wireless spectrometer sends information regarding the spectroscopic data to the central processing device through infrared radiation or near infrared radiation.

29. The system as recited in claim 9 wherein the light source is capable of illuminating a plurality of positions in a region of the body part.

30. The system as recited in claim 29 wherein the light source includes a fiber optic bundle for illuminating the plurality of positions.

31. The system as recited in claim 30 wherein the light source includes a plurality of near-infrared light emitting diodes, each for illuminating a respective position of the plurality of positions.

32. The system as recited in claim 30 wherein the at least one detector is disposed in the region for detecting light reflected off or transmitted through the body part.

33. The system as recited in claim 32 wherein each of the at least one detector is configured for detecting a respective wavelength of light.

34. The system as recited in claim 30 further comprising:
a plurality of optical fibers spaced apart on the region for receiving radiation reflected off or transmitted through the body part and delivering the respective radiation to the at least one detector; and
a switching device coupled to each of the plurality of optical fibers and to the at least one detector, the switching device configured to connect one of the respective optical fiber at a time to the at least one detector.

35. The system as recited in claim 1 wherein the remote wireless non-invasive spectral device includes a transmitter configured for wirelessly transmitting information regarding the spectral scan.

36. The system as recited in claim 1 wherein the remote wireless non-invasive spectral device is handheld.

37. The system as recited in claim 1 wherein the remote wireless non-invasive spectral device includes a sensor, a monitor and a handheld processing device.

38. The system as recited in claim 1 further comprising a remote processing device configured for communicating with the central processing device.

39. The system as recited in claim 38 wherein the remote processing device is further configured for transmitting information regarding the spectral scan to the central processing device.

40. The system as recited in claim 38 wherein the remote wireless non-invasive spectral device is configured for transmitting information regarding the spectral scan to the remote processing device.

41. The system as recited in claim 38 wherein the remote processing device is further configured for transmitting information regarding the spectral scan to at least one of a doctor's office and a hospital.

42. The system as recited in claim 1 wherein the remote wireless non-invasive spectral device includes a wireless spectrometer, the wireless spectrometer including:
a light source;
a focusing optical device configured for focusing light from the light source onto the body part;
a linear variable filter device disposed so as to receive light transmitted through or reflected by the body part and pass light in at least one predetermined narrow wavelength band; and
an array detector device configured for receiving and detecting light from the linear variable filter device.

43. The system as recited in claim 1 wherein the remote wireless non-invasive spectral device includes a wireless spectrometer, the wireless spectrometer including:
a light source configured for emitting light onto the body part, the light source including a prism light guide; and
at least a first and a second detector disposed adjacent the light source, the at least first and second detector being configured for receiving light reflected from the body part.

44. The system as recited in claim 43 wherein the prism light guide is an SiO₂ rectangular prism light guide.

45. The system as recited in claim 43 wherein the prism light guide is an SiO₂ triangular prism light guide and wherein the at least first and second detector include a third detector, the first, second and third detector being disposed adjacent respective sides of the triangular prism light guide.

46. The system as recited in claim 1 wherein the remote wireless non-invasive spectral device includes a wireless spectrometer, the wireless spectrometer including:
a light source configured for emitting light onto the body part;
a linear variable filter device configured to pass light in at least one predetermined wavelength band and disposed so as to receive light transmitted through or reflected from the body part;
an array detector device configured for receiving the light passed by the linear variable filter device;
a detector imaging optic device configured for directing the light passed by the linear variable filter device onto the array detector device;
an enclosure configured for receiving the linear variable filter device, the detector imaging optic device and the array detector device; and
a transparent element disposed at a wall of the enclosure and configured for passing the light transmitted through or reflected by the body part to the linear variable filter.

47. The system as recited in claim 46 wherein the linear variable filter device includes a plurality of multi-range filters, each of the multirange filters passing a respective predetermined wavelength band.

48. The system as recited in claim 46 further comprising at least one drive device for moving the linear variable filter device so as to change the at least one predetermined wavelength band.

49. The system as recited in claim 1 wherein the remote wireless non-invasive spectral device is portable and further comprising a storage media device configured for storing at least one of information regarding the spectral scan and an equation for interpreting the spectral scan.

50. The system as recited in claim 1 wherein the remote wireless non-invasive spectral device is portable and configured for transmitting information regarding the spectral scan to the central processing device.

51. The system as recited in claim 1 further comprising a remote processing device configured for communicating with the central processing device and wherein the remote wireless non-invasive spectral device is portable and configured for transmitting information regarding the spectral scan to at least one of the central processing device and the remote processing device.

52. The system as recited in claim 1 wherein the remote invasive device is configured for taking a blood sample by a venipuncture, a fingerstick, and a heelstick so as to perform the generating.

53. The system as recited in claim 1 wherein the remote invasive device is configured for transferring the constituent value to the remote wireless non-invasive spectral device.

54. The system as recited in claim 1 wherein the remote invasive device is configured for transmitting at least one of the constituent value and information regarding the spectral scan by a mode of data transmission.

55. The system as recited in claim 1 wherein the central processing device is further configured for receiving the spectral scan from the remote wireless non-invasive spectral device and for storing a modeling equation for predicting blood glucose values in the patient, the central processing device being further configured for regenerating the modeling equation based upon a plurality of spectral scans of the patient from the remote wireless non-invasive spectral device and a corresponding plurality of constituent values for the patient from the remote invasive device, and for predicting a blood glucose value for the patient based upon a subsequent non-invasive spectral scan of the patient with the remote wireless non-invasive spectral device and the regenerated modeling equation.

56. The system as recited in claim 1 wherein the central processing device is further configured for transmitting information to at least one of the patient, a doctor's office and a hospital.

57. The system as recited in claim 1 wherein the central processing device includes a computer.

58. The system as recited in claim 1 further comprising a base module confiigured for receiving information regarding the spectral scan wirelessly from the remote wireless non-invasive spectral device and for communicating with the central processing device, and wherein the central processing device includes a file server and a data base device linked to the file server through a scheduler/sender device.

59. The system as recited in claim 1 wherein the remote wireless non-invasive spectral device is disposed at a home of the patient.

60. The system as recited in claim 1 wherein:
the remote wireless non-invasive spectral device is further configured for transmitting the spectral scan to the central processing device and for generating a plurality of second spectral scans of the body part;
the remote invasive device is further configured for generating a plurality of second constituent values for the patient respectively associated with the plurality of second spectral scans; and
the central processing device is further configured for:
dividing the plurality of second spectral scans and constituent values into a calibration subset and a validation subset;
transforming the second spectral scans in the calibration sub-set and the validation subset by applying a plurality of a first mathematical function to the calibration sub-set and the validation sub-set to obtain a plurality of transformed validation data sub-sets and a plurality of transformed calibration sub-sets;
resolving each transformed calibration data sub-set by at least one of a second mathematical function to generate a plurality of modeling equations;
selecting a best modeling equation of the plurality of modeling equations;
storing the best modeling equation in a central computer;
predicting the patient's blood glucose level using the best modeling equation; and
regenerating the best modeling equation if the spectral scan falls outside a range for the modeling equation.

61. The system as recited in claim 60 wherein the best modeling equation is selected as a function of calculating a figure of merit (FOM), the FOM being defined as:
FOM=\sqrt{(SEE²+2*SEP²)/3}
where:
SEE is the Standard Error of Estimate from the calculations on the calibration data;
SEP is the Standard Error of Estimate from the calculations on the validation data; and the modeling equation which provides the best correlation between the spectral data in the validation sub-set and the corresponding constituent values in the validation sub-set being identified as the modeling equation with the lowest FOM value.

62. The system as recited in claim 60 wherein the at least one second mathematical function includes one or more of a partial least squares, a principal component regression, a neural network, and a multiple linear regression analysis.

63. The system as recited in claim 60 wherein the first set of mathematical functions include performing a normalization of the spectral scan, performing a first derivative on the spectral scan, performing a second derivative on the spectral scan, performing a multiplicative scatter correction on the spectral scan, performing smoothing transform on the spectral scan, a Savitsky-Golay first derivative, a Savitsky-Golay second derivative, a mean-centering, a Kubelka-Munk transform, and a conversion from reflectance/transmittance to absorbence.

64. The system as recited in claim 60 wherein the first set of mathematical functions are applied singularly and two-at-a-time.

65. The system as recited in claim 60 wherein the remote wireless non-invasive spectral device is further configured for transmitting the spectral scan to the central processing device over an at least partially wireless transmission path.

66. A system for predicting blood glucose values in a patient, comprising:
a remote wireless non-invasive spectral device configured for generating a spectral scan of a body part the patient;
a remote invasive device configured for generating a constituent value for the patient; and
a central processing device configured for receiving the spectral scan from the remote wireless non-invasive spectral device and for storing a modeling equation for predicting blood glucose values in the patient, the central processing device predicting a blood glucose value for the patient based upon the spectral scan and the modeling equation, the central processing device regenerating the modeling equation based upon a plurality of spectral scans of the patient from the remote wireless non-invasive spectral device and a corresponding plurality of constituent values for the patient from the remote invasive device, and predicting a blood glucose value for the patient based upon a subsequent non-invasive spectral scan of the patient with the remote wireless non-invasive spectral device and the regenerated modeling equation.

67. A method for predicting blood glucose values in a patient, comprising:
generating a spectral scan of a body part the patient using a remote wireless non-invasive spectral device;
generating a constituent value for the patient using a remote invasive device; and
predicting a blood glucose value for the patient using a central processing device based upon the spectral scan and the constituent value.

68. The method as recited in claim 67 further comprising:
receiving the spectral scan from the remote wireless non-invasive spectral device;
storing a modeling equation for predicting blood glucose values in the patient using the central processing device;
regenerating the modeling equation using the central processing device based upon a plurality of spectral scans of the patient from the remote wireless non-invasive spectral device and a corresponding plurality of constituent values for the patient from the remote invasive device; and
predicting a blood glucose value for the patient based upon a subsequent non-invasive spectral scan of the patient with the remote wireless non-invasive spectral device and the regenerated modeling equation.

69. The method as recited in claim 67 wherein the remote spectral device includes an infrared spectrometer.

70. The method as recited in claim 67 wherein the infrared spectrometer includes a grating spectrometer, a diode array spectrometer, a filter-type spectrometer, an Acousto Optical Tunable Filter spectrometer, a scanning spectrometer, an ATR spectrometer, and a nondispersive spectrometer.

71. The method as recited in claim 67 wherein the remote wireless non-invasive spectral device communicates with the central processing device by a mode of data transmission.

72. The method as recited in claim 67 wherein the spectral device controls administering an amount of a drug to the patient.

73. The method as recited in claim 67 wherein the central processing device includes a workstation capable of holding a plurality of spectral scans and modeling equations for a plurality of patients.

74. An automated method for predicting blood glucose values using a noninvasive spectroscopic technique, comprising the steps of:
(a) taking a plurality of measurements of a patient's blood glucose levels using a noninvasive spectral device and an invasive glucose monitoring method;
(b) associating a constituent value measured by the invasive glucose monitoring method with the blood glucose level measured by the spectral device;
(c) dividing the plurality of spectral scans and constituent values into a calibration subset and a validation subset;
(d) transforming the spectral scans in the calibration sub-set and the validation subset by applying a plurality of a first mathematical function to the calibration sub-set and the validation sub-set to obtain a plurality of transformed validation data sub-sets and a plurality of transformed calibration sub-sets;
(e) resolving each transformed calibration data sub-set in step (d) by at least one of a second mathematical function to generate a plurality of modeling equations; and
(f) selecting a best modeling equation of the plurality of modeling equations;
(g) storing the best modeling equation in a central computer;
(h) acquiring a spectral scan from the patient using a remote wireless noninvasive spectral device;
(i) transmitting the spectral scan from step (h) to the central computer of step (g);
(j) predicting the patient's blood glucose level using the best modeling equation; and
(k) regenerating the best modeling equation if the spectral scan falls outside a range for the modeling equation.

75. The method as recited in claim 74 wherein the best modeling equation is selected as a function of calculating a figure of merit (FOM), the FOM being defined as:
FOM=\sqrt{(SEE²+2*SEP²)/3}
where:
SEE is the Standard Error of Estimate from the calculations on the calibration data;
SEP is the Standard Error of Estimate from the calculations on the validation data; and the modeling equation which provides the best correlation between the spectral data in the validation sub-set and the corresponding constituent values in the validation sub-set being identified as the modeling equation with the lowest FOM value.

76. The method as recited in claim 74 wherein the at least one second mathematical function includes one or more of a partial least squares, a principal component regression, a neural network, and a multiple linear regression analysis.

77. The method as recited in claim 74 wherein the first set of mathematical functions include performing a normalization of the spectral scan, performing a first derivative on the spectral scan, performing a second derivative on the spectral scan, performing a multiplicative scatter correction on the spectral scan, performing smoothing transform on the spectral scan, a Savitsky-Golay first derivative, a Savitsky-Golay second derivative, a mean-centering, a Kubelka-Munk transform, and a conversion from reflectance/transmittance to absorbence.

78. The method as recited in claim 74 wherein the first set of mathematical functions are applied singularly and two-at-a-time.

79. The method as recited in claim 74 wherein the transmitting of step (i) takes place over an at least partially wireless transmission path.
